(19) United States
(12) Patent Application Publication
(10) Pub. No.: US 2019/0008779 A1
(43) Pub. Date: Jan. 10, 2019

(54) GEMCABENE COMPOSITIONS AND METHODS OF USE THEREOF

(71) Applicant: GEMPHIRE THERAPEUTICS INC., Livonia, MI (US)

(72) Inventors: Daniela Carmen ONICIU, Gainesville, FL (US); Charles Larry Bisgaier, Ann Arbor, MI (US); Janice Cacace, St. Petersburg, FL (US); Matthew Benjamin Greene, Tampa, FL (US)

(21) Appl. No.: 15/977,226

(22) Filed: May 11, 2018

Related U.S. Application Data
(60) Provisional application No. 62/505,085, filed on May 11, 2017.

(51) Int. Cl. A61K 9/28; A61K 31/40; A61K 31/194; A61K 9/48

(52) U.S. Cl. CPC A61K 9/2846; A61K 9/4808; A61K 31/194; A61K 31/40

(57) ABSTRACT

The present invention provides pharmaceutical compositions comprising a statin and an outer coating, and optionally gemcabene, and methods of use thereof.

### CROSS-REFERENCE TO RELATED APPLICATION

This application claims the benefit of U.S. Provisional Application No. 62/505,085, filed May 11, 2017, the disclosure of which is incorporated by reference herein in its entirety.

### FIELD OF THE INVENTION

This invention relates to pharmaceutical compositions comprising a statin or a pharmaceutically acceptable salt thereof and an outer coating. The invention also relates to pharmaceutical compositions comprising a statin or a pharmaceutically acceptable salt thereof and gemcabene or a pharmaceutically acceptable salt thereof. These pharmaceutical compositions are useful for treating disorders of lipoprotein metabolism, disorders of glucose metabolism, cardiovascular disorders, diseases of the liver, diseases of the kidney, diseases of the lung, disease of the muscle and inflammation.

### BACKGROUND

Separate administration of gemcabene calcium and a statin has been shown to significantly reduce plasma levels of LDL cholesterol (LDL-C) below that of a statin alone. In addition, gemcabene calcium has been shown to further reduce LDL-C levels in patients on a stable dose of statin that are not able to reach the target LDL-C goal. Furthermore, in patients having type IIb hyperlipidemia, certain doses of gemcabene calcium and a statin show surprising ability to lower triglycerides when compared with either gemcabene alone or statin alone. In addition, administration of gemcabene calcium and a statin has been shown to reduce c-reactive protein to an extent greater than the statin treatment alone. Further, some doses of gemcabene calcium and a statin lower fibrinogen in hypercholesterolemic human subjects with elevated fibrinogen levels.

Although gemcabene calcium administration has been shown not to significantly affect the pharmacokinetics of simvastatin and atorvastatin in vivo, prior attempts at the formulation of combination tablets by means of common tableting techniques reduced stability of such formulations. For instance, common tableting of atorvastatin calcium with poly (vinyl pyrrolidone) (PVP) and gemcabene calcium generates a drug product that possesses a reduced shelf life due to the rapid formation of product-related degradation impurities. Similarly, in other tablets that are formulated with atorvastatin calcium and acidic excipients or acidic drugs, atorvastatin has shown poor shelf-life. Therefore, creation of long term stable formulations that comprise gemcabene or a pharmaceutically acceptable salt thereof and a statin has been challenging.

Atorvastatin has been observed to undergo an acid-mediated conversion to an undesirable lactone in the stomach. Moreover, two other atorvastatin metabolites, 2-hydroxy-atrovastatin and 4-hydroxy-atorvastatin, are active towards HMG-CoA reductase, and undergo lactonization in the gastrointestinal tract along with atorvastatin (FIGS. 1A and 1B).

### SUMMARY OF THE INVENTION

In some embodiments, the invention provides a tablet comprising (a) a core, wherein the core comprising a statin or a pharmaceutically acceptable salt thereof and (b) an outer coating, wherein the outer coating comprising a copolymer comprising methyl acrylate, methyl methacrylate and methacrylic acid repeat units in a ratio of (about 7):(about 3):(about 1); methacrylic acid and ethyl acrylate repeat units in a ratio of (about 1):(about 1); or a combination thereof, wherein the core has an outer surface and wherein the outer coating is disposed over the entire outer surface. A tablet comprising the core comprising statin and the outer coating comprising the copolymer as disclosed herein is a “tablet of the invention”.

In some embodiments, the tablet of the invention comprises (a) a core, wherein the core comprising a statin or a pharmaceutically acceptable salt thereof and (b) an outer coating, wherein the outer coating comprising a first copolymer or a second copolymer, wherein the first copolymer comprising methyl acrylate, methyl methacrylate and methacrylic acid repeat units in a ratio of (about 7):(about 3):(about 1), and the second copolymer comprising methacrylic acid and ethyl acrylate repeat units in a ratio of (about 1):(about 1).

In some embodiments, the invention provides a capsule containing the tablet of the invention comprising (a) a core, wherein the core comprising a statin or a pharmaceutically acceptable salt thereof and (b) an outer coating, wherein the outer coating comprising a first copolymer or a second copolymer, wherein the first copolymer comprising methyl acrylate, methyl methacrylate and methacrylic acid repeat units in a ratio of (about 7):(about 3):(about 1), and the second copolymer comprising methacrylic acid and ethyl acrylate repeat units in a ratio of (about 1):(about 1).

In some embodiments, the invention provides a kit comprising (i) the tablet of the invention comprising (a) a core, wherein the core comprising a statin or a pharmaceutically acceptable salt thereof and (b) an outer coating, wherein the outer coating comprising a first copolymer or a second copolymer, wherein the first copolymer comprising methyl acrylate, methyl methacrylate and methacrylic acid repeat units in a ratio of (about 7):(about 3):(about 1), and the second copolymer comprising methacrylic acid and ethyl acrylate repeat units in a ratio of (about 1):(about 1), (ii) a pharmaceutical composition comprising gemcabene or a pharmaceutically acceptable salt thereof, and (iii) instructions for use of the tablet or the pharmaceutical composition.

In some embodiments, the invention provides an oral dosage form comprising: (a) a first composition comprising (1) an effective amount of a statin or a pharmaceutically acceptable salt thereof and (2) a pharmaceutically acceptable carrier; and (b) a second composition comprising (1) an effective amount of gemcabene or a pharmaceutically acceptable salt thereof and (2) a pharmaceutically acceptable carrier, wherein the first composition is surrounded by the second composition.

In some embodiments, the invention provides methods for treating or preventing a liver disease or an abnormal liver condition, comprising administering to a subject in need thereof an effective amount of the oral dosage form of the invention, including the tablet and the capsule. In some embodiments, the invention provides methods for treating or preventing a disorder of lipoprotein metabolism, comprising administering to a subject in need thereof an effective amount of the oral dosage form of the invention, including the tablet and the capsule.

In some embodiments, the invention provides methods for reducing a subject's total cholesterol level, low density lipoprotein cholesterol concentration, low density lipoprotein concentration, very low density lipoprotein cholesterol concentration, very low density lipoprotein concentration, non-HDL cholesterol concentration, non-HDL concentration, apolipoprotein B level, triglyceride concentration, apolipoprotein C-III level, C-reactive protein level, fibrinogen level, or lipoprotein(a) level in the subject's blood plasma or serum, comprising administering to a subject in need thereof an effective amount of the oral dosage form of the invention, including the tablet and the capsule. In some embodiments, the invention provides a method for elevating a subject's high density lipoprotein cholesterol concentration, high density lipoprotein concentration, or apolipoprotein A-I level in the subject's blood plasma or serum, comprising administering to a subject in need thereof an effective amount of the oral dosage form of the invention, including the tablet and the capsule.

In some embodiments, the invention provides methods for reducing ballooning or inflammation in a liver of a subject, comprising administering to a subject in need thereof an effective amount of the oral dosage form of the invention, including the tablet and the capsule. In some embodiments, the invention provides methods for treating or preventing post-prandial lipemia, comprising administering to a subject in need thereof an effective amount of the oral dosage form of the invention, including the tablet and the capsule.

In some embodiments, the invention provides methods for treating or preventing a disorder of glucose metabolism, comprising administering to a subject in need thereof an effective amount of the oral dosage form of the invention, including the tablet and the capsule. In some embodiments, the invention provides methods for treating or preventing a cardiovascular disorder or a related vascular disorder, comprising administering to a subject in need thereof an effective amount of the oral dosage form of the invention, including the tablet and the capsule. In some embodiments, the invention provides methods for treating or preventing a C-reactive protein-related disorder, comprising administering to a subject in need thereof an effective amount of the oral dosage form of the invention, including the tablet and the capsule.

In some embodiments, the invention provides methods for treating or preventing Alzheimer's disease, Parkinson's disease or pancreatitis, comprising administering to a subject in need thereof an effective amount of the oral dosage form of the invention, including the tablet and the capsule. In some embodiments, the invention provides methods for treating or preventing a pulmonary disorder, comprising administering to a subject in need thereof an effective amount of the oral dosage form of the invention, including the tablet and the capsule.

In some embodiments, the invention provides methods for treating or preventing musculoskeletal discomfort, comprising administering to a subject in need thereof an effective amount of the oral dosage form of the invention, including the tablet and the capsule.

### BRIEF DESCRIPTION OF THE DRAWINGS

FIGS. 1A and 1B are schemes showing lactone-dihydroxy acid interconversion. In FIG. 1A, “p-Hydroxy Atorvastatin” can also be referred to as “4-hydroxyatorvastatin”; “p-Hydroxy Atorvastatin Lactone” can also be referred to as “4-hydroxyatorvastatin lactone”; “o-Hydroxy Atorvastatin” can also be referred to as “2-hydroxyatorvastatin”; and “o-Hydroxy Atorvastatin Lactone” can also be referred to as “2-hydroxyatorvastatin lactone”.

FIG. 1C is a line graph showing a dissolution profile of atorvastatin from atorvastatin calcium capsules (20 mg) of Example 3.

FIG. 2 is a line graph showing a dissolution profile of gemcabene from gemcabene calcium capsules (150 mg) of Example 4.

FIG. 4 is a line graph showing a dissolution profile of atorvastatin from enteric coated atorvastatin calcium tablets, 20 mg (formulation 1 of Example 5).

FIG. 5 is a line graph showing a dissolution profile of atorvastatin from enteric coated atorvastatin calcium tablets, 20 mg (formulation 2 of Example 6).

FIG. 6 is a line graph showing a dissolution profile of atorvastatin from enteric coated atorvastatin calcium tablets, 20 mg (formulation 3 of Example 7).

FIG. 7 is a line graph showing a dissolution profile of atorvastatin from enteric coated atorvastatin calcium tablets, 20 mg (formulation 4 of Example 8).

FIG. 8 is a line graph showing a dissolution profile of atorvastatin from enteric coated atorvastatin calcium tablets, 20 mg (formulation 5 of Example 9).

FIG. 9A is a graph showing the pharmacokinetic profiles of total atorvastatin (non-lactone and lactone), atorvastatin non-lactone, and atorvastatin lactone metabolites for phase 1—atorvastatin calcium capsule of Example 3, 1×20 mg (Example canine A).

FIG. 9B is a graph showing the pharmacokinetic profiles of total atorvastatin (non-lactone and lactone), atorvastatin non-lactone, and atorvastatin lactone metabolites for phase 3 (PR 1)—atorvastatin calcium tablet, 1×20 mg formulation 1 (Example 5) in gemcabene calcium capsule 1×150 mg (Example 4) (Example canine A).

FIG. 9C is a graph showing the pharmacokinetic profiles of total atorvastatin (non-lactone and lactone), atorvastatin non-lactone, and atorvastatin lactone metabolites for phase 4 (PR 2)—atorvastatin calcium tablet, 1×20 mg formulation 2 (Example 6) in gemcabene calcium capsule 1×150 mg (Example 4) (Example canine A).

FIG. 10A is a graph showing the pharmacokinetic profiles of total atorvastatin (non-lactone and lactone), atorvastatin non-lactone, and atorvastatin lactone metabolites for phase 1—atorvastatin calcium capsule of Example 3, 1×20 mg (Example canine C).

FIG. 10B is a graph showing the pharmacokinetic profiles of total atorvastatin (non-lactone and lactone), atorvastatin non-lactone, and atorvastatin lactone metabolites for phase 3 (PR 1)—atorvastatin calcium tablet, 1×20 mg formulation 1 (Example 5) in gemcabene calcium capsule 1×150 mg (Example 4) (Example canine C).

FIG. 10C is a graph showing the pharmacokinetic profiles of total atorvastatin (non-lactone plus lactone), atorvastatin non-lactone, and atorvastatin lactone metabolites for phase 4 (PR 2)—atorvastatin calcium tablet, 1×20 mg formulation 2 and gemcabene calcium capsule 1×150 mg (Example 4) (Example canine C).

FIG. 11A is a graph showing the pharmacokinetic profiles of total atorvastatin (non-lactone plus lactone), atorvastatin non-lactone, and atorvastatin lactone metabolites for phase 5—LIPITOR® (atorvastatin calcium) tablet, 1×40 mg (Example canine A).

FIG. 11B is a graph showing the pharmacokinetic profiles of total atorvastatin (non-lactone and lactone), atorvastatin non-lactone, and atorvastatin lactone metabolites for phase 6 (PR 3)—atorvastatin calcium tablets, 2×20 mg formulation 3 (Example 7) (Example canine A).

FIG. 11C is a graph showing the pharmacokinetic profiles of total atorvastatin (non-lactone plus lactone), atorvastatin non-lactone, and atorvastatin lactone metabolites for phase 7 (PR 4)—atorvastatin calcium tablets, 2×20 mg formulation 4 (Example 8) (Example canine A).

FIG. 12A is a graph showing the pharmacokinetic profiles of total atorvastatin (non-lactone and lactone), atorvastatin non-lactone, and atorvastatin lactone metabolites for phase 5—LIPITOR® (atorvastatin calcium) tablet, 1×40 mg (Example canine B).

FIG. 12B is a graph showing the pharmacokinetic profiles of total atorvastatin (non-lactone and lactone), atorvastatin non-lactone, and atorvastatin lactone metabolites for phase 6 (PR 3)—atorvastatin calcium tablets, 2×20 mg formulation 3 (Example 7) (Example canine B).

FIG. 12C is a graph showing the pharmacokinetic profiles of total atorvastatin (non-lactone and lactone), atorvastatin non-lactone, and atorvastatin lactone metabolites for phase 7 (PR 4)—atorvastatin calcium tablets, 2×20 mg formulation 4 (Example 8) (Example canine B).

FIG. 13A is a graph showing the pharmacokinetic profiles of total atorvastatin (non-lactone and lactone), atorvastatin non-lactone, and atorvastatin lactone metabolites for phase 5—LIPITOR® (atorvastatin calcium) tablet, 1×40 mg (Example canine C).

FIG. 13B is a graph showing the pharmacokinetic profiles of total atorvastatin (non-lactone and lactone), atorvastatin non-lactone, and atorvastatin lactone metabolites for phase 6 (PR 3)—atorvastatin calcium tablets, 2×20 mg formulation 3 (Example 7) (Example canine C).

FIG. 14A is a graph showing the pharmacokinetic profiles of total atorvastatin (non-lactone and lactone), atorvastatin non-lactone, and atorvastatin lactone metabolites for phase 5—LIPITOR® (atorvastatin calcium) tablet, 1×40 mg (Example canine D).

FIG. 14B is a graph showing the pharmacokinetic profiles of total atorvastatin (non-lactone and lactone), atorvastatin non-lactone, and atorvastatin lactone metabolites for phase 6 (PR 3)—atorvastatin calcium tablets, 2×20 mg formulation 3 (Example 7) (Example canine D).

FIG. 14C is a graph showing the pharmacokinetic profiles of total atorvastatin (non-lactone and lactone), atorvastatin non-lactone, and atorvastatin lactone metabolites for phase 7 (PR 4)—atorvastatin calcium tablets, 2×20 mg formulation 4 (Example 8) (Example canine D).

FIG. 15 is a graph showing the pharmacokinetic profile of gemcabene (composite data from all animals), as follows: a) phase 2—gemcabene calcium of Example 4, b) phase 3—atorvastatin calcium formulation 1 of Example 5 in gemcabene calcium of Example 4, and c) phase 4—atorvastatin calcium formulation 2 of Example 6 in gemcabene calcium of Example 4.

FIG. 16 is a graph showing the pharmacokinetic profiles in plasma of total atorvastatin (non-lactone and lactone), atorvastatin non-lactone, and atorvastatin lactone metabolites for phase 5—LIPITOR® (atorvastatin calcium) tablet, 1×40 mg. The graph depicts values that are the average of the values obtained from the four dogs in the experimental group.

FIG. 17 is a graph showing the pharmacokinetic profiles in plasma of total atorvastatin (non-lactone and lactone), atorvastatin non-lactone, and atorvastatin lactone metabolites for phase 6 (PR 3)—atorvastatin calcium tablets, 2×20 mg formulation 3 (Example 7). The graph depicts values that are the average of the values obtained from the four dogs in the experimental group.

FIG. 18 is a graph showing the pharmacokinetic profiles in plasma of total atorvastatin (non-lactone and lactone), atorvastatin non-lactone, and atorvastatin lactone metabolites for phase 7 (PR 4)—atorvastatin calcium tablets, 2×20 mg formulation 4 (Example 8). The graph depicts values that are the average of the values obtained from the three dogs in the experimental group.

FIG. 19 is a graph showing the pharmacokinetic profiles in plasma of total atorvastatin (non-lactone and lactone), atorvastatin non-lactone, and atorvastatin lactone metabolites for phase 8 (PR 5)—atorvastatin calcium tablets, 2×20 mg formulation 5 (Example 9). The graph depicts values that are the average of the values obtained from the four dogs in the experimental group.

### DETAILED DESCRIPTION OF THE INVENTION

The present invention provides compositions of the invention. In some embodiments, a composition of the invention comprises a statin or a pharmaceutically acceptable salt thereof and gemcabene or a pharmaceutically acceptable salt thereof. In some embodiments, the composition is an oral dosage form. In some embodiments, the oral dosage form is a capsule. In some embodiments, the capsule is a tablet-in-capsule.

The compositions of the invention are useful for treating or preventing various diseases, disorders and conditions, including liver disease or an abnormal liver condition, a disorder of lipoprotein or glucose metabolism, a cardiovascular or related vascular disorder, a disease caused by increased levels of fibrosis, or a disease associated with increased inflammation.

In some embodiments, the compositions of the invention reduce or eliminate drug-drug and excipient-drug interactions during storage of compositions of statins and gemcabene as a combined fixed dose form. In some embodiments, the compositions of the invention produce a different release profile of the statin compared to the release profile of the gemcabene in order to improve the pharmacokinetics of the composition and its constituents. In some embodiments, the compositions of the invention comprise a statin or a pharmaceutically acceptable salt thereof and gemcabene or a pharmaceutically acceptable salt thereof and at least one pharmaceutically acceptable excipient. An excipient may reduce or eliminate stability issues during storage of gemcabene and a statin in one composition (for example, a combined dose form). In some embodiments, the pharmaceutical compositions of the invention are fixed dose compositions with modified pharmacokinetics for reducing adverse effects that would require the discontinuation of administration of the composition to a subject.

In some embodiments, the invention provides a pharmaceutical composition that is a tablet-in-capsule, wherein the pharmaceutical composition comprises from about 0.1 mg to about 80 mg of a statin or a pharmaceutically acceptable salt thereof; from about 50 mg to about 900 mg of gemcabene or a pharmaceutically acceptable salt thereof, and one or more pharmaceutically acceptable excipients. In some embodiments, the composition comprises: a tablet comprising from about 0.1 mg to about 80 mg of a statin or a pharmaceutically acceptable salt thereof, and a capsule comprising from about 50 mg to about 900 mg of gemcabene or a pharmaceutically acceptable salt thereof, wherein the capsule encompasses both the tablet comprising a statin and the gemcabene. In some embodiments, the statin is a HMG-CoA reductase inhibitor. In some embodiments, the statin is atorvastatin, simvastatin, pravastatin, rosuvastatin, fluvastatin, lovastatin, dalvastatin, dihydrocompactin, cerivastatin or pitavastatin. In some embodiments, the statin is atorvastatin. In some embodiments, the statin is atorvastatin calcium. In some embodiments, the gemcabene is gemcabene calcium. In some embodiments, the gemcabene is formulated as an immediate release formulation. In some embodiments, the atorvastatin is formulated as a delayed release formulation. In some embodiments, the atorvastatin formulation allows up to 5% release of atorvastatin in the stomach.

In some embodiments, the composition comprises an atorvastatin calcium tablet core comprising: a) about 13 to about 14 wt % atorvastatin calcium; b) about 39 to about 41 wt % lactose monohydrate; c) about 22 to about 23 wt % calcium carbonate; d) about 18 to about 20 wt % microcrystalline cellulose; e) about 1.5 to about 2.5 wt % polyvinylpyrrolidone; 0 about 0.2 to about 0.3 wt % polysorbate 80; g) about 2 to about 3 wt % croscaramellose sodium; and h) about 0.3 to about 0.5 wt % magnesium stearate.

In some embodiments, the invention provides a modified release atorvastatin and gemcabene fixed dose formulation in the form of any of their salts with a lag phase before atorvastatin delivery suitable for oral once a day administration for treating lipid disorders without causing or reducing drug-induced hepatotoxicity and musculoskeletal disorders. In some embodiments, the invention provides a modified release atorvastatin and gemcabene fixed dose formulation in the form of any of their salts with a lag phase before atorvastatin delivery suitable for oral once a day administration for treating lipid disorders wherein the drug-induced hepatotoxicity and musculoskeletal disorders is reduced or eliminated.

In some embodiments, the invention provides a modified release atorvastatin and gemcabene fixed dose combination formulation or any of its salts with a lag phase before atorvastatin delivery suitable for oral once a day administration for treating lipid disorders where the atorvastatin component exhibits a release pattern characterized by two phases, a lag phase and an extended release phase; wherein the lag phase is characterized in that less than 10% of the absorbable atorvastatin dose administered is absorbed between about 0.5 and about 1 hour following ingestion; wherein the extended release phase being characterized in that more than about 20% but less than 78% of the absorbable atorvastatin administered being absorbed between about 1.5 and 4 hours following ingestion; and wherein less than 90% of the absorbable atorvastatin administered being absorbed by 9 hours following ingestion.

In some embodiments, the invention provides a gemcabene microparticle having a coating ratio of about 2.5% to about 15%, wherein the amount of gemcabene is about 80% to about 98%, the amount of ethylcellulose is about 1% to about 10%, the amount of castor oil is about 0.01% to about 1.5%, the amount of povidone is about 0.05% to about 1%, the amount of tartaric acid is about 0% to about 1%, and the amount of magnesium stearate is about 0% to about 2%.

In some embodiments, the invention provides an atorvastatin microparticle having a coating ratio of about 10% to about 30%, wherein the amount of atorvastatin is about 60% to about 95%, the amount of methacrylic acid copolymer type C (L100-55) is about 0% to about 15%, the amount of methacrylic acid copolymer type B (S100) is about 0% to about 15%, and the amount of cottonseed oil is about 0% to about 15%.

In some embodiments, the invention provides a pharmaceutical formulation comprising a capsule filled with gemcabene microparticles and an atorvastatin calcium microtablet, said microtablet comprising (i) a core comprising about 10 to about 80% atorvastatin calcium, about 15 to about 60% lactose monohydrate, about 10 to about 25% microcrystalline cellulose, about 0 to about 10% polyvinylpyrrolidone, about 0 to about 10% croscaramellose sodium, about 0 to about 10% magnesium stearate; (ii) a subcoat barrier of about 1 to about 5% weight gain relative to the core weight comprising a suitable excipient such as Opadry or mixtures of suitable excipients; (iii) an enteric coating composition applied at about 2 to about 15% weight relative to the core weight, comprising methacrylic acid, methyl acrylate, methyl methacrylate copolymer of about 0% to about 10%, methacrylic acid copolymer type C of about 10% to about 0%, and triethyl citrate of about 0% to about 2%.

In some embodiments, the invention provides a pharmaceutical composition, comprising gemcabene calcium from about 50 mg to about 900 mg, and atorvastatin calcium from about 5 mg to about 80 mg, and a pharmaceutically acceptable carrier, wherein said gemcabene is released about 50% at about 4 to about 6 hours with a Tₘₐₓ at about 1 to about 2 hours, and wherein said atorvastatin is released from the composition with a lag time of about 1.5 to about 4 hours.

In some embodiments, the invention provides a pharmaceutical composition comprising atorvastatin microparticles having a pH-dependent release profile, and gemcabene microparticles having a pH-independent release profile, wherein the atorvastatin microparticles have a reduced capacity to provoke musculoskeletal reactions in a subject, wherein the gemcabene is present in an amount effective to reduce triglycerides and LDL-cholesterol with at least 10% in addition to the effect of atorvastatin alone, and wherein there is a lag time between release of atorvastatin or gemcabene following administration of the composition.

In some embodiments, the invention provides a use of the pharmaceutical composition the invention for manufacturing of a medicament for treating or preventing a disease or disorder selected from: a) disorders of lipoprotein metabolism, wherein the disorder is dyslipidemia, dyslipoproteinemia, lipoprotein overproduction or deficiency, elevation of total cholesterol, elevation of low density lipoprotein concentration, elevation of triglyceride concentration, lipid elimination in bile, metabolic disorder, phospholipid elimination in bile, oxysterol elimination in bile, abnormal bile production, or peroxisome proliferator activated receptor-associated disorder; (b) disorders of glucose metabolism, wherein the disorder is insulin resistance, impaired glucose tolerance, impaired fasting glucose levels in blood, diabetes mellitus, lipodystrophy, central obesity, peripheral lipoatrophy, diabetic nephropathy, diabetic retinopathy, renal disease, or septicemia; (c) cardiovascular disorders and related vascular disorders, wherein the disorder is atherosclerosis, hypertension, coronary artery disease, myocardial infarction, arrhythmia, atrial fibrillation, heart valve disease, heart failure, cardiomyopathy, myopathy, pericarditis, impotence, or thrombotic disorder; d) diseases of the liver including NAFLD, NASH, alcoholic steatohepatitis, cirrhosis, inflammation fibrosis, primary biliary cirrhosis; (e) modulating inflammation markers and/or C-reactive protein and related disorders, wherein the disorder is inflammation, ischemic necrosis, or thrombotic disorder; and (0 aging, Alzheimer's Disease, Parkinson's disease, pulmonary disorders, and pancreatitis.

### Definitions

As used herein, the terms “pharmaceutically active agent”, “active pharmaceutical ingredient”, and “active pharmaceutical agent” are used interchangeably to refer to a biologically active compound. Examples of pharmaceutically active agents include, without limitation, gemcabene or a pharmaceutically acceptable salt thereof, statin or a pharmaceutically acceptable salt thereof, or any combination thereof.

As used herein, the terms polyvinylpyrrolidone (PVP), polypovidone, and povidone are used interchangeably and have the same meaning.

As used herein, the term “statin” refers to a class of pharmaceutically active agents or drugs that inhibit the enzyme HMG-CoA reductase and are generally known to lower LDL cholesterol in patients. Non-limiting examples of statins include atorvastatin, simvastatin, pravastatin, rosuvastatin, fluvastatin, lovastatin, dalvastatin, dihydrocompactin, cerivastatin and pitavastatin, and pharmaceutically acceptable salts thereof.

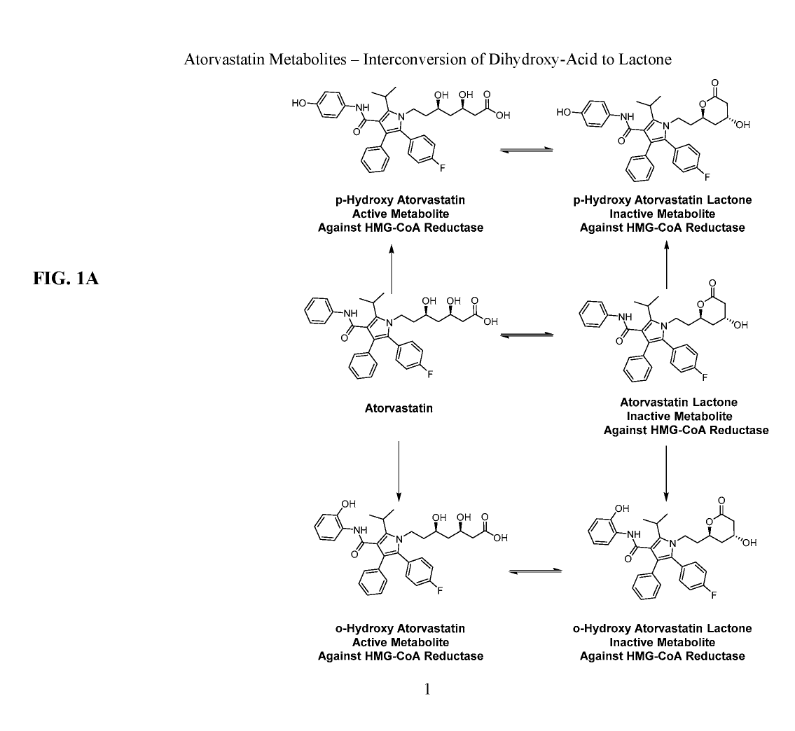

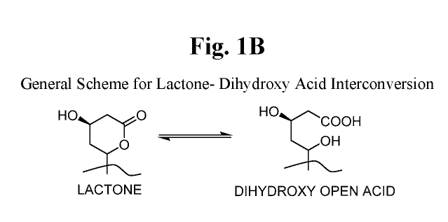

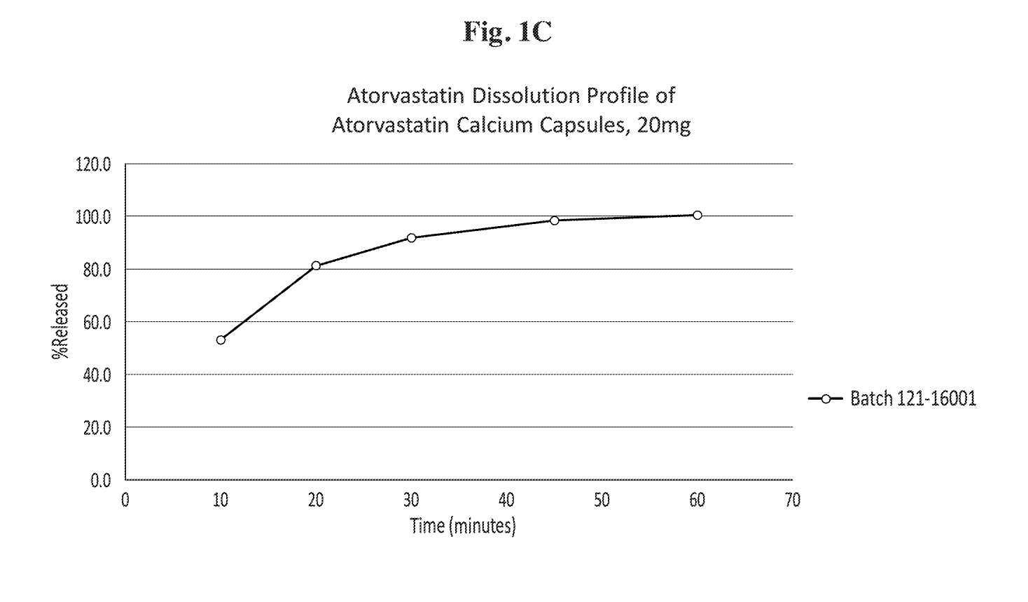

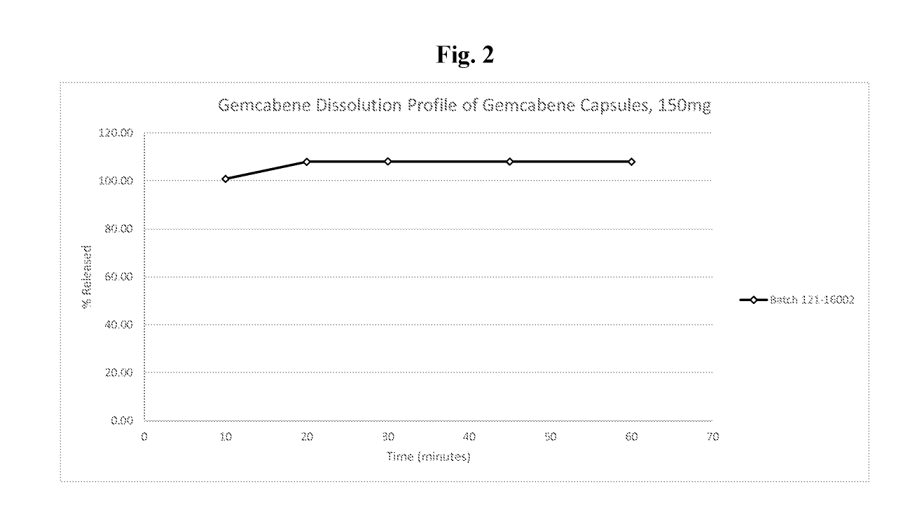

As used herein, the term “atorvastatin” refers to the compound labeled “atorvastatin” in FIG. 1A. As used herein, the terms “atorvastatin lactones” and “total lactone” refers to the total detected concentration of atorvastatin lactone+2-hydroxyatorvastatin lactone+4-hydroxyatorvastatin lactone. As used herein, the terms “atorvastatin non-lactones” and “total non-lactone”refers to the total detected concentration of atorvastatin+2-hydroxyatorvastatin+4-hydroxy atorvastatin.

Atorvastatin is administered orally as the calcium salt of the active hydroxyl acid form. It is well absorbed but has a low oral bioavailability, which is approximately 14% due to substantial first-pass metabolism (Lennernas, 2003). The pharmacologically active atorvastatin (acid) is biotransformed to its corresponding lactone form via a coenzyme A-dependent or an acyl glucuronide intermediate pathway (Kearney et al., 1993; Prueksaritanont et al., 2002; Lennernas, 2003). Both atorvastatin and atorvastatin lactone are further metabolized to form hydroxylated metabolites, primarily via cytochrome P450 (CYP) 3A4 enzyme-mediated metabolic pathway (Jacobsen et al., 2000). The lactone forms of atorvastatin and its metabolites can also be hydrolyzed back into their corresponding acid forms nonenzymatically or by esterases and paraoxonases (Kearney et al., 1993; Billecke et al., 2000; Prueksaritanont et al., 2002). Atorvastatin has also shown to be a substrate of the efflux transporter P-glycoprotein (P-gp) and organic anion transporting polypeptide (OATP1B1) (Konig et al., 2000; Wu et al., 2000). The polymorphism of OATP1B1 can affect the pharmacokinetic profiles and exposure of atorvastatin (Pasanen et al., 2007). The major hydroxylated metabolites, 2-hydroxy-atorvastatin acid and 4-hydroxy-atorvastatin acid (FIG. 1A), are pharmacologically equipotent to parent atorvastatin and significantly contribute to the inhibitory activity on HMG-CoA reductase during treatment (Lennernas, 2003). The pharmacologically inactive lactone forms, atorvastatin lactone, 2-hydroxy-atorvastatin lactone and 4-hydroxy-atorvastatin lactone, have been suggested to be associated with the adverse events of muscle toxicity and cause statin-induced myopathy (SIM) and rhabdomyolysis (Hermann et al., 2006, Skottheim et al., 2011).

Atorvastatin is subject to extensive metabolism to produce significant amount of active or toxic metabolites, whose exposure contributes significantly to the safety and efficacy, specifically statin-induced myopathy. Therefore, reliable prediction of drug disposition for atorvastatin and its metabolites is critical in its clinical dose regimen design, especially when atorvastatin is concomitantly dosed with other drugs. Such metabolism is characteristic for the whole class of statins. FIG. 1B depicts the lactone-dihydroxy acid interconversion.

As used herein, the term “statin lactones” refers to metabolites of a statin or a pharmaceutically acceptable salt thereof having a lactone moiety (e.g., see FIG. 1B).

As used herein, the term “gemcabene” refers to the compound 6,6′-oxybis(2,2-dimethylhexanoic acid) having the structure

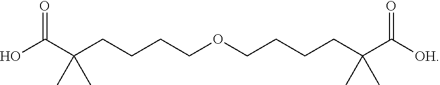 [SMILES: CC(C)(CCCCOCCCCC(C)(C)C(=O)O)C(=O)O]

A composition of the invention may comprise gemcabene or a pharmaceutically acceptable salt thereof. In some embodiments, a pharmaceutically acceptable salt of gemcabene is gemcabene calcium salt. In some embodiments, a composition of the invention comprises gemcabene calcium salt hydrate. Various gemcabene calcium salt hydrates have been previously disclosed in U.S. Pat. No. 6,861,555, which is hereby incorporated by reference in its entirety. In some embodiments, a composition of the invention comprises a crystalline polymorph of gemcabene. In some embodiments, a composition of the invention comprises gemcabene calcium salt hydrate Crystal Form 1. In other embodiments, a composition of the invention comprises gemcabene calcium salt hydrate Crystal Form 2. In other embodiments, a composition of the invention comprises gemcabene calcium salt hydrate Crystal Form C1. In other embodiments, a composition of the invention comprises gemcabene calcium salt hydrate Crystal Form C2. In other embodiments, a composition of the invention comprises gemcabene calcium salt hydrate Crystal Form C3. Gemcabene calcium salt hydrate Crystal Forms C1, C2, and C3 demonstrates variable extent of crystallinity. In some embodiments, a composition of the invention comprises amorphous gemcabene calcium salt. In some embodiments, a composition of the invention comprises amorphous gemcabene calcium salt hydrate.

All weight percentages (i.e., “% by weight” and “wt %” and w/w) referenced herein, unless otherwise indicated, are relative to the total weight of the mixture or composition, as the case can be.

As used herein, the term “tablet” can be any reasonably sized tablet suitable for oral ingestion. In some embodiments a “tablet” is a “minitablet” or “microtablet”. As used herein, a “microtablet” refers to a tablet having a diameter ranging from about 1 mm to about 5 mm.

As used herein, the term “excipient” refers to an inactive ingredient in a pharmaceutical composition. Examples of excipients include fillers or diluents, wetting agents (e.g., surfactants), binders, glidants, lubricants, disintegrants, or the like.

As used herein, a “disintegrant” is an excipient that in some embodiments hydrates a pharmaceutical composition and aids in tablet dispersion. Examples of disintegrants include sodium croscarmellose and/or sodium starch glycolate.

As used herein, a “diluent” or “filler” is an excipient that in some embodiments adds bulkiness to a pharmaceutical composition. Examples of fillers include lactose, sorbitol, celluloses, calcium phosphates, starches, sugars (e.g., mannitol, sucrose, or the like) or any combination thereof.

As used herein, a “wetting agent” or a “surfactant” is an excipient that in some embodiments imparts pharmaceutical compositions with enhanced solubility and/or wetability. Examples of wetting agents include sodium lauryl sulfate (SLS), sodium stearyl fumarate (SSF), polyoxyethylene 20 sorbitan mono-oleate (e.g., TWEEN®), or any combination thereof.

As used herein, a “binder” is an excipient that in some embodiments imparts a pharmaceutical composition with enhanced cohesion or tensile strength (e.g., hardness). Examples of binders include dibasic calcium phosphate, sucrose, corn (maize) starch, microcrystalline cellulose, modified cellulose (e.g., hydroxymethyl cellulose (HMC) or hydroxypropyl cellulose (HPC)), and polyvinylpyrrolidone (PVP).

As used herein, a “glidant” is an excipient that in some embodiments imparts a pharmaceutical compositions with enhanced flow properties. Examples of glidants include colloidal silica and/or talc.

As used herein, a “colorant” is an excipient that in some embodiments imparts a pharmaceutical composition with a desired color. Examples of colorants include commercially available pigments such as FD&C Blue #1 Aluminum Lake, FD&C Blue #2, other FD&C Blue colors, titanium dioxide, iron oxide, and/or combinations thereof. Other colorants include commercially available pigments such as FD&C Green #3.

As used herein, a “lubricant” is an excipient that in some embodiments is added to pharmaceutical compositions that are pressed into tablets. The lubricant aids in compaction of granules into tablets and ejection of a tablet of a pharmaceutical composition from a die press. Examples of lubricants include magnesium stearate, stearic acid (stearin), hydrogenated oil, sodium stearyl fumarate, or any combination thereof.

As used herein, the term “immediate release” or “IR” refers to an oral dosage form formulated to release the pharmaceutically active agent immediately upon ingestion.

As used herein, the term “extended release” or “ER” or “sustained release” or “SR” refers to an oral dosage form formulated to make the pharmaceutically active agent available over an extended period of time.

As used herein, the term “modified release” or “MR” refers to an oral dosage form formulated to modulate the pharmaceutically active agent's release from that of an IR dosage form. This can include ER/SR formulations, delayed release formulations such as enteric coated drug products, and targeted delivery drug products such as those intending to release the pharmaceutically active agent at a specific physiological location. Modified release dosage forms include delayed-, extended-, prolonged-, extended-, pulsatile- or pulsed-, controlled-, accelerated- and fast-, targeted-, programmed-release, and/or gastric retention dosage forms. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active agent(s) can also be modified by varying the particle sizes and polymorphism of the active agent(s).

The term “about” when immediately preceding a numerical value means±up to 10% of the numerical value. For example, “about 40” means±up to 10% of 40 (i.e., from 36 to 44), ±up to 10%, ±up to 9%, ±up to 8%, ±up to 7%, ±up to 6%, ±up to 5%, ±up to 4%, ±up to 3%, ±up to 2%, ±up to 1%, ±up to less than 1%, or any other value or range of values therein.

“Treating” when used in connection with a disease or disorder encompasses one or more of: (1) causing a regression of the disease or disorder; (2) stabilizing the disease or disorder; (3) slowing the progression of the disease or disorder; and (4) slowing the onset of a disease or a disorder or one or more of the symptoms or parameters of the disease or a disorder.

As used herein, the term “patient in need” or “subject in need” refers to a patient or subject at risk of, or suffering from, a disease, disorder or condition that is amenable to treatment or amelioration with a pharmaceutical composition provided herein. “Subject” and “patient” are used interchangeably herein.

In certain embodiments, a subject may be a human, a non-human primate, a pig, a horse, a cow, a dog, a cat, a mouse or a rat. In some embodiments, the subject is in a fed state when a composition disclosed herein is administered. In some embodiments, the subject is in a fasting state when a composition disclosed herein is administered.

### Pharmaceutical Compositions

In some embodiments, the present invention provides a pharmaceutical composition comprising a statin or a pharmaceutically acceptable salt thereof and gemcabene or a pharmaceutically acceptable salt thereof. In some embodiments, the present invention provides a pharmaceutical composition comprising a statin or a pharmaceutically acceptable salt thereof, gemcabene or a pharmaceutically acceptable salt thereof and one or more pharmaceutically acceptable excipients. In some embodiments, the pharmaceutical composition comprises atorvastatin or a pharmaceutically acceptable salt thereof and gemcabene or a pharmaceutically acceptable salt thereof.

In some embodiments, the present invention provides a pharmaceutical composition comprising a statin or a pharmaceutically acceptable salt thereof and gemcabene or a pharmaceutically acceptable salt thereof, wherein the statin or pharmaceutically acceptable salt thereof is present in the composition in an amount ranging from about 0.1 wt % to about 61.5 wt % of the total weight of the composition; and wherein the gemcabene or pharmaceutically acceptable salt thereof is present in the composition in an amount ranging from about 38.5 wt % to about 99.9 wt % of the total weight of the composition.

In some embodiments, the present invention provides a pharmaceutical composition comprising a statin or a pharmaceutically acceptable salt thereof and gemcabene or a pharmaceutically acceptable salt thereof, wherein the statin or pharmaceutically acceptable salt thereof is present in the composition in an amount ranging from about 2 wt % to about 35 wt % of the total weight of the composition; and wherein the gemcabene or pharmaceutically acceptable salt thereof is present in the composition in an amount ranging from about 65 wt % to about 98 wt % of the total weight of the composition.

In some embodiments, the present invention provides a pharmaceutical composition comprising a statin or a pharmaceutically acceptable salt thereof and gemcabene or a pharmaceutically acceptable salt thereof, wherein the statin or pharmaceutically acceptable salt thereof is present in the composition in an amount ranging from about 2 wt % to about 21 wt % of the total weight of the composition; and wherein the gemcabene or pharmaceutically acceptable salt thereof is present in the composition in an amount ranging from about 79 wt % to about 98 wt % of the total weight of the composition.

In some embodiments, the statin is a HMG-CoA reductase inhibitor. In some embodiments, the statin is a hepatoselective statin. In some embodiments, the statin is atorvastatin, simvastatin, pravastatin, mevastatin, fluvastatin, dalvastatin, dihydrocompactin, cerivastatin, lovastatin, pitavastatin or rosuvastatin; or a pharmaceutically acceptable salt of any of the previously listed statins. In some embodiments, the statin is atorvastatin, simvastatin, or a pharmaceutically acceptable salt of atorvastatin or simvastatin. In other instances, the statin is a calcium salt of atorvastatin.

In some embodiments, a pharmaceutical composition of the invention comprises a statin or a pharmaceutically acceptable salt thereof, gemcabene or a pharmaceutically acceptable salt thereof and one or more additional pharmaceutically active agents.

In some embodiments, the additional pharmaceutically active agent is a lipid-reducing agent. In certain embodiments, a lipid-reducing agent is ezetimibe, nicotinic acid, gemfibrozil, bempedoic acid, niacin, a bile-acid binding resin, a fabric acid derivative, a cholesterol absorption inhibitor or a PCSK9 (proprotein convertase subtilisin/kexin type 9) inhibitor.

In further embodiments, the pharmaceutical composition of the invention comprises from about 10 mg to about 300 mg bempedoic acid (for example from about 20 mg to about 280 mg, from about 30 mg to about 260 mg, from about 40 mg to about 240 mg, from about 60 mg to about 220 mg, from about 80 mg to about 200 mg, from about 100 mg to about 200 mg, from about 120 mg to about 180 mg, from about 50 mg to about 100 mg, from about 50 mg to about 150 mg, from about 100 mg to about 150 mg, or from about 150 mg to about 300 mg). In some embodiments, the pharmaceutical composition of the invention comprises about 10 mg, about 20 mg, about 30 mg, about 40 mg, about 50 mg, about 60 mg, about 70 mg, about 80 mg, about 90 mg, about 100 mg, about 120 mg, about 140 mg, about 150 mg, about 160 mg, about 180 mg, about 200 mg, about 220 mg, about 240 mg, about 250 mg, about 260 mg, about 280 mg, or about 300 mg bempedoic acid.

In some embodiments, the additional pharmaceutically active agent is an anti-inflammatory agent, an anti-hypertensive agent, an anti-diabetic agent, an anti-obesity agent, an anti-fibrotic agent or an anti-coagulation agent.

In some embodiments, the present invention provides a pharmaceutical composition that is a single administration unit (e.g., capsule, tablet, tablet-in-capsule) comprising a statin or a pharmaceutically acceptable salt thereof and gemcabene or a pharmaceutically acceptable salt thereof. In some embodiments, the statin is atorvastatin. In some embodiments, the pharmaceutical composition comprises statin and/or gemcabene microparticles (e.g., microcapsules, microbeads, microtablets). In some embodiments, the statin and the gemcabene have different pharmacokinetics from each other. For example, the gemcabene may be released immediately and the release may be sustained over an extended period. The statin may be released during an extended and sustained period (for example, released over about 8 hours) not immediately after administration, but with a lag time in statin release from administration of about 2 to about 4 hours. The lag is identified as the transit time from the administration of the pharmaceutical composition to a targeted site of release in the digestive system where it is absorbed from the intestines and measurable as the time of appearance of the agent or its metabolite in the plasma.

In some embodiments of the invention, the lag time for statin delivery is based on the design of a pharmaceutical composition able to protect the statin from fast release in the stomach, duodenum and jejunum, with release occurring in the distal part of the small intestine, i.e., delaying release to a region of the small intestine where the pH levels are elevated and do not favor formation of statin lactones. In some embodiments of the invention, the pharmaceutical composition utilizes pH-controlled release of pharmaceutically active agents. In some embodiments, the pharmaceutical composition of the invention does not allow release of a statin until after the composition passes the stomach.

The human intestinal system is characterized by variation of the pH in its different segments. Fallingborg, et al., Aliment Pharmacol Ther. 1989, 3, 605-13, describes a reference clinical study of the variations in pH and the residence times in various parts of the gastrointestinal tract by means of recording parameters for a pH-sensitive, radiotransmitting capsule. An exemplary overview providing an estimate of the pH in different sections of and residence time in the digestive tract is provided in Table 1 based on data from the clinical study in Fallingborg et al.

TABLE 1
pH Variation in the Gastrointestinal Tract and Residence
Time of a Capsule

| | Stomach | Duodenum | Jejunum | Ileum | Colon |
|---|---|---|---|---|---|
| Fasting pH | 1.4-2.1 | 4.6 | 4.4-6.6 | 6.8-8.6 | 5-8 |
| Time, hr | 1.2-2.1 | 2.4-6.8 | 6-7 | ~6 | ~17 hr |
| Fed pH | 3-5 | 4.5-5.5 | 5.2-6.2 | 6.8-8.0 | 5-8 |
| Time, hr | 0.1 | 1 | 2 | No data (>6) | ~17 hr |

In some embodiments, a pharmaceutical composition of the invention comprises a film coating agent. Film coating agents may ensure site specific delivery in the intestine. Most enteric coatings work by presenting a surface that is stable at the highly acidic pH found in the stomach but that breaks down rapidly at a relatively more basic pH. For example, some film coating agents will not dissolve in the acidic juices of the stomach (pH about 0.3) but will dissolve in the alkaline (pH about 7 to about 9) environment present in the distal small intestine. Various methacrylic acid copolymers have targeted drug release areas due to their dissolution at specific pH.

In some embodiments, a pharmaceutical composition of the invention comprises Methacrylic Acid Copolymer type C (such as, Eudragit® L100-55, also known as poly(methacrylic acid-co-methyl methacrylate) or poly(methyl methacrylate-co-methacrylic acid)). The targeted drug release area of Eudragit® L100-55 is the upper bowel and its dissolution pH is about 6 or above pH of about 5.5. In some embodiments, a pharmaceutical composition of the invention comprises Methacrylic Acid Copolymer type B (such as, Eudragit® S100, also known as poly(methacrylic acid-co-methyl methacrylate) or poly(methyl methacrylate-co-methacrylic acid)). The targeted drug release area of Eudragit® S100 is the colon and its dissolution pH is about 7 or above pH of about 7.

In some embodiments, a pharmaceutical composition of the invention comprises pH-dependent enteric polymers. In some embodiments, enteric polymers include, but are not limited to, methacrylic acid copolymers, methacrylic and methacrylate acid copolymers, cellulose acetate phthalate (CAP), cellulose acetate butyrate, hydroxypropylmethylcellulose phthalate (HPMCP), algenic acid salts such as sodium or potassium alginate, or shellac. In some embodiments, enteric polymers include poly(methacylic acid-co-methyl methacrylate) anionic copolymers based on methacrylic acid and methyl methacrylate. Poly(meth)acrylates (methacrylic acid copolymer), available under the trademark Eudragit® (Evonik Industries AG, Germany), are provided as powder or aqueous dispersions. In some embodiments, the methacrylic acid copolymer comprises Eudragit® L30D55; Eudragit® L100-55; Eudragit® L100; Eudragit® L12.5; Eudragit® S100; Eudragit® S12.5; Eudragit® FS30D; or combinations thereof.

In some embodiments, a pharmaceutical composition of the invention comprises an enteric coating comprising one or more enteric polymers. In some embodiments, a pharmaceutical composition of the invention comprises an enteric coating comprising a combination of at least two enteric polymers. In some embodiments, a pharmaceutical composition of the invention comprises an enteric coating comprising a combination of at least two enteric polymers as an outer coating. In some embodiments, a pharmaceutical composition of the invention comprises an enteric coating comprising a combination of Eudragit® L30D55 and Eudragit® FS30D. In one embodiment, a pharmaceutical composition of the invention comprises an enteric coating comprising a combination of Eudragit® L30D55 and Eudragit® FS30D in a ratio of about 1:5% w/w to about 5:1% w/w. In one embodiment, a pharmaceutical composition of the invention comprises an enteric coating comprising a combination of Eudragit® L30D55 and Eudragit® FS30D in a ratio of about 1:3 w/w to about 3:1 w/w. In one embodiment, a pharmaceutical composition of the invention comprises an enteric coating comprising a combination of Eudragit® L30D55 and Eudragit® FS30D in a ratio of about 1:2.5% w/w to about 2.5:1% w/w. In one embodiment, a pharmaceutical composition of the invention comprises an enteric coating comprising a combination of Eudragit® L30D55 and Eudragit® FS30D in a ratio of about 1:2% w/w to about 2:1% w/w. In one embodiment, a pharmaceutical composition of the invention comprises an enteric coating comprising a combination of Eudragit® L30D55 and Eudragit® FS30D in a ratio of about 1:2 w/w to about 1.5:1 w/w. In one embodiment, a pharmaceutical composition of the invention comprises an enteric coating comprising a combination of Eudragit® L30D55 and Eudragit® FS30D in a ratio of about 1:2% w/w.

In some embodiments, a pharmaceutical composition of the invention comprises pH-dependent, gastrosoluble polymers including, but are not limited to, Eudragit® E100; Eudragit® E12.5; Eudragit® EPO; or combinations thereof.

In some embodiments, a pharmaceutical composition of the invention comprises pH-independent polymers including, but are not limited to, Eudragit® RL100; Eudragit® RLPO; Eudragit® RL30D; Eudragit® RL12.5; Eudragit® RS100; Eudragit® RSPO; Eudragit® RS30 D; Eudragit® RS12.5; Eudragit® NE30D; Eudragit® NE40 D; Eudragit® NM30D; or combinations thereof.

In some embodiments, a pharmaceutical composition of the invention comprises a subcoating. In some embodiments, a subcoating is optional. In some embodiments, subcoating is applied prior to other coatings, including the outer coating or a coating that can control release of the pharmaceutically active agents. In some embodiments, a subcoating is applied so that subsequent coating(s) can be applied uniformly. In some embodiments, a subcoating is applied to provide a uniform release rate of the pharmaceutically active agents. In some embodiments, the subcoating comprises hydroxypropyl methylcellulose (HPMC, also known as hypromellose), hydroxypropylcellulose, polyvinyl alcohol, povidone, copovidone, methylcellulose, hydroxyethyl cellulose, starch, modified starches, sodium carboxymethylcellulose, guar or a combination thereof. In some embodiment, hydroxyethyl cellulose useful for subcoating has sufficiently low molecular weights as to not impede the release of the pharmaceutically active agents. In some embodiments, suitable subcoating material is available under the trademark Opadry® (Colorcon). In some embodiments, Opadry® comprises hypromellose, triacetin, and talc. In some embodiments, subcoating comprises hypromellose (2910) is a blend of Dow Methocel E3 and E6 grades.

In another aspect, in order to reduce or eliminate possible stability issues during storage of the pharmaceutical compositions, the present invention provides for dosage forms that separate the pharmaceutically active agents.

In another aspect, the invention reveals environmental conditions, including temperature, humidity and enclosure specifications that largely maintain the long term stability of each API component of the formulation when prepared as a fixed dose.

### Controlled-Release Formulations

In some embodiments, controlled-release formulations release a pharmaceutically active agent at the same concentration for a certain period of time. A controlled-release formulation can be extended release (XR) or long-acting release (LA), sustained release (SR), delayed or enteric release, repeat action or pulsed release. The controlled-release process can be achieved by multiple mechanisms, out of which two are outstanding: the coating of tablets and beads with polymers or drug particles in capsules, or, alternatively, the dissolution of coating releases the drug over time. The modified-release dosage is a mechanism that (in contrast to immediate-release dosage) delivers a drug with a delay after its administration (delayed-release dosage) or for a prolonged period of time (extended-release [ER, XR, XL] dosage) or to a specific target in the body (targeted-release dosage) (see Yvonne Perrie, Thomas Rades, Pharmaceutics: Drug Delivery and Targeting, Pharmaceutical Press, 2009).

In some embodiments, sustained release dosage forms release pharmaceutically active agent at a predetermined rate in order to maintain a constant drug concentration for a specific period of time with minimum side effects through formulations such as liposomes or drug-polymer conjugates, e.g., hydrogels. In some embodiments, extended-release formulations are sustained-release (SR) or controlled-release (CR). SR maintains drug release over a sustained period but not at a constant rate. CR maintains drug release over a sustained period at a nearly constant rate. In some embodiments, modified-release dosage can allow the pharmaceutically active agent to be released more slowly and steadier into the bloodstream (time-dependent release), which allows it to be administered less frequently than immediate-release (IR) formulations of the same drug. The release in time can be sustained (prolonged release is intended), pulse release, delayed release (e.g., to target different regions of the GI tract), etc. In addition to pills, capsules and injectable drug carriers, other forms of controlled release medicines include gels, implants and devices (e.g., contraceptive implant) and transdermal patches.

### A. Modified Release Formulations

In some embodiments, the pharmaceutical composition of the invention is a modified release formulation. In certain embodiments, the modified release formulation provides for modified release of the statin, the gemcabene or both. In some embodiments, the pharmaceutical composition of the invention comprises from about 0.1 mg to about 80 mg of the statin and from about 150 mg to about 900 mg of gemcabene. In some examples, the pharmaceutical composition comprises from about 10 mg to about 40 mg of the statin and from about 300 mg to about 600 mg of gemcabene, and the gemcabene, the statin, or both gemcabene and statin are in a controlled release form. In those embodiments where both the gemcabene and the statin are in controlled release forms, the controlled release form of gemcabene may be the same as or different from the controlled release form of the statin.

In some embodiments, the pharmaceutical composition is a modified release dosage form comprising from about 0.1 mg to about 80 mg of the statin, from about 150 mg to about 900 mg of gemcabene, and from about 10 mg to 100 mg of a third pharmaceutically active agent wherein the third pharmaceutically active agent is a lipid-lowering agent. In some examples, the pharmaceutical composition comprises from about 10 mg to about 40 mg of the statin and from about 300 mg to about 600 mg of gemcabene, and from about 5 mg to 50 mg of a third pharmaceutically active agent wherein the third pharmaceutically active agent is a lipid modifying agent, anti-fibrolytic agent, or an anti-inflammatory agent; the gemcabene, the statin, the third pharmaceutically active agent or all pharmaceutically active agents are in a controlled release form.

Examples of modified release dosage forms suited for pharmaceutical compositions of the instant invention are described, without limitation, in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; and 6,699,500.

Modified Release Coating

In some embodiments, the pharmaceutical composition of the invention is a modified release formulation comprising a core comprising a pharmaceutically active agent and an outer coating comprising a copolymer. In some embodiments, the copolymer is a methacrylic acid copolymer. In some embodiments, the copolymer is a copolymer comprising methyl acrylate, methyl methacrylate and methacrylic acid repeat units in a ratio of (about 7):(about 3):(about 1), a copolymer comprising methacrylic acid and ethyl acrylate repeat units in a ratio of (about 1):(about 1), or a combination thereof. In some embodiments, the copolymer is selected from Eudragit® L30D55, Eudragit® L100-55, Eudragit® L100, Eudragit® L12.5, Eudragit® S100, Eudragit® S12.5, Eudragit® FS30D, or combinations thereof.

In some embodiments, the outer coating comprises a first copolymer or a second copolymer. In some embodiments, the outer coating comprises a first copolymer and a second copolymer. In some embodiments, the first copolymer is a copolymer comprising methyl acrylate, methyl methacrylate and methacrylic acid repeat units in a ratio of (about 7):(about 3):(about 1). In some embodiments, the first copolymer has a ratio of free carboxyl groups to methyl ester groups of about 1:10. In some embodiments, the first copolymer has a weight average molar mass of about 280,000 g/mol. In some embodiments, the first copolymer is Eudragit® FS30D.

In some embodiments, the second copolymer is a copolymer comprising methacrylic acid and ethyl acrylate repeat units in a ratio of (about 1):(about 1). In some embodiments, the second copolymer has a ratio of free carboxyl groups to ethyl ester groups of about 1:1. In some embodiments, the second copolymer has a weight average molar mass of about 320,000 g/mol. In some embodiments, the second copolymer is Eudragit® L30D55.

In some embodiments, the pharmaceutical composition of the invention, the total amount of the outer coating ranges from about 1% w/w to about 15% w/w of the formulation. In some embodiments, the pharmaceutical composition of the invention, the total amount of the outer coating is about 1%, about 2%, about 3%, about 4%, about 5%, about 6%, about 7%, about 8%, about 9%, about 10%, about 11%, about 12%, about 13%, about 14%, or about 15% w/w of the formulation. In some embodiments, the pharmaceutical composition of the invention is in the form of a tablet comprising an outer coating ranges from about 1% w/w to about 15% w/w of the tablet. In some embodiments, the pharmaceutical composition of the invention is in the form of a tablet comprising an outer coating is about 1%, about 2%, about 3%, about 4%, about 5%, about 6%, about 7%, about 8%, about 9%, about 10%, about 11%, about 12%, about 13%, about 14%, or about 15% w/w of the tablet. In some embodiments, the pharmaceutical composition of the invention is in the form of a tablet comprising an outer coating ranges from about 1% w/w to about 11% w/w of the tablet. In some embodiments, the pharmaceutical composition of the invention is in the form of a tablet comprising an outer coating ranges from about 1% w/w to about 4% w/w of the tablet.

In some embodiments, the pharmaceutical composition of the invention is a modified release formulation comprising a core comprising a pharmaceutically active agent and an outer coating comprising a pH dependent polymer. In some embodiments, the pH dependent polymer is selected from cellulose acetate phthalate (CAS 9004-38-0, dissolves at pH 6), hypromellose acetate succinate (CAS 71138-97-1; dissolution pH range 5.9-7.0), hypromellose phthalate (CAS 9050-31-1; dissolution pH range 5.0-5.5), polyvinyl acetate phthalate (CAS 34481-48-6; dissolution pH range 4.5-5.0), poly-methyl vinyl ether/maleic anhydride (Gantrez®), cellulose acetate trimellitate (CAS 52907-01-4; soluble at pH >5.0), zein (CAS 9010-66-6; soluble at pH >11.5), or a combination thereof.

Modified Release Tablet Comprising a Statin

In some embodiments, the tablet of the invention comprises a) a core comprising a statin or a pharmaceutically acceptable salt thereof and b) an outer coating comprising a copolymer, wherein the core has an outer surface and wherein the outer coating is disposed over the entire outer surface. In some embodiments, the core comprises a therapeutically effective amount of statin or a pharmaceutically acceptable salt thereof.

In some embodiments, the tablet of the invention comprises a) a core comprising a statin or a pharmaceutically acceptable salt thereof and b) an outer coating comprising i) a first copolymer which is a copolymer comprising methyl acrylate, methyl methacrylate and methacrylic acid repeat units in a ratio of (about 7):(about 3):(about 1), or ii) a second copolymer which is a copolymer comprising methacrylic acid and ethyl acrylate repeat units in a ratio of (about 1):(about 1). In some embodiments of the tablet of the invention, the outer coating comprises the first copolymer and the second copolymer.

In some embodiments, the total amount of the first copolymer and the second copolymer ranges from about 1% w/w to about 50% w/w of the tablet. In some embodiments, the total amount of the first copolymer and the second copolymer ranges from about 1% w/w to about 40% w/w of the tablet. In some embodiments, the total amount of the first copolymer and the second copolymer ranges from about 1% w/w to about 30% w/w of the tablet. In some embodiments, the total amount of the first copolymer and the second copolymer ranges from about 1% w/w to about 20% w/w of the tablet. In some embodiments, the total amount of the first copolymer and the second copolymer ranges from about 1% w/w to about 15% w/w of the tablet.

In some embodiments, the total amount of the first copolymer and the second copolymer ranges from about 1% to about 5% w/w of the tablet. In some embodiments, the total amount of the first copolymer and the second copolymer ranges from about 2% to about 3% w/w of the tablet. In some embodiments, the total amount of the first copolymer and the second copolymer ranges from about 2.2% to about 2.8% w/w of the tablet. In some embodiments, the total amount of the first copolymer and the second copolymer ranges from about 2.4% to about 2.6% w/w of the tablet.

In some embodiments, the total amount of the first copolymer and the second copolymer is in an amount of about 1%, about 1.1%, about 1.2%, about 1.3%, about 1.4%, about 1.5%, about 1.6%, about 1.7%, about 1.8%, about 1.9%, 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, or about 5.0% w/w of the tablet. In some embodiments, the total amount of the first copolymer and the second copolymer is in an amount of about 2.2%, about 2.3%, about 2.4%, about 2.5%, about 2.6%, about 2.7%, or about 2.8% w/w of the tablet.

In some embodiments, the tablet of the invention does not comprise the second copolymer. In some embodiments, the amount of the first copolymer ranges from about 1% w/w to about 50% w/w, from about 1% w/w to about 40% w/w, from about 1% w/w to about 30% w/w, from about 1% w/w to about 20% w/w, from about 1% w/w to about 15% w/w, from about 1% to about 5% w/w, from about 2% to about 3% w/w, or from about 2.2% to about 2.8% w/w of the tablet. In some embodiments, the amount of the first copolymer is about 1%, about 1.1%, about 1.2%, about 1.3%, about 1.4%, about 1.5%, about 1.6%, about 1.7%, about 1.8%, about 1.9%, 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, or about 5.0% w/w of the tablet.

In some embodiments, the tablet of the invention does not comprise the first copolymer. In some embodiments, the amount of the second copolymer ranges from about 1% w/w to about 50% w/w, from about 1% w/w to about 40% w/w, from about 1% w/w to about 30% w/w, from about 1% w/w to about 20% w/w, from about 1% w/w to about 15% w/w, from about 1% to about 5% w/w, from about 2% to about 3% w/w, or from about 2.2% to about 2.8% w/w of the tablet. In some embodiments, the amount of the second copolymer is about 1%, about 1.1%, about 1.2%, about 1.3%, about 1.4%, about 1.5%, about 1.6%, about 1.7%, about 1.8%, about 1.9%, 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, or about 5.0% w/w of the tablet.

In some embodiments, the ratio of the first copolymer to the second copolymer ranges from about 95:5 to about 5:95 by weight. In some embodiments, the ratio of the first copolymer to the second copolymer ranges from about 15:1 to about 1:15 by weight. In some embodiments, the ratio of the first copolymer to the second copolymer ranges from about 10:1 to about 1:10 by weight. In some embodiments, the ratio of the first copolymer to the second copolymer ranges from about 5:1 to about 1:5 by weight. In some embodiments, the ratio of the first copolymer to the second copolymer ranges from about 3:1 to about 1:3 by weight. In some embodiments, the ratio of the first copolymer to the second copolymer ranges from about 2:1 to about 1:2 by weight. In some embodiments, the ratio of the first copolymer to the second copolymer is about 2:1 by weight.

In some embodiments, the amount of the first copolymer in the outer coating ranges from about 30 wt % to about 98 wt % of the outer coating. In some embodiments, the amount of the first copolymer in the outer coating ranges from about 40 wt % to about 95 wt % of the outer coating.

In some embodiments, the amount of the second copolymer in the outer coating ranges from about 40 wt % to about 98 wt % of the outer coating. In some embodiments, the amount of the second copolymer in the outer coating ranges from about 50 wt % to about 95 wt % of the outer coating.

In some embodiments, the total amount of the first copolymer and the second copolymer in the outer coating ranges from about 50 wt % to about 99.9 wt % of the outer coating. In some embodiments, the total amount of the first copolymer and the second copolymer in the outer coating ranges from about 60 wt % to about 99.9 wt % of the outer coating. In some embodiments, the total amount of the first copolymer and the second copolymer in the outer coating ranges from about 70 wt % to about 99.9 wt % of the outer coating. In some embodiments, the total amount of the first copolymer and the second copolymer in the outer coating ranges from about 80 wt % to about 99.9 wt % of the outer coating.

In some embodiments, the outer coating is disposed over the outer surface of the core (with or without a subcoat) as an aqueous dispersion or a dispersion in organic solvents comprising a mixture of the first copolymer and the second copolymer. In some embodiments, the outer coating is disposed over the outer surface of the core (with or without a subcoat) at a level ranging from about 1% to about 20% weight gain, relative to core prior to the application of the outer coating. In some embodiments, the outer coating is disposed over the outer surface of the core (with or without a subcoat) at a level ranging from about 2% to about 15% weight gain, relative to core prior to the application of the outer coating. In some embodiments, the outer coating is disposed over the outer surface of the core (with or without a subcoat) at a level ranging from about 3% to about 10% weight gain, relative to core prior to the application of the outer coating. In some embodiments, the outer coating is disposed over the outer surface of the core (with or without a subcoat) at a level ranging from about 5% to about 8% weight gain, relative to core prior to the application of the outer coating. In some embodiments, the outer coating is disposed over the outer surface of the core (with or without a subcoat) at a level of about 10% weight gain (or about 12.6 mg/cm²), relative to core prior to the application of the outer coating. In some embodiments, the outer coating is disposed over the outer surface of the core (with or without a subcoat) at a level of about 7% weight gain (or about 8.9 mg/cm²), relative to core prior to the application of the outer coating.

In some embodiments, the outer coating further comprises one or more pharmaceutically acceptable excipients. In some embodiments, the outer coating further comprises a plasticizer, a detackifier, or a combination thereof. In some embodiments, the outer coating further comprises triethyl citrate or talc. In some embodiments, the outer coating further comprises triethyl citrate and talc. In some embodiments, the outer coating further comprises pre-made glyceryl monostearate dispersion (e.g., PlasACRYL® HTP20; Evonik Corporation). PlasACRYL® is a 20% emulsion containing anti-tacking and plasticizing components that simplify the preparation of a robust coating suspension. PlasACRYL® is commonly used with acrylic polymer coating systems for tablet and multiparticulate coating applications.

In some embodiments, use of pre-made glyceryl monostearate dispersion in the outer coating allows for a thinner outer coating by removal of considerable portion of the coating solids. In some embodiments, the outer coating further comprising pre-made glyceryl monostearate dispersion was applied to a core comprising the statin or a pharmaceutically acceptable salt thereof at a coating level of about 3 to about 4% weight gain (about 3.8 to about 5.1 mg/cm²). In some embodiments, the outer coating further comprising pre-made glyceryl monostearate dispersion was sufficient protection against acidic environment of a stomach an dprovided rapid release of the statin or a pharmaceutically acceptable salt thereof at pH of about 7.0.

In some embodiments of the tablet of the invention, the outer coating is disposed over the outer surface of the core such that the statin or a pharmaceutically acceptable salt thereof in the core is released at a pH ranging from pH 7.0 to pH 7.2.

In some embodiments, the tablet of the invention further comprising a subcoating between the core and the outer coating. In some embodiments, the subcoating comprises neutral pH polymers or pH independent polymers. In some embodiments, the subcoating comprising hydroxypropyl methylcellulose (HPMC), hydroxypropylcellulose, polyvinyl alcohol, povidone, copovidone, methylcellulose, hydroxyethyl cellulose, starch, modified starches, sodium carboxymethylcellulose, guar or combination thereof. In some embodiments, the amount of the subcoating is about 1%, about 1.1%, about 1.2%, about 1.3%, about 1.4%, about 1.5%, about 1.6%, about 1.7%, about 1.8%, about 1.9%, 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, or about 5.0% w/w of the tablet. In some embodiments, the amount of the subcoating is about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, or about 3.5% w/w of the tablet. In some embodiments, the amount of the subcoating is in the range of about 2.0% to about 3.5% w/w of the tablet.

In some embodiments, the subcoating is disposed over the outer surface of the core comprising the statin or a pharmaceutically acceptable salt thereof at a level ranging from about 0.5% to about 20% weight gain relative to the weight of the core prior to the subcoating. In some embodiments, the subcoating is disposed over the outer surface of the core comprising the statin or a pharmaceutically acceptable salt thereof at a level ranging from about 1% to about 15% weight gain relative to the weight of the core prior to the subcoating. In some embodiments, the subcoating is disposed over the outer surface of the core comprising the statin or a pharmaceutically acceptable salt thereof at a level ranging from about 1.5% to about 10% weight gain relative to the weight of the core prior to the subcoating. In some embodiments, the subcoating is disposed over the outer surface of the core comprising the statin or a pharmaceutically acceptable salt thereof at a level ranging from about 2% to about 8% weight gain relative to the weight of the core prior to the subcoating. In some embodiments, the subcoating is disposed over the outer surface of the core comprising the statin or a pharmaceutically acceptable salt thereof at a level ranging from about 2.5% to about 5% weight gain relative to the weight of the core prior to the subcoating. In some embodiments, the subcoating is disposed over the outer surface of the core comprising the statin or a pharmaceutically acceptable salt thereof at a level of about 3% weight gain (or about 3.8 mg/cm²) relative to the weight of the core prior to the subcoating.

In some embodiments, suitable subcoating material is available under the trademark Opadry® (Colorcon). In some embodiments, Opadry® comprises hypromellose, triacetin, and talc. In some embodiments, subcoating comprises hypromellose (2910) is a blend of Dow Methocel E3 and E6 grades. In some embodiments, the subcoat further comprises one or more pharmaceutically acceptable excipients.

In some embodiments, if the statin or a pharmaceutically acceptable salt thereof is alkaline, the subcoat can insulates the outer coating from the alkaline pH of the core. In some embodiments, the subcoat facilitates a more rapid release of the drug substance upon rupture of the outer coating (e.g., enteric coating). In some embodiments, the subcoat is disposed over the outer surface of the core such that the outer coating can be uniformly applied over the subcoated core.

In some embodiments of the tablet of the invention, the outer coating does not comprise the statin or a pharmaceutically acceptable salt thereof. In some embodiments, the subcoat does not comprise the statin or a pharmaceutically acceptable salt thereof. In some embodiment, the outer coating further comprises one or more pharmaceutically acceptable excipients.

In some embodiments, the tablet of the invention comprises the statin or a pharmaceutically acceptable salt thereof in the amount ranging from about 5% to about 95% w/w of the core. In some embodiments, the tablet of the invention comprises the statin or a pharmaceutically acceptable salt thereof in the amount ranging from about 5% to about 75% w/w of the core. In some embodiments, the tablet of the invention comprises the statin or a pharmaceutically acceptable salt thereof in the amount ranging from about 10% to about 50% w/w of the core. In some embodiments, the tablet of the invention comprises the statin or a pharmaceutically acceptable salt thereof in the amount ranging from about 10% to about 15% w/w of the core.

In some embodiments, the tablet of the invention comprises the statin or a pharmaceutically acceptable salt thereof in an amount ranging from about 1% to about 30% w/w of the tablet. In some embodiments, the amount of the statin or pharmaceutically acceptable salt thereof ranges from about 5% to about 20% w/w of the tablet. In some embodiments, the amount of the statin or pharmaceutically acceptable salt thereof is about 5%, about 6%, about 7%, about 8%, about 9%, about 10%, about 11%, about 12%, about 13%, about 14%, about 15%, about 16%, about 17%, about 18%, about 19%, or about 20% w/w of the tablet.

In some embodiments, the tablet of the invention comprises the statin or a pharmaceutically acceptable salt thereof in an amount ranging from about 0.001 mg to about 100 mg. In some embodiments, the tablet of the invention comprises the statin or a pharmaceutically acceptable salt thereof in an amount ranging from about 0.01 mg to about 100 mg. In some embodiments, the tablet of the invention comprises the statin or a pharmaceutically acceptable salt thereof in an amount ranging from about 0.1 mg to about 80 mg. In some embodiments, the tablet of the invention comprises the statin or a pharmaceutically acceptable salt thereof in an amount of about 0.001 mg, about 0.005 mg, about 0.01 mg, about 0.05 mg, about 0.1 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg, about 10 mg, 11 mg, about 12 mg, about 13 mg, about 14 mg, about 15 mg, about 16 mg, about 17 mg, about 18 mg, about 19 mg, about 20 mg, 21 mg, about 22 mg, about 23 mg, about 24 mg, about 25 mg, about 26 mg, about 27 mg, about 28 mg, about 29 mg, about 30 mg, 31 mg, about 32 mg, about 33 mg, about 34 mg, about 35 mg, about 36 mg, about 37 mg, about 38 mg, about 39 mg, about 40 mg, 41 mg, about 42 mg, about 43 mg, about 44 mg, about 45 mg, about 46 mg, about 47 mg, about 48 mg, about 49 mg, about 50 mg, 51 mg, about 52 mg, about 53 mg, about 54 mg, about 55 mg, about 56 mg, about 57 mg, about 58 mg, about 59 mg, about 60 mg, 61 mg, about 62 mg, about 63 mg, about 64 mg, about 65 mg, about 66 mg, about 67 mg, about 68 mg, about 69 mg, about 70 mg, 71 mg, about 72 mg, about 73 mg, about 74 mg, about 75 mg, about 76 mg, about 77 mg, about 78 mg, about 79 mg, about 80 mg, 81 mg, about 82 mg, about 83 mg, about 84 mg, about 85 mg, about 86 mg, about 87 mg, about 88 mg, about 89 mg, about 90 mg, 91 mg, about 92 mg, about 93 mg, about 94 mg, about 95 mg, about 96 mg, about 97 mg, about 98 mg, about 99 mg, or about 100 mg.

In some embodiments, the tablet of the invention comprises the statin or a pharmaceutically acceptable salt thereof in an amount ranging from about 1 mg to about 80 mg per a dosage unit.

In some embodiments, the statin is atorvastatin, simvastatin, pravastatin, rosuvastatin, fluvastatin, lovastatin, dalvastatin, dihydrocompactin, cerivastatin or pitavastatin. In some embodiments, statin is atorvastatin. In some embodiments, the pharmaceutically acceptable salt of the statin is atorvastatin calcium.

In some embodiments, the tablet of the invention is subjected to dissolution testing according to USP <711> Delayed Release Dosage Forms Method A using Apparatus 2 (Paddle Apparatus) at 100 RPM. In some embodiments, no more than 5% of the statin is detected in an acidic dissolution medium after 2 h, wherein the acidic dissolution medium is 0.1N HCl. In some embodiments, no more than 2% of the statin is detected in an acidic dissolution medium after 2 h, wherein the acidic dissolution medium is 0.1N HCl. In some embodiments, 0% of the statin is detected in an acidic dissolution medium after 2 h, wherein the acidic dissolution medium is 0.1N HCl. In some embodiments, the dissolution testing is performed at 37° C.±0.5° C. In some embodiments, the USP <711> Delayed Release Dosage Forms Method A using Apparatus 2 (Paddle Apparatus) at 100 RPM is modified such that the sodium phosphate buffer is added to the dissolution medium to adjust the pH to 7.2.

In some embodiments, the tablet of the invention releases about 20% to about 90% statin in no more than 30 minutes after the pH of the dissolution medium is adjusted to pH 7.2 with sodium phosphate buffer according to USP <711> Delayed Release Dosage Forms Method A using Apparatus 2 (Paddle Apparatus) at 100 RPM. In some embodiments, the tablet releases about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, or about 90% statin in no more than 30 minutes after the pH of the dissolution medium is adjusted to pH 7.2 with sodium phosphate buffer according to USP <711> Delayed Release Dosage Forms Method A using Apparatus 2 (Paddle Apparatus) at 100 RPM.

In some embodiments, the tablet of the invention releases at least about 80% statin in no more than 60 minutes after the pH of a dissolution medium is adjusted to pH 7.2 with sodium phosphate buffer according to USP <711> Delayed Release Dosage Forms Method A using Apparatus 2 (Paddle Apparatus) at 100 RPM. In some embodiments, the tablet of the invention releases at least about 90% statin in no more than 60 minutes after the pH of a dissolution medium is adjusted to pH 7.2 with sodium phosphate buffer according to USP <711> Delayed Release Dosage Forms Method A using Apparatus 2 (Paddle Apparatus) at 100 RPM. In some embodiments, the tablet releases at least about 80%, at least about 81%, at least about 82%, at least about 83%, at least about 84%, at least about 85%, at least about 86%, at least about 87%, at least about 88%, at least about 89%, or at least about 90% statin in no more than 60 minutes after the pH of a dissolution medium is adjusted to pH 7.2 with sodium phosphate buffer according to USP <711> Delayed Release Dosage Forms Method A using Apparatus 2 (Paddle Apparatus) at 100 RPM. In some embodiments, the dissolution testing is performed at 37° C.±0.5° C. In some embodiments, the USP <711> Delayed Release Dosage Forms Method A using Apparatus 2 (Paddle Apparatus) at 100 RPM is modified such that the sodium phosphate buffer is added to the dissolution medium to adjust the pH to 7.2.

In some embodiments, the tablet of the invention releases in the range of about 80% to 100% statin in no more than 60 minutes after the pH of a dissolution medium is adjusted to pH 7.2 with sodium phosphate buffer according to USP <711> Delayed Release Dosage Forms Method A using Apparatus 2 (Paddle Apparatus) at 100 RPM. In some embodiments, the pharmaceutical composition of the invention is a tablet, wherein the tablet releases in the range of about 85% to 100% statin in no more than 60 minutes after the pH of a dissolution medium is adjusted to pH 7.2 with sodium phosphate buffer according to USP <711> Delayed Release Dosage Forms Method A using Apparatus 2 (Paddle Apparatus) at 100 RPM. In some embodiments, the pharmaceutical composition of the invention is a tablet, wherein the tablet releases in the range of about 90% to 100% statin in no more than 60 minutes after the pH of a dissolution medium is adjusted to pH 7.2 with sodium phosphate buffer according to USP <711> Delayed Release Dosage Forms Method A using Apparatus 2 (Paddle Apparatus) at 100 RPM.

United States Pharmacopeia <711> Delayed Release Dosage Forms Apparatus 2 (Paddle Apparatus) Setup: The assembly consists of the following: a vessel, which may be covered, made of glass or other inert, transparent material; a motor; a metallic drive shaft; and a cylindrical basket. The vessel is partially immersed in a suitable water bath of any convenient size or heated by a suitable device such as a heating jacket. The water bath or heating device permits holding the temperature inside the vessel at 37±0.5° C. during the test and keeping the bath fluid in constant, smooth motion. No part of the assembly, including the environment in which the assembly is placed, contributes significant motion, agitation, or vibration beyond that due to the smoothly rotating stirring element. An apparatus that permits observation of the specimen and stirring element during the test is preferable. The vessel is cylindrical, with a hemispherical bottom and with one of the following dimensions and capacities: for a nominal capacity of 1 L, the height is 160 mm to 210 mm and its inside diameter is 98 mm to 106 mm; for a nominal capacity of 2 L, the height is 280 mm to 300 mm and its inside diameter is 98 mm to 106 mm; and for a nominal capacity of 4 L, the height is 280 mm to 300 mm and its inside diameter is 145 mm to 155 mm. Its sides are flanged at the top. A fitted cover may be used to retard evaporation. The shaft is positioned so that its axis is not more than 2 mm at any point from the vertical axis of the vessel and rotates smoothly and without significant wobble that could affect the results. A speed regulating device is used that allows the shaft rotation speed to be selected and maintained at the specified rate given in the individual monograph, within ±4%. Shaft and basket components of the stirring element are fabricated of stainless steel, type 316, or other inert material. A basket having a gold coating of about 0.0001 inch (2.5 μm) thick may be used. A dosage unit is placed in a dry basket at the beginning of each test. The distance between the inside bottom of the vessel and the bottom of the basket is maintained at 25±2 mm during the test. A paddle formed from a blade and a shaft is used as the stirring element. The shaft is positioned so that its axis is not more than 2 mm from the vertical axis of the vessel at any point and rotates smoothly without significant wobble that could affect the results. The vertical center line of the blade passes through the axis of the shaft so that the bottom of the blade is flush with the bottom of the shaft. The distance of 25±2 mm between the bottom of the blade and the inside bottom of the vessel is maintained during the test. The metallic or suitably inert, rigid blade and shaft comprise a single entity. A suitable two part detachable design may be used provided the assembly remains firmly engaged during the test. The paddle blade and shaft may be coated with a suitable coating so as to make them inert. The dosage unit is allowed to sink to the bottom of the vessel before rotation of the blade is started. A small, loose piece of nonreactive material, such as not more than a few turns of wire helix, may be attached to dosage units that would otherwise float.

USP <711> Delayed Release Dosage Forms Method A using Apparatus 2 (Paddle Apparatus) Procedure:

ACID STAGE—Place 750 mL of 0.1 N hydrochloric acid in the vessel, and assemble the apparatus. Allow the medium to equilibrate to a temperature of 37±0.5° C. Place 1 dosage unit in the apparatus, cover the vessel, and operate the apparatus at the specified rate given in the monograph. After 2 hours of operation in 0.1 N hydrochloric acid, withdraw an aliquot of the fluid, and proceed immediately as directed under Buffer Stage. Perform an analysis of the aliquot using a suitable assay method. The procedure is specified in the individual monograph.

BUFFER STAGE—The operations of adding the buffer and adjusting the pH is completed within 5 minutes. With the apparatus operating at the rate specified in the monograph, add to the fluid in the vessel 250 mL of 0.20 M tribasic sodium phosphate that has been equilibrated to 37±0.5° C. Adjust, if necessary, with 2 N hydrochloric acid or 2 N sodium hydroxide to a pH of 6.8±0.05, pH 7.2±0.05 or to a specified pH. Continue to operate the apparatus for 45 minutes, or for the specified time given in the individual monograph. At the end of the time period, withdraw an aliquot of the fluid, and perform the analysis using a suitable assay method. The test may be concluded in a shorter time period than that specified for the Buffer Stage if the requirement for the minimum amount dissolved is met at an earlier time. The aliquot is analyzed using a suitable assay method.

In some embodiments, the USP <711> Delayed Release Dosage Forms Method A using Apparatus 2 (Paddle Apparatus) at 100 RPM is modified such that the sodium phosphate buffer is added to the dissolution medium to adjust the pH to 7.2.

A suitable assay method for detecting statin according to USP <711> Delayed Release Dosage Forms Method A using Apparatus 2 (Paddle Apparatus) includes, but are not limited to, high-performance liquid chromatography or UV/Vis spectrophotometer.

In some embodiments, the tablet of the invention comprising the statin or a pharmaceutically acceptable salt thereof is a microtablet having a diameter ranging from about 1 mm to about 5 mm.

In some embodiments, the tablet of the invention further comprises another pharmaceutically active agent. In some embodiments, the pharmaceutically active agent is in the core with the statin or a pharmaceutically acceptable salt thereof. In some embodiments, the pharmaceutically active agent is not in the core with the statin or a pharmaceutically acceptable salt thereof. In some embodiments, the pharmaceutically active agent is ezetimibe, gemcabene or a pharmaceutically acceptable salt thereof. In some embodiments, the pharmaceutically active agent is gemcabene calcium. In some embodiments, the gemcabene or a pharmaceutically acceptable salt thereof is not coated with the outer coating. In some embodiments, the other pharmaceutically active agent is provided in a therapeutically effective amount.

In some embodiments, the tablet of the invention further comprises two additional pharmaceutically active agents. In some embodiments, the tablet of the invention further comprises ezetimibe or a pharmaceutically acceptable salt thereof and gemcabene or a pharmaceutically acceptable salt thereof. In some embodiments, the two additional pharmaceutically active agents are provided in therapeutically effective amounts.

In some embodiments, the tablet of the invention further comprising another pharmaceutically active agent, wherein the outer coating does not comprise the pharmaceutically acceptable active agent. In some embodiments, the subcoating does not comprise the pharmaceutically acceptable active agent.

In some embodiments of the tablet of the invention, the core comprising the statin or a pharmaceutically acceptable salt thereof is a first core, further comprises a second core, wherein the second core comprises another pharmaceutically active agent. In some embodiments, the pharmaceutically active agent is ezetimibe, gemcabene or a pharmaceutically acceptable salt thereof. In some embodiments, the second core is not coated with the outer coating. In some embodiments, the other pharmaceutically active agent is provided in a therapeutically effective amount.

In some embodiments of the tablet of the invention, the core comprising a first layer and a second layer, wherein the first layer comprises the statin or a pharmaceutically acceptable salt thereof and the second layer comprises another pharmaceutically active agent. In some embodiments, the statin or a pharmaceutically acceptable salt thereof in the first layer do not come in contact with the other pharmaceutically acceptable agent in the second layer. In some embodiments, the first layer and the second layer are separated by a separation coating. In some embodiments, the separation coating comprises hydroxypropyl methylcellulose (HPMC), hydroxypropylcellulose, polyvinyl alcohol or combination thereof.

In some embodiments, the other pharmaceutically active agent is ezetimibe, gemcabene or a pharmaceutically acceptable salt thereof. In some embodiments, the other pharmaceutically active agent is gemcabene calcium.

In some embodiment, an oral dosage form comprising the tablet of invention and a composition comprising another pharmaceutically active agent is provided. In some embodiments, the other pharmaceutically active agent is ezetimibe, gemcabene or a pharmaceutically acceptable salt thereof. In some embodiments, the other pharmaceutically active agent is ezetimibe or gemcabene calcium. In some embodiments, the oral dosage form further comprises an additional pharmaceutically active agent. In some embodiments, the oral dosage form further comprises a separation layer between the tablet and the composition.

In some embodiments of the oral dosage form, the tablet is a first tablet, wherein the oral dosage form comprises a second tablet and the second tablet comprises the other pharmaceutically active agent. In some embodiments, the oral dosage form further comprises a separation layer between the first tablet and the second tablet.

In some embodiments, the separation layer comprises hydroxypropyl methylcellulose (HPMC), hydroxypropylcellulose, polyvinyl alcohol, povidone, copovidone, methylcellulose, hydroxyethyl cellulose, starch, modified starch, sodium carboxymethylcellulose, guar or a combination thereof. In some embodiments, the separation layer further comprises one or more pharmaceutically acceptable excipients.

In some embodiments, the tablet of the invention is a bilayer tablet. In some embodiments, a first layer in the bilayer tablet comprises a statin or a pharmaceutically acceptable salt thereof and a second layer in the bilayer tablet comprises gemcabene or a pharmaceutically acceptable salt thereof. In some embodiments, the first layer in the bilayer tablet is coated by an outer coating comprising a first copolymer or a second copolymer, wherein the first copolymer comprising methyl acrylate, methyl methacrylate and methacrylic acid repeat units in a ratio of (about 7):(about 3):(about 1), and wherein the second copolymer comprising methacrylic acid and ethyl acrylate repeat units in a ratio of (about 1):(about 1). In some embodiment, the second layer in the bilayer tablet does not have the outer coating. In some embodiments, the first layer and the second layer of the bilayer tablet is separated by an inert layer. In some embodiment, the inert layer comprises hydroxypropyl methylcellulose (HPMC), hydroxypropylcellulose, polyvinyl alcohol, povidone, copovidone, methylcellulose, hydroxyethyl cellulose, starch, modified starches, sodium carboxymethylcellulose, guar or combination thereof.

In some embodiments, the pharmaceutical composition of the invention is provided in an oral dosage form. The oral dosage form comprises the tablet of the invention and a composition comprising another pharmaceutically active agent. In some embodiments, the composition comprising another pharmaceutically active agent is compressed around the tablet of the invention. In some embodiments, the composition comprising another pharmaceutically active agent is compressed around the tablet of the invention to surround the entire tablet. In some embodiments, the composition comprising another pharmaceutically active agent is compressed against one side of the tablet of the invention to form a bilayer tablet.

In some embodiments, granules or micro-particulates (e.g. via extrusion spherionization or rotary granulation) of a statin or a pharmaceutically acceptable salt thereof is prepared then coated with the outer coating comprising a first copolymer or a second copolymer, wherein the first copolymer comprising methyl acrylate, methyl methacrylate and methacrylic acid repeat units in a ratio of (about 7):(about 3):(about 1), and wherein the second copolymer comprising methacrylic acid and ethyl acrylate repeat units in a ratio of (about 1):(about 1). In some embodiments, the coated granules or the coated micro-particulates comprising the statin or a pharmaceutically acceptable salt thereof is incorporated into a tableting blend and compressed, optionally with another pharmaceutically active agent. In some embodiments, the compression provides a single layer tablet. In some embodiments, the compression provides a bilayer tablet wherein the coated granules or the coated micro-particulates are in one layer and the other pharmaceutically active agent is in another layer.

In some embodiments, a statin or a pharmaceutically acceptable salt thereof is formulated with the outer coating comprising a first copolymer or a second copolymer, wherein the first copolymer comprising methyl acrylate, methyl methacrylate and methacrylic acid repeat units in a ratio of (about 7):(about 3):(about 1), and wherein the second copolymer comprising methacrylic acid and ethyl acrylate repeat units in a ratio of (about 1):(about 1). In some embodiments, gemcabene or a pharmaceutically acceptable salt thereof is not formulated with the outer coating. In some embodiment, the statin or a pharmaceutically acceptable salt thereof formulated with the outer coating and the gemcabene or a pharmaceutically acceptable salt thereof, formulated without the outer coating is present in one oral dosage form.

In some embodiments of the tablet of the invention, when the tablet is administered to a mammal subject provides, at a time point after the tablet's administration, a lower plasma concentration of total statin lactones than the subject's plasma concentration at the time point after administration of a tablet comprising the statin or a pharmaceutically acceptable salt thereof, but not comprising the outer coating. In some embodiments, the subject is human.

In some embodiments, the tablet of the invention comprising atorvastatin or a pharmaceutically acceptable salt thereof, the tablet when administered to a mammal subject provides, at a time point after the tablet's administration, a lower plasma concentration of total atorvastatin lactone, 2-hydroxyatorvastatin lactone or 4-hydroxyatorvastatin lactone than the subject's plasma concentration at the time point after administration of a tablet comprising a atorvastatin or a pharmaceutically acceptable salt thereof, but not comprising the outer coating (e.g., immediate release tablet). In some embodiments, the subject is human.

In some embodiments, the time point is in a range from about 1 hour to about 24 hours. In some embodiments, the time point is about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, or about 36 hours.

In some embodiments of the tablet of the invention, the statin or a statin metabolite (e.g., statin lactones) are released from the tablet no less than one hour after administration and no more than 4 hours from administration to a subject. In some embodiments, the subject is human. In some embodiments, the statin is atorvastatin. In some embodiments, the pharmaceutically acceptable salt of statin is atorvastatin calcium.

In some embodiments, the tablet of the invention is in a capsule. In some embodiments, the capsule contains the tablet of the invention in the form of microtablets having a diameter ranging from about 1 mm to about 5 mm.

In some embodiments, the capsule further contains gemcabene or a pharmaceutically acceptable salt thereof. In some embodiments, the capsule further contains gemcabene calcium. In some embodiments, the capsule further contains ezetimibe. In some embodiments, the capsule further contains gemcabene or a pharmaceutically acceptable salt thereof and ezetimibe. In some embodiments, the capsule contains the statin in a therapeutically effective amount. In some embodiments, the capsule contains gemcabene or a pharmaceutically acceptable salt thereof or ezetimibe in a therapeutically effective amount.

In some embodiments, the capsule further contains gemcabene or a pharmaceutically acceptable salt thereof in an amount ranging from about 50 mg to about 900 mg per capsule. In some embodiments, the capsule further contains gemcabene or a pharmaceutically acceptable salt thereof in an amount of about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, or an amount ranging from and to any of these values, per capsule.

In some embodiments, the capsule further contains gemcabene or a pharmaceutically acceptable salt thereof in an amount of about 50 mg, about 150 mg, about 300 mg, or about 600 mg per capsule.

In some embodiments, the capsule further contains ezetimibe or a pharmaceutically acceptable salt thereof in an amount ranging from about 1 mg to about 50 mg. In some embodiments, the capsule further contains ezetimibe or a pharmaceutically acceptable salt thereof in an amount of about 1 mg, about 2 mg, about 3 mg, about 4 mg, about 5 mg, about 6 mg, about 7 mg, about 8 mg, about 9 mg, about 10 mg, 11 mg, about 12 mg, about 13 mg, about 14 mg, about 15 mg, about 16 mg, about 17 mg, about 18 mg, about 19 mg, about 20 mg, 21 mg, about 22 mg, about 23 mg, about 24 mg, about 25 mg, about 26 mg, about 27 mg, about 28 mg, about 29 mg, about 30 mg, 31 mg, about 32 mg, about 33 mg, about 34 mg, about 35 mg, about 36 mg, about 37 mg, about 38 mg, about 39 mg, about 40 mg, 41 mg, about 42 mg, about 43 mg, about 44 mg, about 45 mg, about 46 mg, about 47 mg, about 48 mg, about 49 mg, or about 50 mg.

In some embodiments, the present invention provides a kit comprising the tablet of the invention, a pharmaceutical composition comprising gemcabene or a pharmaceutically acceptable salt thereof, and instructions for use of the tablet of the pharmaceutical composition is provided. In some embodiments, the kit comprises gemcabene calcium. In some embodiments, the kit comprises gemcabene in a dosage unit in an amount ranging from 50 mg to about 900 mg. In some embodiments, the kit further comprises an additional pharmaceutically active agent. In some embodiments, the kit further comprises ezetimibe or a pharmaceutically acceptable salt thereof.

In some embodiments of the kit, the pharmaceutical composition is a first pharmaceutical composition and the kit further comprises a second pharmaceutical composition comprising ezetimibe or a pharmaceutically acceptable salt thereof. In some embodiments, the first pharmaceutical composition and the second pharmaceutical composition contains therapeutically effective amount of ezetimibe, gemcabene or a pharmaceutically acceptable salt thereof.

In some embodiments of the present disclosure where a pharmaceutical composition or a kit provides a statin or a pharmaceutically acceptable salt thereof and gemcabene or a pharmaceutically acceptable salt thereof, the two pharmaceutically active agents can be in different dosage form and/or have a different release characteristics. For example, the modified release pharmaceutical composition comprising statin or a pharmaceutically acceptable salt thereof is in a tablet as disclosed herein and gemcabene or a pharmaceutically acceptable salt thereof can be in a separate tablet having different dissolution profile from the tablet comprising statin or a pharmaceutically acceptable salt thereof. In some embodiment of the pharmaceutical combination, the composition comprising statin or a pharmaceutically acceptable salt thereof is in a modified release pharmaceutical composition form and the composition comprising gemcabene or a pharmaceutically acceptable salt thereof is in an immediate release pharmaceutical composition (e.g., no anionic copolymer coatings surrounding gemcabene or a pharmaceutically acceptable salt thereof).

In some embodiments, the tablet of the invention further comprises one or more pharmaceutically acceptable excipients. In some embodiments, any one of the capsules as disclosed herein further comprises one or more pharmaceutically acceptable excipients. In some embodiments, any one of the oral dosage forms as disclosed herein further comprises one or more pharmaceutically acceptable excipients.

### 1. Matrix-Controlled Release

In some embodiments, the pharmaceutical composition is formulated as a matrix-controlled release dosage form. For example, the pharmaceutical composition comprises from about 10 mg to about 40 mg of the statin and from about 300 mg to about 600 mg of gemcabene, wherein the statin, the gemcabene, or both are provided as matrix-controlled release forms. And, in those embodiments comprising matrix-controlled release forms of the statin and the gemcabene, the matrix-controlled release form of the statin may be the same as or different from the matrix-controlled release form of the gemcabene. Suitable matrix-controlled release dosage forms for statins and gemcabene are described, for example, in Takada et al in “Encyclopedia of Controlled Drug Delivery,” Vol. 2, Mathiowitz ed., Wiley, 1999.

In some embodiments, the pharmaceutical composition comprises from about 10 mg to about 40 mg of the statin and from about 300 mg to about 600 mg of gemcabene, wherein the gemcabene comprises a matrix-controlled modified release dosage form. In other embodiments, the pharmaceutical composition comprises from about 10 mg to about 40 mg of the statin and from about 300 mg to about 600 mg of gemcabene, wherein the statin comprises a matrix-controlled modified release dosage form.

In some embodiments, the matrix-controlled release form of the statin, the gemcabene, or both, is formulated as a matrix-controlled release dosage form that comprises an erodible matrix comprising water-swellable, erodible, or soluble polymers, including synthetic polymers, and naturally occurring polymers and derivatives, such as polysaccharides and proteins.

In some embodiments, the erodible matrix of the matrix-controlled release form comprises chitin, chitosan, dextran, or pullulan; gum agar, gum arabic, gum karaya, locust bean gum, gum tragacanth, carrageenans, gum ghatti, guar gum, xanthan gum, or scleroglucan; starches, such as dextrin or maltodextrin; hydrophilic colloids, such as pectin; phosphatides, such as lecithin; alginates; propylene glycol alginate; gelatin; collagen; cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), or ethylhydroxy ethylcellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; or other acrylic acid derivatives, such as homopolymers and copolymers of butylmethacrylate, methylmethacrylate, ethylmethacrylate, ethylacrylate, (2-dimethylaminoethyl)methacrylate, or (trimethylaminoethyl)methacrylate chloride; or any combination thereof.

In another embodiment, the pharmaceutical composition comprises a matrix-controlled modified release form comprising a non-erodible matrix. In some of these embodiments, the statin, the gemcabene, or both, is dissolved or dispersed in an inert matrix and is released primarily by diffusion through the inert matrix once administered. In some embodiments, the non-erodible matrix of the matrix-controlled release form comprises one or more insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinylacetate copolymers, ethylene/propylene copolymers, ethylene/ethyl acrylate copolymers, vinylchloride copolymers with vinyl acetate, vinylidene chloride, ethylene or propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, polyvinyl chloride, plasticized nylon, plasticized polyethyleneterephthalate, natural rubber, silicone rubbers, polydimethylsiloxanes, silicone carbonate copolymers, or hydrophilic polymers, such as ethyl cellulose, cellulose acetate, crospovidone, or cross-linked partially hydrolyzed polyvinyl acetate; fatty compounds, such as carnauba wax, microcrystalline wax, or triglycerides; or any combination thereof.

In a matrix-controlled release system, the desired release kinetics can be controlled, for example, via the polymer type employed, the polymer viscosity, the particle sizes of the polymer and/or the pharmaceutically active agent(s), the ratio of the pharmaceutically active agent(s) versus the polymer, and other excipients in the composition.

The pharmaceutical composition of the instant invention comprising a modified release dosage form may be prepared by methods known to those skilled in the art, including direct compression, dry or wet granulation followed by compression, melt-granulation followed by compression.

### 2. Tablets-In-Capsule System

In some embodiments, the pharmaceutical composition comprises a tablets-in-capsule system. The tablet-in-capsule system is a multifunctional and multiple unit system comprising versatile mini-tablets in a capsule (e.g., a hard gelatin capsule). The mini-tablets may be rapid-release, extended-release, pulsatile, delayed-onset extended-release minitablets, or any combination thereof. In yet another embodiment, combinations of mini-tablets or combinations of mini-tablets and minibeads comprising multiple pharmaceutically active agents may each have specific lag times, of release multiplied pulsatile drug delivery system (DDS), site-specific DDS, slow-quick DDS, quick/slow DDS and zero-order DDS.

In some embodiments, a pharmaceutical composition of the invention is a capsule, wherein the capsule comprises from about 0.1 mg to about 80 mg of a statin or a pharmaceutically acceptable salt thereof and from about 50 mg to about 900 mg of gemcabene or a pharmaceutically acceptable salt thereof; wherein the pharmaceutical composition comprises a plurality of particles of the statin or pharmaceutically acceptable salt thereof and a plurality of particles of the gemcabene or pharmaceutically acceptable salt thereof. In some embodiments, the statin particles, the gemcabene particles, or both, further comprise a binder. In some embodiments, the statin particles, the gemcabene particles, or both, further comprise an extended release coating.

In some embodiments, the pharmaceutical composition is a tablet-in-capsule, wherein the pharmaceutical composition comprises from about 0.1 mg to about 80 mg of a statin or a pharmaceutically acceptable salt thereof and from about 50 mg to about 900 mg of gemcabene or a pharmaceutically acceptable salt thereof. In other embodiments, the pharmaceutical composition comprises at least one addition pharmaceutically active agent. The pharmaceutical composition may comprise from about 5 mg to about 100 mg of the third pharmaceutically active agent or a pharmaceutically acceptable salt thereof. The pharmaceutical composition may comprise at least one pharmaceutically acceptable excipient. The excipient may be a diluent, a disintegrant, a wetting agent, a stabilizing agent, a plasticizer, a coating agent, a film coating agent, a binder, a glidant or a lubricant, or any combination thereof.

In some embodiments, the pharmaceutical composition comprises: a tablet comprising from about 0.1 mg to about 80 mg of a statin or a pharmaceutically acceptable salt thereof and a capsule comprising from about 50 mg to about 900 mg of gemcabene or a pharmaceutically acceptable salt thereof. The capsule comprising the gemcabene may also comprise the tablet comprising the statin.

### 3. Osmotic-Controlled Release Devices

In some embodiments, the pharmaceutical composition comprises from about 0.1 mg to about 80 mg of the statin and from about 50 mg to about 900 mg of gemcabene, wherein the gemcabene, the statin, or both comprises an osmotic-controlled release dosage form.

In some examples, the osmotic-controlled release device comprises a one-chamber system, a two-chamber system, asymmetric membrane technology (AMT), an extruding core system (ECS), or any combination thereof. Generally, such devices have at least two components: (a) the core which contains the pharmaceutically active agent(s); and (b) a semipermeable membrane with at least one delivery port, which encapsulates the core. The semipermeable membrane controls the influx of water to the core from an aqueous environment of use so as to cause drug release by extrusion through the delivery port(s).

In some embodiments, the core of the osmotic device optionally comprises an osmotic agent, which creates a driving force for transport of water from the environment of use into the core of the device. One class of osmotic agents useful in the present invention comprises water-swellable hydrophilic polymers, which are also referred to as “osmopolymers” or “hydrogels,” including, but not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), cross-linked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

Another class of osmotic agents comprises osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol; organic acids, such as ascorbic acid, benzoic acid, fumaric acid, citric acid, maleic acid, sebacic acid, sorbic acid, adipic acid, edetic acid, glutamic acid, p-tolunesulfonic acid, succinic acid, and tartaric acid; urea; and mixtures thereof.

Osmotic agents of different dissolution rates may be employed to influence how rapidly the pharmaceutically active agent(s) is initially delivered from the dosage form. For example, amorphous sugars, such as Mannogeme EZ (SPI Pharma, Lewes, Del.) can be used to provide faster delivery during the first couple of hours (e.g., about 1 to about 5 hrs) to promptly produce the desired therapeutic effect, and gradually and continually release of the remaining amount to maintain the desired level of therapeutic or prophylactic effect over an extended period of time. In this case, the pharmaceutically active agent(s) is released at such a rate to replace the amount of the active ingredient metabolized and excreted by the patient.

The core may also include a wide variety of other excipients and carriers as described herein to enhance the performance of the dosage form or to promote stability or processing.

Materials useful in forming the semipermeable membrane include various grades of acrylics, vinyls, ethers, polyamides, polyesters, and cellulosic derivatives that are water-permeable and water-insoluble at physiologically relevant pHs, or are susceptible to being rendered water-insoluble by chemical alteration, such as crosslinking. Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxlated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

The semipermeable membranes may also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semipermeable membrane may be formed post-coating by mechanical or laser drilling. Delivery port(s) may also be formed in situ by erosion of a plug of water-soluble material or by rupture of a thinner portion of the membrane over an indentation in the core. In addition, delivery ports may be formed during coating process, as in the case of asymmetric membrane coatings of the type disclosed in U.S. Pat. Nos. 5,612,059 and 5,698,220.

The total amount of the pharmaceutically active agent(s) released and the release rate can substantially be modulated via the thickness and porosity of the semipermeable membrane, the composition of the core, and the number, size, and position of the delivery ports.

In some embodiments, the pharmaceutical composition in an osmotic controlled-release dosage form may further comprise additional conventional excipients as described herein to promote performance or processing of the formulation.

The osmotic controlled-release dosage forms can be prepared according to conventional methods and techniques known to those skilled in the art (see, Remington: The Science and Practice of Pharmacy, supra; Santus and Baker, J. Controlled Release 1995, 35, 1-21; Verma et al., Drug Development and Industrial Pharmacy 2000, 26, 695-708; Verma et al., J. Controlled Release 2002, 79, 7-27).

In some embodiments, the pharmaceutical composition provided herein is formulated as AMT controlled-release dosage form that comprises an asymmetric osmotic membrane that coats a core comprising the active ingredient(s) and other pharmaceutically acceptable excipients. See, U.S. Pat. No. 5,612,059 and WO 2002/17918. The AMT controlled-release dosage forms can be prepared according to conventional methods and techniques known to those skilled in the art, including direct compression, dry granulation, wet granulation, and a dip-coating method.

In some embodiments, the pharmaceutical composition provided herein is formulated as ESC controlled-release dosage form that comprises an osmotic membrane that coats a core comprising the pharmaceutically active agent(s), hydroxylethyl cellulose, and other pharmaceutically acceptable excipients.

### 4. Multiparticulate-Controlled Release Devices

In some embodiments, the pharmaceutical composition comprises a modified release dosage form that is fabricated as a multiparticulate-controlled release dosage form that comprises a plurality of particles, granules, or pellets, microparticulates, beads, microcapsules and microtablets, ranging from about 10 μm to about 3 mm, about 50 μm to about 2.5 mm, or from about 100 μm to 1 mm in diameter.

The multiparticulate-controlled release dosage forms can provide a prolonged release dosage form with an improved bioavailability. Suitable carriers to sustain the release rate of pharmaceutically active agent(s) include, without limitation, ethyl cellulose, HPMC, HPMC-phtalate, colloidal silicondioxide and Eudragit®-RSPM.

Pellets suitable to be used in the provided compositions and methods contain 50-80% (w/w) of a drug and 20-50% (w/w) of microcrystalline cellulose or other polymers. Suitable polymers include, but are not limited to, microcrystalline wax, pregelatinized starch and maltose dextrin.

Beads can be prepared in capsule and tablet dosage forms. Beads in tablet dosage form may demonstrate a slower dissolution profile than microparticles in capsule form. Microparticle fillers suitable for compositions and methods of the instant invention include, without limitation, sorbitan monooleate (Span 80), HPMC, or any combination thereof. Suitable dispersions for controlled release latex include, for example, ethyl-acrylate and methyl-acrylate.

In some embodiments, the pharmaceutical composition comprises microcapsules and/or microtablets. In one embodiment, microcapsules comprise extended release polymer microcapsules containing statin and gemcabene with various solubility characteristics. Extended release polymer microcapsules can be prepared with colloidal polymer dispersion in an aqueous environment. In another embodiment, microcapsules suitable for the compositions and methods provided herein can be prepared using conventional microencapsulating techniques (Bodmeier & Wang, 1993).

Such multiparticulates may be made by the processes known to those skilled in the art, including wet- and dry-granulation, extrusion/spheronization, roller-compaction, melt-congealing, and by spray-coating seed cores. See, for example, Multiparticulate Oral Drug Delivery; Marcel Dekker: 1994; and Pharmaceutical Pelletization Technology; Marcel Dekker: 1989. Such materials used to form microparticulates are commercially available, for example, gemcabene is commercially available as Lonza gemcabene granular.

Other excipients as described herein may be blended with the pharmaceutical compositions to aid in processing and forming the multiparticulates. The resulting particles may themselves constitute the multiparticulate dosage form or may be coated by various film-forming materials, such as enteric polymers, pH-dependent polymers, pH-independent polymers, water-swellable, or water-soluble polymers. The multiparticulates can be further processed as a capsule or a tablet.

In other embodiments, the pharmaceutical composition comprises a dosage form that has an instant releasing component and at least one delayed releasing component, and is capable of giving a discontinuous release of the compound in the form of at least two consecutive pulses separated in time from 0.1 hrs to 24 hrs.

In some embodiments, the pharmaceutical composition comprises a capsule encapsulating gemcabene or gemcabene pharmaceutically acceptable salt microparticles covering at least a portion of a surface of an atorvastatin calcium tablet, said tablet comprising

(i) a core comprising about 10 to about 80% atorvastatin calcium, about 15 to about 60% lactose monohydrate, about 10 to about 25% microcrystalline cellulose, 0 to about 10% polyvinylpyrrolidone, 0 to about 10% croscaramellose sodium, 0 to about 10% magnesium stearate;
(ii) a sub coat of about 1% to about 5% weight gain relative to the core eight comprising a suitable excipient such as Opadry or mixtures of suitable excipients; and
(iii) an enteric coating composition applied at about 2% to about 15% weight relative to the core weight, comprising methacrylic acid, methyl acrylate, methyl methacrylate copolymer of about 0% to about 10%, methacrylic acid copolymer type C of about 10% to about 0%, and triethyl citrate of about 0% to about 2%.

In a further embodiment, the gemcabene or gemcabene pharmaceutically acceptable salt microparticles comprise:

a. About 48% to about 50 wt % gemcabene or a pharmaceutically acceptable salt thereof;
b. About 24% to about 26 wt % Lactose Monohydrate;
c. About 1.5% to about 2.5 wt % Hydroxypropylcellulose;
d. About 19% to about 21 wt % Microcrystalline Cellulose;
e. About 2% to about 4 wt % Croscarmellose Sodium; and

About 0.4% to about 0.6 wt % Magnesium stearate.

In another embodiment, the atorvastatin calcium tablet core comprises:

a. about 13% to about 14 wt % atorvastatin calcium;
b. about 39% to about 41 wt % lactose monohydrate;
c. about 22% to about 23 wt % calcium carbonate
d. about 18% to about 20 wt % microcrystalline cellulose;
e. about 1.5% to about 2.5 wt % polyvinylpyrrolidone;
f. about 0.2% to about 0.3 wt % polysorbate 80;
g. about 2% to about 3 wt % croscaramellose sodium; and
h. about 0.3% to about 0.5 wt % magnesium stearate.

### B. Oral Administration

The pharmaceutical compositions provided herein may comprise solid, semisolid, gelmatrix or liquid dosage forms for oral administration. As used herein, oral administration also include buccal, lingual, and sublingual administration. Suitable oral dosage forms include, without limitation, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, syrups or any combination thereof. In addition to the pharmaceutically active agents, the pharmaceutical composition may contain one or more pharmaceutically acceptable carriers or excipients, including, but not limited to, binders, fillers, diluents, disintegrants, wetting agents, lubricants, glidants, coloring agents, dye-migration inhibitors, sweetening agents, and flavoring agents.

In some embodiments, the pharmaceutical composition of the invention is a tablet. In some instances, the tablet comprises one or more excipients. An excipient may be a diluent, a disintegrant, a wetting agent, a swellable agent, a binder, a glidant, a lubricant, a coating vehicle (e.g, a film coating vehicle), an anti-foaming agent, a stabilizing agent or any combination thereof. For example, the tablet comprises a binder (e.g., microcrystalline cellulose, dibasic calcium phosphate, sucrose, corn starch, polyvinylpyrridone, hydroxyporopyl cellulose, hydroxymethyl cellulose, or any combination thereof). In another example, the tablet comprises a disintegrant. The tablet may comprise a disintegrant such as sodium croscarmellose or sodium starch glycolate, or combinations of disintegrants. In other examples, the tablet comprises a lubricant (e.g., stearic acid as free acid or as a salt, magnesium stearate, sodium stearyl fumarate, hydrogenated oils, or colloidal silicon dioxide, or any combination thereof).

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof.

Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The binder or filler may be present from about 5 to about 49% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as various types of sodium starch glycolate; polacrilin potassium; starches, such as corn starch, potato starch, tapioca starch, and pre-gelatinized starch; clays; aligns; and mixtures thereof. The amount of disintegrant in the pharmaceutical compositions provided herein varies upon the type of formulation, and is readily discernible to those of ordinary skill in the art. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W. R. Grace Co., Baltimore, Md.) and CAB-O-SIL® (Cabot Co. of Boston, Mass.); and mixtures thereof. The pharmaceutical compositions provided herein may contain about 0.1 to about 5% by weight of a lubricant.

Suitable glidants include colloidal silicon dioxide, CAB-O-SIL® (Cabot Co. of Boston, Mass.), and asbestos-free talc.

Coloring agents include any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye.

Flavoring agents include natural flavors extracted from plants, such as fruits, and synthetic blends of compounds which produce a pleasant taste sensation, such as peppermint and methyl salicylate.

Sweetening agents include sucrose, lactose, mannitol, syrups, glycerin, sucralose, and artificial sweeteners, such as saccharin and aspartame.

Suitable emulsifying agents include gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suspending and dispersing agents include sodium carboxymethylcellulose, pectin, tragacanth, Veegum, acacia, sodium carbomethylcellulose, hydroxypropyl methylcellulose, and polyvinylpyrolidone. Preservatives include glycerin, methyl and propylparaben, benzoic add, sodium benzoate and alcohol. Wetting agents include propylene glycol monostearate, sorbitan monooleate, diethylene glycol monolaurate, and polyoxyethylene lauryl ether.

Solvents include glycerin, sorbitol, ethyl alcohol, and syrup.

Examples of non-aqueous liquids utilized in emulsions include mineral oil and cottonseed oil. Organic acids include citric and tartaric acid. Sources of carbon dioxide include sodium bicarbonate and sodium carbonate.

It should be understood that many carriers and excipients may serve several functions, even within the same formulation.

The pharmaceutical compositions provided herein may be provided as compressed tablets, tablet triturates, chewable lozenges, rapidly dissolving tablets, multiple compressed tablets, enteric-coating tablets, sugar-coated tablets, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, methacrylic acid copolymers, methacrylic and methacrylate acid copolymers, fatty acids, fats, phenylsalicylate, waxes, shellac, ammoniated shellac, cellulose acetate phthalates, cellulose acetate butyrate, hydroxypropylmethylcellulose phthalate (HPMCP), and algenic acid salts such as sodium or potassium alginate. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The pharmaceutical compositions provided herein may be soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

The pharmaceutical compositions provided herein may be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquids or solvent, emulsifying agent, and preservative. Suspensions may include a pharmaceutically acceptable suspending agent and preservative. Aqueous alcoholic solutions may include a pharmaceutically acceptable acetal, such as a di(lower alkyl)acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

The pharmaceutical compositions provided herein for oral administration may be also provided in the forms of liposomes, micelles, microspheres, multiparticulate-filled capsules (enterically-coated microbeads in a capsule) or nanosystems. Micellar dosage forms can be prepared as described in U.S. Pat. No. 6,350,458.

The pharmaceutical compositions provided herein may be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the above dosage forms. And, flavoring and sweetening agents are especially useful in the formation of chewable tablets and lozenges.

The pharmaceutical compositions provided herein may be formulated as immediate or modified release dosage forms, including delayed-, extended, pulsed-, controlled, targeted-, and programmed-release forms.

The pharmaceutical compositions provided herein may be co-formulated with other active ingredients which do not impair the desired therapeutic action, or with substances that supplement the desired action.

The tablet dosage forms may be prepared from the active ingredient in powdered, crystalline, or granular forms, alone or in combination with one or more carriers or excipients described herein, including binders, disintegrants, controlled-release polymers, lubricants, diluents, and/or colorants.

In some aspects, the present invention provides a pharmaceutical composition in the form of a tablet, wherein the tablet comprises from about 0.1 mg to about 80 mg of a statin or a pharmaceutically acceptable salt thereof; from about 50 mg to about 900 mg of gemcabene or a pharmaceutically acceptable salt thereof; and one or more excipients.

In some embodiments, the pharmaceutical composition is in the form of a tablet and the tablet comprises from about 0.1 mg to about 80 mg of a statin or a pharmaceutically acceptable salt thereof; from about 50 mg to about 900 mg of gemcabene or a pharmaceutically acceptable salt thereof; and one or more excipients selected from a diluent, a disintegrant, a wetting agent, a binder, a glidant, a lubricant, or any combination thereof. For example, the tablet comprises a binder. And, in some instances, the binder comprises microcrystalline cellulose, dibasic calcium phosphate, sucrose, corn starch, polyvinylpyrridone, hydroxypropyl cellulose, hydroxymethyl cellulose, or any combination thereof. In another example, the tablet comprises a disintegrant. In some instances, the disintegrant comprises sodium croscarmellose, sodium starch glycolate, or any combination thereof. In other examples, the tablet comprises a lubricant. And, in some instances, the lubricant comprises magnesium stearate stearic acid, hydrogenated oil, sodium stearyl fumarate, or any combination thereof.

In one embodiment, the tablet comprises from about 10 mg to about 40 mg of the statin and from about 150 mg to about 600 mg of gemcabene. In another embodiment the tablet comprises from about 10 mg to about 40 mg of the statin and from about 150 mg to about 300 mg of gemcabene. In some of these examples, the statin is the calcium salt of atorvastatin. In other examples, the gemcabene is the calcium salt of gemcabene. And, in some examples, the tablet further comprises calcium carbonate, potassium carbonate, or a combination thereof.

In some embodiments, the tablet comprises from about 10 to about 60 mg of a statin.

In some embodiments, the statin is atorvastatin, simvastatin, pravastatin, rivastatin, mevastatin, fluindostatin, velostatin, fluvastatin, dalvastatin, dihydrocompactin, compactin, cerivastatin, or lovastatin, or any pharmaceutically acceptable salts thereof. For example, the statin is atorvastatin, simvastatin, or pharmaceutically acceptable salts thereof. In other examples, the statin is a calcium salt of atorvastatin.

In some embodiments, the tablet comprises a binder such as any of the binders described herein.

In some embodiments, the tablet comprises a disintegrant such as any of the disintegrants described herein.

In some embodiments, the tablet comprises a lubricant such as any of the lubricants described herein.

In some embodiments, the gemcabene is the calcium salt of gemcabene.

In some embodiments, the pharmaceutical composition further comprises calcium carbonate, potassium carbonate, or a combination thereof.

### C. Kits

Another aspect of the present invention provides kits comprising the pharmaceutical composition of the invention. In some embodiments, the kits comprise instructions that instruct a use of the pharmaceutical composition of the invention.

In one embodiment, the present invention provides kits comprising a capsule which comprises a first single dose formulation comprising from about 0.1 mg to about 80 mg of a statin or a pharmaceutically acceptable salt thereof and a second single dose formulation comprising from about 50 mg to about 900 mg of gemcabene or a pharmaceutically acceptable salt thereof and instructions for the use thereof. In some embodiments, the kit comprises a capsule which comprises a first single dose formulation comprising from about 10 mg to about 60 mg of a statin or a pharmaceutically acceptable salt thereof and a second single dose formulation comprising from about 150 mg to about 600 mg of gemcabene or a pharmaceutically acceptable salt thereof and instructions for the use thereof. In other embodiments, the kit comprises a capsule which comprises a first single dose formulation comprising from about 10 mg to about 40 mg of a statin or a pharmaceutically acceptable salt thereof and a second single dose formulation comprising from about 150 mg to about 450 mg of gemcabene or a pharmaceutically acceptable salt thereof and instructions for the use thereof. In still other embodiments, the kit comprises a capsule which comprises a first single dose formulation comprising from about 10 mg to about 60 mg of a statin or a pharmaceutically acceptable salt thereof and a second single dose formulation comprising from about 50 mg to about 300 mg of gemcabene or a pharmaceutically acceptable salt thereof and instructions for the use thereof. In some embodiments, the present invention provides a kit comprising a single dose formulation comprising from about 0.1 mg to about 80 mg of a statin or a pharmaceutically acceptable salt thereof and from about 150 mg to about 900 mg of gemcabene or a pharmaceutically acceptable salt thereof; and instructions for the use thereof. The single dose combinations provided in the kit may be, for example, microtablets, microbeads, microbeads in a capsule or capsules.

In some embodiments, the first single dose formulation and the second single dose formulation are in separate containers (e.g., beads or microparticles prepared or formulated separately and stored in capsules). In some embodiments, the first single dose formulation and the second single dose formulation are in the same container, such as a capsule. In some embodiments, the first single dose formulation and the second single dose formulation are stored in different compartments of the container, and each formulation may have a different release profile.

In yet another embodiment, the kit comprises a third pharmaceutically active agent. In some embodiments, the third pharmaceutically active agent is a lipid-reducing agent. In any of the above embodiments of a single dose formulation including a third pharmaceutically active agent, the fixed dose combination is provided in a single container, such as a capsule or a tablet.

Another aspect of the present invention provides a kit comprising a first single dose formulation comprising from about 50 mg to about 60 mg of a statin and a second single dose formulation comprising from about 50 mg to about 900 mg of gemcabene; and instructions for the use thereof.

In some embodiments, the first single dose formulation and the second single dose formulation are stored in separate containers.

In some embodiments, the first single dose formulation and the second single dose formulation are stored in the same container.

In some embodiments, the container is a capsule, a tablet, a bottle, a vial, a blister pack, or any combination thereof.

Another aspect of the present invention provides a kit comprising a single dose formulation comprising from about 10 mg to about 40 mg of a statin and from about 300 mg to about 600 mg of gemcabene; and instructions for the use thereof.

In some embodiments, the statin is atorvastatin, simvastatin, pravastatin, rosuvastatin, fluvastatin, lovastatin, pitavastatin or any pharmaceutically acceptable salt thereof. In some embodiments, the statin is atorvastatin or a pharmaceutically acceptable salt thereof.

In some embodiments, the single dose formulation further comprises a tablet.

In some embodiments, the tablet comprises one or more excipients (e.g., a diluent, a disintegrant, a wetting agent, a binder, a glidant, a lubricant, or any combination thereof).

### Methods for Treatment or Prevention

The present invention provides methods for treating or preventing a disorder of lipoprotein metabolism, comprising administering to a subject in need thereof an effective amount of a composition of the invention. In some embodiments, the methods of the invention are used to treat or prevent a disorder of lipoprotein metabolism in a subject in need thereof, without inducing hepatotoxicity or a musculoskeletal disorder.

In some embodiments, present invention provides methods for treating or preventing musculoskeletal discomfort in a subject being administered a statin, comprising administering to a subject in need thereof an effective amount of a composition of the invention. In some embodiments, the musculoskeletal discomfort is caused by myalgia or myositis. In some embodiments, the musculoskeletal discomfort is caused by the conversion of the acid form of the statin into the lactone form of the statin.

Examples of disorders of lipoprotein metabolism include, but are limited to, dyslipidemia, dyslipoproteinemia, mixed dyslipidemia, atherosclerotic cardiovascular disease (ASCVD), type IIb hyperlipidemia or familial combined hyperlipidemia, familial hypercholesterolemia, familial chylomicronemia syndrome, hypertriglycerdemia, dysbetalipoproteinemia, lipoprotein overproduction or deficiency, elevation of total cholesterol, elevation of low density lipoprotein cholesterol concentration, elevation of very low density lipoprotein cholesterol concentration, elevation of non-high-density lipoprotein (non-HDL) cholesterol concentration, elevation of apolipoprotein B level, elevation of apolipoprotein C-III level, elevation of C-reactive protein level, elevation of fibrinogen level, elevation of lipoprotein(a) level, increased risk of thrombosis, increased risk of a blood clot, low high-density lipoprotein (HDL)-cholesterol level, elevation of low density lipoprotein concentration, elevation of very low density lipoprotein concentration, elevation of triglyceride concentration, prolonged post-prandial lipemia, lipid elimination in bile, metabolic disorder, phospholipid elimination in bile, oxysterol elimination in bile, abnormal bile production, peroxisome proliferator activated receptor-associated disorder, hypercholesterolemia, hyperlipidemia and visceral obesity.

In some embodiments, the disorder of lipoprotein metabolism is dyslipidemia, dyslipoproteinemia, mixed dyslipidemia, atherosclerotic cardiovascular disease (ASCVD), type IIb hyperlipidemia, familial combined hyperlipidemia, familial hypercholesterolemia, familial chylomicronemia syndrome, hypertriglycerdemia, dysbetalipoproteinemia, metabolic syndrome, lipoprotein overproduction, lipoprotein deficiency, non-insulin dependent diabetes, abnormal lipid elimination in bile, a metabolic disorder, abnormal phospholipid elimination in bile, an abnormal oxysterol elimination in bile, an abnormal bile production, a peroxisome proliferator activated receptor-associated disorder, hypercholesterolemia, hyperlipidemia or visceral obesity. In other embodiments, the disorder of lipoprotein metabolism is mixed dyslipidemia, atherosclerotic cardiovascular disease (ASCVD), type IIb hyperlipidemia, familial combined hyperlipidemia, or familial hypercholesterolemia. In some embodiments, the disorder of lipoprotein metabolism is hypertriglyceridemia. In other embodiments, the hypertriglyceridemia is a severe hypertriglyceridemia. “Severe hypertriglyceridemia” may be defined as a subject having a baseline plasma triglyceride level of greater than or equal to 500 mg/dl.

The present invention further provides methods for reducing a subject's plasma triglyceride level, comprising administering to a subject in need thereof an effective amount of a composition of the invention. In some embodiments, a subject has a plasma triglyceride level greater than 150 mg/dl before the onset of treatment.

The present invention further provides methods for reducing a subject's total cholesterol level, low density lipoprotein cholesterol concentration, low density lipoprotein concentration, very low density lipoprotein cholesterol concentration, very low density lipoprotein concentration, non-HDL cholesterol concentration, non-HDL concentration, apolipoprotein B level, triglyceride concentration, apolipoprotein C-III level, C-reactive protein level, fibrinogen level, or lipoprotein(a) level in the subject's blood plasma or serum, comprising administering to a subject in need thereof an effective amount of a composition of the invention.

The present invention further provides methods for lowering a subject's low-density lipoprotein cholesterol (LDL-C) level, comprising administering to a subject in need thereof an effective amount of a composition of the invention, wherein the subject is on a stable dose of a statin.

The present invention provides methods for elevating a subject's high density lipoprotein cholesterol concentration, high density lipoprotein concentration, or apolipoprotein A-I level in the subject's blood plasma or serum, comprising administering to a subject in need thereof an effective amount of a composition of the invention.

The present invention provides methods for reducing a subject's risk of developing a disorder or developing a condition, comprising administering to a subject in need thereof an effective amount of a composition of the invention, wherein the disorder or a condition is thrombosis, blood clot, primary cardiovascular event, secondary cardiovascular event, progression to nonalcoholic fatty liver disease, nonalcoholic steatohepatitis, liver cirrosis, hepatocellular carcinoma, liver failure, pancreatitis, or pulmonary fibrosis. In some embodiments, the disorder or the condition is pancreatitis.

The present invention further provides methods for treating or preventing ballooning or inflammation in the liver of a subject, comprising administering to a subject in need thereof an effective amount of a composition of the invention. In some embodiments, treating or preventing ballooning or inflammation in the liver of a subject is reducing ballooning or inflammation in the liver of a subject.

The present invention provides methods for treating or preventing post-prandial lipemia, comprising administering to a subject in need thereof an effective amount of a composition of the invention.

The present invention provides methods for treating or preventing hypoalphalipoproteinemia, comprising administering to a subject in need thereof an effective amount of a composition of the invention.

The present invention provides methods for reducing a magnitude or duration of post-prandial lipemia, comprising administering to a subject in need thereof an effective amount of a composition of the invention.

The present invention provides methods for reducing a fat content of the liver of a subject, comprising administering to a subject in need thereof an effective amount of a composition of the invention.

The present invention further provides methods for reducing a subject's risk of thrombosis or blood clot, comprising administering to a subject in need thereof an effective amount of a composition of the invention.

In some embodiments, the methods of the invention are effective to reduce a subject's plasma triglyceride level to below about 200 mg/dl or to below about 150 mg/dl. In some embodiments, the methods of the invention are effective to reduce a subject's plasma triglyceride level to below about 200 mg/dl or to below about 150 mg/dl within about 8 to about 12 weeks after administering a compound of the invention.

In some embodiments, the methods of the invention are effective to reduce the subject's plasma triglyceride level by at least 10% in a subject whose baseline plasma triglyceride level is 500 mg/dl or higher, comprising administering to a subject in need thereof an effective amount of a composition of the invention. In some embodiments, the methods of the invention are effective to reduce the subject's plasma triglyceride level by at least 10%, at least 15%, at least 20%, at least 25%, at least 30%, at least 35%, at least 40%, at least 45%, at least 50%, at least 55%, at least 60%, or any range between any of these values, of the baseline plasma triglyceride level where the subject has a baseline plasma triglyceride level of 500 mg/dl or higher. In some embodiments, the methods of the invention are effective to reduce the subject's plasma triglyceride level by up to about 60% of the baseline plasma triglyceride level in a subject whose baseline plasma triglyceride level is 500 mg/dl or higher, comprising administering to a subject in need thereof an effective amount of a composition of the invention.

In some embodiments, the methods of the invention are effective to reduce the subject's plasma triglyceride level by at least 10% in a subject whose baseline plasma triglyceride level is 200 mg/dl or higher, comprising administering to a subject in need thereof an effective amount of a composition of the invention. In some embodiments, the methods of the invention are effective to reduce the subject's plasma triglyceride level by at least 10%, at least 15%, at least 20%, at least 25%, at least 30%, at least 35%, at least 40%, or any range between any of these values, of the baseline plasma triglyceride level where the subject has a baseline plasma triglyceride level is 200 mg/dl or higher. In some embodiments, the methods of the invention are effective to reduce the subject's plasma triglyceride level by up to about 35%, by up to about 36%, by up to about 37%, by up to about 38%, by up to about 39%, or by up to about 40% of the baseline plasma triglyceride level in a subject whose baseline plasma triglyceride level is 200 mg/dl or higher, comprising administering to a subject in need thereof an effective amount of a composition of the invention.

The present invention further provides methods for reducing a subject's plasma LDL cholesterol level, comprising administering to a subject in need thereof an effective amount of a composition of the invention.

In some embodiments, the subject's plasma LDL cholesterol level is reduced to below about 130 mg/dl. In some embodiments, the subject's plasma LDL cholesterol level is reduced to below about 130 mg/dl within about 8 to about 12 weeks of administering a composition of the invention.

The present invention further provides methods for reducing a subject's ApoB level, comprising administering to a subject in need thereof an effective amount a composition of the invention. In some embodiments, the subject's ApoB level is reduced to below about 120 mg/dl. In some embodiments, the subject's ApoB level is reduced to below about 120 mg/dl within about 8 to about 12 weeks following administering a composition of the invention.

In some embodiments, the subject has metabolic syndrome, type-2 diabetes, impaired glucose tolerance, obesity, dyslipidemia, hepatitis B, hepatitis C, a human immunodeficiency virus (HIV) infection, or a metabolic disorder such as Wilson's disease, a glycogen storage disorder, galactosemia, an inflammatory condition or an elevated body mass index above what is normal for gender, age and height. Without being bound by theory, metabolic syndrome, type-2 diabetes, impaired glucose tolerance, obesity, dyslipidemia, hepatitis B, hepatitis C, an HIV infection, or a metabolic disorder such as Wilson's disease, a glycogen storage disorder or galactosemia is believed to be a risk factor for developing fatty liver (steatosis).

In some embodiments, the subject has an HIV infection and the subject is being treated with a highly active antiretroviral therapy (HAART) agent such as an antiretroviral inhibitor. Without being bound by theory, a composition of the invention is catabolized to a much lesser extent by the same P450 enzymes which metabolizes the antiretroviral inhibitors when treating an HIV patient undergoing a antiretroviral inhibitor treatment.

The present invention further provides methods for treating or preventing a disorder of glucose metabolism, comprising administering to a subject in need thereof an effective amount of a composition of the invention.

Examples of disorders of glucose metabolism include, but are not limited to, is insulin resistance, impaired glucose tolerance, impaired fasting glucose (levels in blood), diabetes mellitus, lipodystrophy, familial partial lipodystrophy, obesity, peripheral lipoatrophy, diabetic nephropathy, diabetic retinopathy, renal disease, and septicemia. In some embodiments, obesity is central obesity.

The present invention further provides methods for treating or preventing a cardiovascular disorder or a related vascular disorder, comprising administering to a subject in need thereof an effective amount of a composition of the invention.

Examples of cardiovascular disorders or a related vascular disorders include, but are not limited to, atherosclerosis, hypertension, coronary artery disease, peripheral vascular disease, myocardial infarction, arrhythmia, atrial fibrillation, heart valve disease, heart failure, cardiomyopathy, myopathy, pericarditis, impotence, and thrombotic disorder.

The present invention further provides methods for reducing a subject's risk of having an adverse cardiovascular or vascular event, comprising administering to a subject in need thereof an effective amount of a composition of the invention.

In some embodiments, the cardiovascular or vascular event is a primary cardiovascular event. In other embodiments, the cardiovascular event is a secondary cardiovascular event. Examples of cardiovascular events include, but are not limited to, myocardial infarction, stroke angina, acute coronary syndrome, coronary artery bypass graft surgery and cardiovascular death. A primary cardiovascular event is the first cardiovascular event that a patient experiences. If the same patient experiences a subsequent cardiovascular event, then the subsequent cardiovascular event is a secondary cardiovascular event.

The present invention further provides methods for treating or preventing liver disease or an abnormal liver condition, comprising administering to a subject in need thereof an effective amount of a composition of the invention.

Examples of liver disease or liver conditions include, but are not limited to, nonalcoholic fatty liver disease (NAFLD), nonalcoholic steatohepatitis (NASH), alcoholic steatohepatitis, cirrhosis, inflammation, liver fibrosis, partial fibrosis, primary biliary cirrhosis, primary sclerosing cholangitis, liver failure, hepatocellular carcinoma (HCC), liver cancer, hepatic steatosis, hepatocyte ballooning (also known as hepatocellular ballooning), hepatic lobular inflammation, and hepatic triglyceride accumulation. In some embodiments, the liver disease or the liver condition is NAFLD or NASH. In some embodiments, the liver disease or the liver condition is NAFLD. In other embodiments, the liver disease or the liver condition is NASH. In some embodiments, the liver disease or the liver condition is hepatic steatosis. In some embodiments, the liver disease or the liver condition is liver fibrosis.

In some embodiments, treating or preventing liver fibrosis, NAFLD, or NASH includes regressing, stabilizing, or inhibiting progression of liver fibrosis, NAFLD, or NASH.

The present invention further provides methods for reducing liver fat (fat content of the liver), stabilizing the amount of liver fat, or reducing the accumulation of liver fat, comprising administering to a subject in need thereof an effective amount of a composition of the invention.

The present invention further provides methods for treating or preventing lobular inflammation or hepatocyte ballooning, comprising administering to a subject in need thereof an effective amount of a composition of the invention. In some embodiments, treating or preventing lobular inflammation or hepatocyte ballooning is slowing the progression of, stabilizing, or reducing the lobular inflammation or hepatocyte ballooning.

The present invention further provides methods for treating or preventing a disease caused by an increased level of fibrosis, comprising administering to a subject in need thereof an effective amount of a composition of the invention. In some embodiments, the disease caused by an increased level of fibrosis is a lung disease. Examples of diseases caused by an increased level of fibrosis include, but are not limited to, chronic obstructive pulmonary disease, cystic fibrosis, idiopathic pulmonary fibrosis, emphysema, nephrogenic fibrosis, endometrial fibrosis, perineural fibrosis, hepatic fibrosis, myocardial fibrosis, acute lung injury, radiation-induced lung injury following treatment for cancer, progressive massive fibrosis, a complication of coal workers' pneumoconiosis (lungs), cirrhosis (liver), atrial fibrosis, endomyocardial fibrosis, old myocardial infarction, arterial stiffness (heart), glial scar (brain), arthrofibrosis (knee, shoulder, other joints), Crohn's Disease (intestine), Dupuytren's contracture (hands, fingers), keloid (skin), mediastinal fibrosis (soft tissue of the mediastinum), myelofibrosis (bone marrow), Peyronie's disease (penis), nephrogenic systemic fibrosis (skin), retroperitoneal fibrosis (soft tissue of the retroperitoneum), scleroderma/systemic sclerosis (skin, lungs), and some forms of adhesive capsulitis (shoulder). In some embodiments, the disease caused by increased levels of fibrosis is a chronic obstructive pulmonary disease or an idiopathic pulmonary fibrosis.

The present invention further provides methods for treating or preventing a disease associated with increased inflammation, comprising administering to a subject in need thereof an effective amount of a composition of the invention. In some embodiments, the disease associated with increased inflammation is an autoimmune disease.

Examples of diseases associated with increased inflammation include, but are not limited to, multiple sclerosis, inflammatory bowel disease, celiac disease, Crohn's disease, antiphospholipid syndrome, atherosclerosis, autoimmune encephalomyelitis, autoimmune hepatitis, Graves' disease, ulcerative colitis, multiple sclerosis, myasthenia gravis, myositis, polymyositis, Raynaud's phenomenon, rheumatoid arthritis, scleroderma, Sjogren's syndrome, systemic lupus, type 1 diabetes and uveitis. In some embodiments, the disease associated with increased inflammation is multiple sclerosis, inflammatory bowel disease, celiac disease, or Crohn's disease.

The present invention further provides methods for preventing death from or increasing survival from a disease associated with increased inflammation, comprising administering to a subject in need thereof an effective amount of a composition of the invention. In some embodiments, the disease associated with increased inflammation is influenza, sepsis, or a viral disease.

Examples of viral diseases include, but are not limited to, influenza, human immunodeficiency virus infection, hepatitis B, and hepatitis C.

The present invention further provides methods for treating or preventing a C-reactive protein-related disorder, comprising administering to a subject in need thereof an effective amount of a composition of the invention.

Examples of C-reactive protein related disorders include, but are not limited to, inflammation, ischemic necrosis, and a thrombotic disorder.

The present invention further provides methods for treating or preventing a sulfatase-2-related disorder, comprising administering to a subject in need thereof an effective amount of a composition of the invention.

The present invention further provides methods for treating or preventing an apolipoprotein C-III-related disorder, comprising administering to a subject in need thereof an effective amount of a composition of the invention.

The present invention further provides methods for treating or preventing Alzheimer's disease, comprising administering to a subject in need thereof an effective amount of a composition of the invention.

The present invention further provides methods for treating or preventing Parkinson's disease, comprising administering to a subject in need thereof an effective amount of a composition of the invention.

The present invention further provides methods for treating or preventing pancreatitis, comprising administering to a subject in need thereof an effective amount of a composition of the invention.

The present invention further provides methods for treating or preventing the risk of developing pancreatitis, comprising administering to a subject in need thereof an effective amount of a composition of the invention.

The present invention further provides methods for treating or preventing a pulmonary disorder, comprising administering to a subject in need thereof an effective amount of a composition of the invention. In some embodiments, the pulmonary disorder is a chronic obstructive pulmonary disease or an idiopathic pulmonary fibrosis.

The present invention further provides methods for treating or preventing musculoskeletal discomfort, comprising administering to a subject in need thereof an effective amount of a composition of the invention.

The present invention further provides methods for reducing a subject's plasma fibrinogen level, comprising administering to a subject in need thereof an effective amount of a composition of the invention.

In some embodiments, the subject's fibrinogen level is greater than 300 mg/dl. In some embodiments, the subject's fibrinogen level is greater than 400 mg/dl.

The present invention further provides methods for reducing a fibrosis score or a nonalcoholic fatty liver disease activity score in a subject, comprising administering to a subject in need thereof an effective amount of a composition of the invention. The nonalcoholic fatty liver disease activity score (NAS or NAFLD score) is a composite score that measures changes in NAFLD during therapeutic trials. NAS is a composite score comprising three components that includes scores for steatosis, lobular inflammation and hepatocyte ballooning. NAS is defined as the unweighted sum of the scores for steatosis, lobular inflammation and hepatocyte ballooning. Steatosis grade can be quantified as the percentage of hepatocytes that contain fat droplets. The fibrosis stage of the liver is evaluated separately from NAS by histological evaluation of the intensity of sirius red staining of collagen in the pericentral region of liver lobules.

The present invention further provides methods for reducing elevated total cholesterol, low-density lipoprotein cholesterol (LDL-C), apolipoprotein B (Apo B), triglyceride or non-high-density lipoprotein cholesterol in a subject, comprising administering to a subject in need thereof an effective amount of a composition of the invention. The present invention further provides methods for increasing high-density lipoprotein cholesterol in a subject, comprising administering to a subject in need thereof an effective amount of a composition of the invention. In some embodiments, the subject has primary hyperlipidemia. In some embodiments, the primary hyperlipidemia is heterozygous familial. In some embodiments, the primary hyperlipidemia is non-familial. In some embodiments, the subject has mixed hyperlipidemia.

The present invention further provides methods for reducing elevated total cholesterol or elevated LDL-C in a subject, comprising administering to a subject in need thereof an effective amount of a composition of the invention. In some embodiments, the subject has homozygous familial hypercholesterolemia (HoFH). The methods of the invention may further comprise administering an additional therapeutic agent to a subject. The methods of the invention may further comprise administering two or more additional therapeutic agents to a subject.

In some embodiments, the additional pharmaceutically active agent can be a statin, lipid lowering agent, a PCSK9 (proprotein convertase subtilisin/kexin type 9) inhibitor, a cholesterol absorption inhibitor, a ACC (acetyl-CoA carboxylase) inhibitor, an ApoC-III (apolipoprotein C-III) inhibitor, an ATP citrate lyase inhibitor, a fish oil, a fibrate, a thyroid hormone beta receptor agonist, a farnesoid X receptor (FXR), a C—C chemokine receptor type 2 (CCR2)/C—C chemokine receptor type 5 (CCR5) inhibitor, a caspase protease inhibitor, an ASK-1 (apoptosis signal-regulating kinase 1) inhibitor, a galectin-3 protein, a NOX (nicotinamide adenine dinucleotide phosphate-oxidase) inhibitor, an ileal bile acid transporter, a peroxisome proliferator-activated receptor (PPAR) agonist, a PPAR dual agonist, a pan-PPAR agonist, a sodium-glucose co-transporter 2 (SGLT2) inhibitor, a dipeptidyl peptidase 4 (DPP4) inhibitor, a toll-like receptor antagonist, a human hormone FGF19, or a CETP (cholesterylester transfer protein) inhibitor. The additional therapeutic agent can be a lipid-lowering treatment or agent. The lipid-lowering treatment or agent can be ezetimibe.

The methods of the invention may further comprise administering a statin and ezetimibe.

The present invention further provides methods for treating or preventing heterozygous familial hypercholesterolemia (HeFH) in a subject, comprising administering to a subject in need thereof an effective amount of a composition of the invention. The present invention further provides methods for treating or preventing atherosclerotic cardiovascular disease (ASCVD) in a subject, comprising administering to a subject in need thereof an effective amount of a composition of the invention. In further embodiments, the atherosclerotic cardiovascular disease is a clinical atherosclerotic cardiovascular disease. In some embodiments, the subject is an adult. In some embodiments, the subject is on statin therapy. In some embodiments, the statin therapy is maximally tolerated statin therapy. In some embodiments, the methods further comprise administering a statin to the subject. In some embodiments, the subject has abnormally high LDL-C. In some embodiments, the maximally tolerated statin therapy is insufficient to lower the subject's LDL-C.

The present invention further provides methods for treating or preventing HoFH in a subject, comprising administering to a subject in need thereof an effective amount of a composition of the invention. In some embodiments, the subject is on one or more other low-density lipoprotein (LDL)—lowering therapies. In some embodiments, the methods further comprise administering an LDL-lowering therapy to the subject. Non-limiting examples of LDL-lowering therapies include statins, ezetimibe, bile acid binding resin, PCSK9 inhibitor, Juxtapid® (Lomitapide), Kynamro® (mipomersan sodium) and LDL apheresis. In some embodiments, the subject has abnormally high LDL-C. In some embodiments, the other LDL-lowering therapy is insufficient to lower the subject's LDL-C.

The present invention further provides methods for reducing risk of a cardiovascular event in a subject, comprising administering to a subject in need thereof an effective amount of a composition of the invention. In some embodiments, the subject has coronary heart disease (CHD). In some embodiments, the subject has a history of acute coronary syndrome (ACS). In some embodiments, the subject has been previously treated with a statin. In other embodiments, the subject has not been previously treated with a statin.

The present invention further provides methods for treating or preventing primary hypercholesterolemia, comprising administering to a subject in need thereof an effective amount of a composition of the invention. The primary hypercholesterolemia may be HeFH or non-familial hypercholesterolemia. In some embodiments, the present invention further provides methods for treating or preventing mixed hyperlipidemia in a subject, comprising administering to a subject in need thereof an effective amount of a composition of the invention. In some embodiments, the subject or the subject's symptoms are not effectively treated with a statin alone. In some embodiments, the subject was treated with a statin and/or ezetimibe previously. In some embodiments, the methods further comprise administering a one or both of a statin and ezetimibe to the subject.

The present invention further provides methods for treating or preventing HoFH in a subject, comprising administering to a subject in need thereof an effective amount of a composition of the invention. In some embodiments, the method further comprises administering an adjunctive treatment. The adjunctive treatment can be one or more of a statin, ezetimibe, bile acid binding resin, PCSK9 inhibitor, Juxtapid® (Lomitapide), Kynamro® (mipomersan sodium) and LDL apheresis.

The present invention further provides methods for reducing risk of myocardial infarction, stroke, revascularization procedures or angina in a subject, comprising administering to a subject in need thereof an effective amount of a composition of the invention. In some embodiments, the subject does not have coronary heart disease (CHD). In some embodiments, the subject has one or more risk factors for CHD.

The present invention further provides methods for reducing a subject's risk of myocardial infarction or stroke, comprising administering to a subject in need thereof an effective amount of a composition of the invention. In some embodiments, the subject has type 2 diabetes. In some embodiments, the subject has type 2 diabetes and does not have CHD. In some embodiments, the subject has one or more risk factors for CHD.

The present invention further provides methods for reducing a subject's risk of non-fatal myocardial infarction, fatal stroke or non-fatal stroke, need for a revascularization procedure, hospitalization for congestive heart failure (CHF) or angina, comprising administering to a subject in need thereof an effective amount of a composition of the invention. In some embodiments, the subject has CHD.

The present invention further provides methods for reducing elevated total cholesterol, LDL-C, apolipoprotein B or triglyceride in a subject, comprising administering to a subject in need thereof an effective amount of a composition of the invention. The present invention further provides methods for increasing high-density lipoprotein cholesterol in a subject, comprising administering to a subject in need thereof an effective amount of a composition of the invention. In some embodiments, the subject is an adult. In some embodiments, the subject has primary hyperlipidemia. Primary hyperlipidemia can be heterozygous familial or non-familial. In some embodiments, the subject has mixed dyslipidemia.

The present invention further provides methods for reducing elevated triglyceride in a subject, comprising administering to a subject in need thereof an effective amount of a composition of the invention. In some embodiments, the subject has hypertriglyceridemia. In some embodiments, the subject has primary dysbetalipoproteinemia. In yet some other embodiments, the subject has hypoalphalipoproteinemia.

The present invention further provides methods for reducing total cholesterol or LDL-C in a subject, comprising administering to a subject in need thereof an effective amount of a composition of the invention. In some embodiments, the subject has HoFH.

The present invention further provides methods for reducing elevated total cholesterol, LDL-C or apolipoprotein B in a subject, comprising administering to a subject in need thereof an effective amount of a composition of the invention. In some embodiments, the subject is a human male or a human female (e.g., postmenarcheal female) who is 10-17 years of age. In some embodiments, the subject has HeFH. In some embodiments, the subject's diet is insufficient to reduce the patient's elevated total cholesterol, LDL-C or Apo B.

The present invention further provides methods for reducing a subject's risk of mortality, CHD death, non-fatal myocardial infarction, stroke or need for a revascularization procedure, comprising administering to a subject in need thereof an effective amount of a composition of the invention. In some embodiments, the subject is at high risk of coronary events.

The present invention further provides methods for reducing elevated total cholesterol, LDL-C, apolipoprotein B or triglyceride in a subject, comprising administering to a subject in need thereof an effective amount of a composition of the invention. The present invention further provides methods for increasing high-density lipoprotein cholesterol in a subject, comprising administering to a subject in need thereof an effective amount of a composition of the invention. In some embodiments, the subject has primary hyperlipidemia. In some embodiments, the primary hyperlipidemia is heterozygous familial hyperlipidemia. In some embodiments, the primary hyperlipidemia is non-familial hyperlipidemia. In some embodiments, the subject has mixed dyslipidemia.

The present invention further provides methods for reducing elevated triglyceride in a subject, comprising administering to a subject in need thereof an effective amount of a composition of the invention. In some embodiments, the subject has hypertriglyceridemia. The present invention further provides methods for reducing triglyceride or very-low-density lipoprotein cholesterol (VLDL-C) in a subject, comprising administering to a subject in need thereof an effective amount of a composition of the invention. In some embodiments, the subject has primary dysbetalipoproteinemia.

The present invention further provides methods for reducing elevated total cholesterol or LDL-C in a subject, comprising administering to a subject in need thereof an effective amount of a composition of the invention. In some embodiments, the subject is an adult. In some embodiments, the subject has HoFH.

The present invention further provides methods for treating or preventing hypertriglyceridemia in a subject, comprising administering to a subject in need thereof an effective amount of a composition of the invention. In some embodiments, the method further comprises adjusting the subject's diet.

The present invention further provides methods for treating or preventing primary dysbetalipoproteinemia in a subject, comprising administering to a subject in need thereof an effective amount of a composition of the invention. In some embodiments, the primary dysbetalipoproteinemia is Type III hyperlipoproteinemia. In some embodiments, the method further comprises adjusting the subject's diet.

The present invention further provides methods for reducing total cholesterol, LDL-C or apolipoprotein B in a subject, comprising administering to a subject in need thereof an effective amount of a composition of the invention. In some embodiments, the subject has HoFH.

The present invention further provides methods for reducing elevated LDL-C, total cholesterol, apolipoprotein B or triglyceride in a subject, comprising administering to a subject in need thereof an effective amount of a composition of the invention. The present invention further provides methods for increasing high-density lipoprotein cholesterol in a subject, comprising administering to a subject in need thereof an effective amount of a composition of the invention. In some embodiments, the subject is an adult. In some embodiments, the subject has primary hypercholesterolemia. In some embodiments, the subject has mixed dyslipidemia.

The present invention further provides methods for treating or preventing severe hypertriglyceridemia in a subject, comprising administering to a subject in need thereof an effective amount of a composition of the invention. In some embodiments, the subject is an adult.

The present invention further provides methods for reducing the rate of myocardial infarction or stroke in a subject, comprising administering to a subject in need thereof an effective amount of a composition of the invention. In some embodiments, the subject has acute coronary syndrome (ACS). In some embodiments, the subject has non-ST-segment elevation ACS (unstable angina (UA)/non-ST-elevation myocardial infarction (NSTEMI)). In some embodiments, the subject has ST-elevation myocardial infarction (STEMI). In electrocardiography, the ST segment connects the QRS complex and the T wave. In some embodiments, the subject has had a previous myocardial infarction, previous stroke or established peripheral arterial disease. In some embodiments, the subject has had a recent myocardial infarction or recent stroke.

The present invention further provides methods for reducing total cholesterol, LDL-C or Apo B in a subject, comprising administering to a subject in need thereof an effective amount of a composition of the invention. In some embodiments, the subject has primary hypercholesterolemia. Primary hypercholesterolemia can be heterozygous familial or non-familial. In some embodiments, the method further comprises administering an HMG-CoA reductase inhibitor to the subject.

The present invention further provides methods for reducing total cholesterol or LDL-C levels in a subject, comprising administering to a subject in need thereof an effective amount of a composition of the invention. In some embodiments, the subject has HoFH. In some embodiments, the method further comprises administering an additional lipid-lowering treatment to the subject. In some embodiments, the additional lipid-lowering treatment may be a statin (e.g., atorvastatin or simvastatin) or LDL apheresis.

The present invention further provides methods for reducing elevated sitosterol or campesterol levels in a subject, comprising administering to a subject in need thereof an effective amount of a composition of the invention. In some embodiments, the subject has homozygous familial sitosterolemia.

The present invention further provides methods for treating or preventing Type IV or Type V hyperlipidemia in a subject, comprising administering to a subject in need thereof an effective amount of a composition of the invention. In some embodiments, the subject has a risk of pancreatitis. In some embodiments, the subject does not respond adequately to a dietary change to control elevations of serum triglyceride levels. In some embodiments, the subject has an abnormally high serum triglyceride level. In some embodiments, the subject has a serum triglyceride level of over 2000 mg/dl and optionally has an elevation of VLDL-cholesterol as well as fasting chylomicrons. In some embodiments, the subject has a triglyceride of from 1000 to 2000 mg/dl and optionally has a history of pancreatitis or of recurrent abdominal pain typical of pancreatitis.

The present invention further provides methods for reducing risk of developing coronary heart disease a subject, comprising administering to a subject in need thereof an effective amount of a composition of the invention. In some embodiments, the subject has Type IIb hyperlipidemia. In some embodiments, the subject does not have history of or symptoms of existing coronary heart disease. In some embodiments, the subject has had weight loss, dietary therapy, exercise, or was administered another pharmacologic agent (e.g., a bile acid sequestrant or nicotinic acid) that was ineffective to treat the subject's hyperlipidemia. In some embodiments, the subject has one or more of an abnormally low HDL-cholesterol level, an abnormally high LDL-cholesterol level and an abnormally high triglyceride level.

In some embodiments, the methods of the invention further comprise administering an effective amount of an additional pharmaceutically active agent. In some embodiments, the methods of the invention further comprise administering an effective amount of two or more additional pharmaceutically active agents.

In some embodiments, the additional pharmaceutically active agent is a statin. In some embodiments, statin is atorvastatin, simvastatin, pravastatin, rosuvastatin, fluvastatin, lovastatin, pitavastatin, mevastatin, dalvastatin, dihydrocompactin, or cerivastatin, or a pharmaceutically acceptable salt thereof. In some embodiments, the statin is atorvastatin calcium.

In some embodiments, the additional pharmaceutically active agent is another statin. In some embodiments, the additional pharmaceutically active agent is an HMG-CoA (3-hydroxy-3-methyl-glutaryl-coenzyme A) reductase inhibitor.

In some embodiments, the additional pharmaceutically active agent is a lipid-modifying agent, lipid lowering agent, anti-fibrolytic agent, or an anti-inflammatory agent. In some embodiments, the additional pharmaceutically active agent is a cholesterol-lowering agent. In other embodiments, the additional pharmaceutically active agent is a cholesterol absorption inhibitor. In other embodiments, the cholesterol absorption inhibitor is ezetimibe.

In some embodiments, the additional pharmaceutically active agent is a a PCSK9 (proprotein convertase subtilisin/kexin type 9) inhibitor, a cholesterol absorption inhibitor, an ACC (acetyl-CoA carboxylase) inhibitor, an ApoC-III (apolipoprotein C-III) inhibitor, an ApoB (apolipoprotein B) synthesis inhibitor, an ACL (adenosine triphosphate citrate lyase) inhibitor, a microsomal transfer protein inhibitor, a fenofibric acid, a fish oil, a fibrate, a thyroid hormone beta receptor agonist, a farnesoid X receptor (FXR), a CCR2/CCR5 inhibitor, a caspase protease inhibitor, an ASK-1 inhibitor, a galectin-3 protein, a NOX inhibitor, an ileal bile acid transporter, a PPAR agonist, a PPAR dual agonist, a pan-PPAR agonist, a sodium-glucose co-transporter 2 (SGLT2) inhibitor, a dipeptidyl peptidase 4 (DPP4) inhibitor, a toll-like receptor antagonist, a human hormone FGF19, or a CETP (cholesterylester transfer protein) inhibitor. In other embodiments, the additional lipid lowering agent is PCKS9 inhibitor. In some embodiments, the additional lipid lowering agent is bempedoic acid, nicotinic acid, gemfibrozil, niacin, a bile-acid resin, a fabric acid derivative, or a cholesterol absorption inhibitor. In some embodiments, the additional lipid lowering agent is bempedoic acid, nicotinic acid, or gemfibrozil. In some embodiments the lipid-reducing agent is gemfibrozil. In some embodiments, the one or more pharmaceutically active agent is bempedoic acid.

Examples of fish oil include, but are not limited to, salmon oil, sardine oil, cod liver oil, tuna oil, herring oil, menhaden oil, mackerel oil, refined fish oils, and mixtures thereof. Fish oils contain omega-3 fatty acids eicosapentaenoic acid and docosahexaenoic acid. In some embodiments, the fish oil is prescription fish oil.

In some embodiments, the CETP inhibitor is dalcetrapib (CAS 211513-37-0), torcetrapib (CAS 262352-17-0), anacetrapib (CAS 875446-37-0), evacetrapib (CAS 1186486-62-3), BAY 60-5521 (CAS 893409-49-9), obicetrapib (866399-87-3), ATH-03 (Affris), DRL-17822 (Dr. Reddy's), DLBS-1449 (Dexa Medica), S-[2-[1-(2-ethylbutyl)cyclohexylcarbonylamino]phenyl]-2-methylthiopropionate, 1-(2-ethyl-butyl)-cyclohexanecarboxylic acid (2-mercapto-phenyl)-amide or bis[2-[1-(2-ethylbutyl)cyclohexylcarbonylamino]phenyl]disulfide, or pharmaceutically acceptable salt thereof.

In some embodiments, the additional pharmaceutically active agent is an antibody to CETP. In some embodiments, the antibody to CETP is a monoclonal antibody. In other embodiments, the antibody to CETP is a monoclonal antibody (Mab, TP1) to CETP.

In some embodiments, the additional pharmaceutically active agent induces antibodies against CETP. In some embodiments, the additional pharmaceutically active agent which induces antibodies against CETP is a vaccine. In some embodiments, the vaccine is TT/CETP (Rittershaus, C. W. et al., Arteriosclerosis, Thrombosis, and Vascular Biology. 2000; 20:2106-2112). In other embodiments, the additional pharmaceutically active agent which induces antibodies against CETP is CETi-1 (Celldex Therapeutics).

In some embodiments, the additional pharmaceutically active agent immunizes a subject with CETP or CETP protein fragment.

In some embodiments, the additional pharmaceutically active agent reduces CETP by inhibition with an siRNA to CETP mRNA.

In some embodiments, the additional pharmaceutically active agent targets CETP transcription by administration of DNAi to the CETP gene. In other embodiments, the additional pharmaceutically active agent targets CETP transcription by administration of DNAi in an appropriate deliver vehicle such as a SMARTICLE™.

In some embodiments, the additional pharmaceutically active agent is an anti-coagulation agent or a lipid-regulating agent. In some embodiments the anti-coagulation agent is aspirin, dabigatran, rivaroxaban, apixaban clopidogrel, clopNPT (conjugate of clopidogrel with 3-nitropyridine-2-thiol), thienopyridine, warfarin (Coumadin) acenocoumarol, phenprocoumon, atromentin, phenindione, edoxaban betrixaban, letaxaban eribaxaban hirudin, lepirudin, bivalirudin, argatroban, dabigatran, ximelagatran, batroxobin, hementin, a heparin or vitamin E.

In some embodiments, the additional pharmaceutically active agent is simtuzumab (CAS 1318075-13-6), selonsertib (CAS 1448428-04-3), GS-9674 (Gilead Sciences), GS-0976 (Gliead Sciences), obeticholic acid (CAS 459789-99-2), or cenicriviroc (CAS 497223-25-3), or a pharmaceutically acceptable salt thereof.

In some embodiments, the additional pharmaceutically active agent is an anti-inflammatory agent, an anti-hypertensive agent, an anti-diabetic agent, an anti-obesity, an anti-fibrotic or an anti-coagulation agent.

In some embodiments, the additional pharmaceutically active agent disclosed herein can be a pharmaceutically acceptable salt thereof. The pharmaceutically acceptable salt can be an acid addition salt where the pharmaceutically active agent is basic, e.g., includes a basic nitrogen atom, and can be a cationic salt. In some embodiments, examples of inorganic or organic acids suitable to form an acid addition salt, include but are not limited to, hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. The pharmaceutically acceptable salt can be a base addition salt where the pharmaceutically active agent is acidic.

In some embodiments, the methods of the invention do not induce hepatotoxicity or a musculoskeletal disorder.

In some embodiments, any one of the methods as disclosed herein can be useful for patients on statin therapy. In some embodiments, the statin is atorvastatin, simvastatin, pravastatin, rosuvastatin, fluvastatin, lovastatin, pitavastatin, mevastatin, dalvastatin, dihydrocompactin, or cerivastatin, or a pharmaceutically acceptable salt thereof. In some embodiments, the statin is atorvastatin calcium.

In some embodiments, a pharmaceutical composition of the invention is administered to a subject in need thereof once per day.

### EXAMPLES

### Example 1: Atorvastatin-Gemcabene Tablet Formulations

Table 2 and Table 3 show the compositions of formulations containing polyvinylpyrrolidone (PVP) and hydroxypropyl cellulose (HPC), respectively.

TABLE 2
Composition of Formulations Containing PVP as a Binder

| Ingredients | Example 1A | Example 1B | Example 1C |
|---|---|---|---|
| Base Granulation | wt % of the composition | | |
| Gemcabene Calcium | 58.1 | 58.1 | 19.3 |
| Salt | | | |
| Atorvastatin Calcium | 0.588 | 2.4 | 4.7 |
| CaCO₃ | 0-1.77 | 0-7.1 | 0-14.1 |
| Microcrystalline | 15-13.8 | 13.7-6.7 | 50.1-36.0 |
| Cellulose (PH 101) | | | |
| PVP K-30^{a} | 6.7 | 6.7 | 6.7 |
| Croscarmellose Sodium | 3.0 | 3.0 | 3.0 |
| Final Blend | wt % | | |
| Microcrystalline | 12.3 | 12.3 | 12.3 |
| Cellulose (PH 102) | | | |
| Croscarmellose Sodium | 3.0 | 3.0 | 3.0 |
| Magnesium stearate | 0.8 | 0.8 | 0.8 |
| (non-bovine) | | | |
| Gemcabene/Atorvastatin | 225/2.5 | 225/10 | 75/20 |
| (mg/mg) | | | |

^{a}Binder added partially as powder (3%) and partially as solution (3.7%)

TABLE 3
Composition of Formulations Containing Hydroxypropyl
Cellulose as a Binder

| Ingredients | Example 1D | Example 1E | Example 1F |
|---|---|---|---|
| Base Granulation | wt % of the composition | | |
| Gemcabene Calcium | 58.1 | 58.1 | 19.3 |
| Salt | | | |
| Atorvastatin Calcium | 0.588 | 2.4 | 4.7 |
| CaCO₃ | 0-1.76 | 0-7.1 | 0-14.1 |
| Microcrystalline | 4.2-2.5 | 2.5-0 | 38.9-24.8 |
| Cellulose (PH 101) | | | |
| Starch, Pregelatinized, | 10.0 | 10.0 | 10.0 |
| 1500 Corn | | | |
| Hydroxypropyl | 8.0 | 8.0 | 8.0 |
| Cellulose-EXF^{a} | | | |
| Croscarmellose Sodium | 3.0 | 3.0 | 3.0 |
| Final Blend | wt % of the composition | | |
| Microcrystalline | 12.2 | 12.2-7.7 | 12.2 |
| Cellulose (PH 102) | | | |
| Croscarmellose Sodium | 3.0 | 3.0 | 3.0 |
| Magnesium stearate | 0.8 | 0.8 | 0.8 |
| (non-bovine) | | | |
| Gemcabene/Atorvastatin | 225/2.5 | 225/10 | 75/20 |
| (mg/mg) | | | |

^{a}Binder added partially as powder (4%) and partially as solution (4%)

The following major equipment were used in the manufacture of the tablets for this study:

Tekmar RW20 DZM mixer;
Masterflex pump, model 7523-10;
Bohle Mini-Granulator equipped with 4 L bowl;

### Hotpack Benchtop Oven (Model 213023-25);

Computrac Max 2000 moisture analyzer;
Quadro Comil 193AS (equipped with 0.045 inch screen, impeller 1601, and spacer 175);
Patterson-Kelly Blendmaster twin-shell blender (4 qt); and
Korsch EKO (SN K0000060) equipped with 14/32 inch round concave (plain-faced) tooling.

Binder solution (15% w/w) was prepared by slow addition of either HPC or PVP to the required weight of water while mixing using a Tekmar mixer. The mixing was continued for at least 2 hours until all the binder was in solution. The solution was then allowed to stand for few hours (typically overnight) before use to ensure that there were no air-bubbles.

Base granulations were prepared at 300 g scale using the Bohle High-Shear minigranulator equipped with a 4 L bowl. All the ingredients of the base granulation including a portion of binder that is added as powder (Table 2 and Table 3) were taken in a plastic bag and mixed. The mixture was charged into the 4 L bowl and mixed further using the impeller at 300 rpm, typically for 1 to 2 minutes. Binder solution was then added at a constant flow rate (9.3 g/min for PVP and 20 g/min for HPC) while mixing using an impeller speed of 300 rpm and a chopper speed of 1500 rpm. After complete addition of the binder solution, water was added without changing the pump setting or the impeller and chopper speeds. The quantity of total water added for granulation varied for each formulation, with a higher percentage of water required to aid formation of granules as the microcrystalline cellulose (PH 101) content was increased. The granulation was mixed further at the same impeller/chopper speed until the granulation end-point was reached (based on visual appearance). Typical total granulation times were about 9 to 11 or 16 to 19 minutes for formulations containing HPC and PVP, respectively. The granulations were tray-dried in a Hotpack benchtop oven at 50° C. until about 2% LOD (loss on drying) was reached.

The base granulations were milled using a Quadro Comil Model 193AS equipped with a 0.045 inch screen, impeller 1601, and spacer 175, at 2220 rpm (Setting 6) or 2920 rpm (Setting 8). The milled base granulation was blended with microcrystalline cellulose (PH 102) and croscarmellose sodium for 5 minutes using a 4 qt twin-shell blender. Magnesium stearate was added to a small portion of this blend, and the mixture was passed through 30-mesh screen. After addition of this screened material to the rest of the batch in the blender, blending was continued for another 3 minutes to obtain the final blend.

The final blend was compressed into compacts using a single station stationary press, Korsch EKO, equipped with 14/32 inch round concave (plain-faced) tooling. The target weight and hardness for the tablets were 465 mg and 15-25 kP, respectively.

The tablets of Examples 1A-1F were subjected to accelerated stability testing, wherein 15 tablets of each example tablet were stored in 60 cc high-density polyethylene bottles. Test bottles of the tablets included unsealed bottles (“Open”) and bottles sealed by foil induction (“Closed”). A first test group of Open bottles was subjected to 40° C. and 75% relative humidity; a second test group of Closed bottles was subjected to the same conditions; and a third test group of Closed bottles was subjected to 60° C. and ambient humidity. Tablets were analyzed for oxidized atorvastatin and lactone formation using HPLC and UV spectroscopy analytical methods. Tablets from the second test group were tested for initial stability. Selected tablets from each of the bottles of the test groups were analyzed at 1 month intervals. The tablets of Examples 1A-1C that were formulated with PVP demonstrated unacceptable levels of oxidation at the 1-month time point, so testing of these tablets ceased. The remaining tablets of examples 1D-1F continued to be tested for a period of more than 7 months.

Shelf life for each of the tablets of examples 1D-1F was determined using the stability data of this test to estimate pseudo zeroth order rate constants at both 40° C. and 60° C. The shelf lives for the tablets of examples 1E and 1F at 25° C. were estimated to be about 4 years while the shelf life for the tablet of example 1D was estimated to be less than 2 years.

Moreover, it was observed that the addition of CaCO₃ in the tablet formulations suppressed lactone formation. For a given time, storage condition, and mass of gemcabene per mass atorvastatin, the lactone formation was higher in the 0×CaCO₃ versus the 3×CaCO₃. And surprisingly, atorvastatin lactone formation rates were found to be greatest in the tablets of example 1D and least in the tablets of example 1F, which indicates that little or no correlation exists between the rate of lactone formation and the loading of gemcabene in the tablet formulation.

### Example 2: Formulation Suitability Study for Combination Atorvastatin and Gemcabene Tablets

This study was an open label, single dose, randomized, 6-sequence, 6-period, 6-treatment crossover study conducted in healthy volunteers. Eighteen subjects entered the study and were to receive each of the following treatments:

Reference: Atorvastatin 40 mg tablet alone (LIPITOR® (atorvastatin calcium))
Tests: Gemcabene+Atorvastatin tablet Formulations containing 450 mg gemcabene and 40 mg atorvastatin in Experiments 2A-2D, and containing 300 mg gemcabene and 10 mg atorvastatin in Experiment 2E as described in Tables 4 and 5.

TABLE 4
Gemcabene/Atorvastatin (G/A) Formulations 2A-2C
(% w/w = % of the total tablet weight)

| | Example 2A | | Example 2B | | Example 2C | |
|---|---|---|---|---|---|---|
| | (450/40 mg G/A) | | (450/40 mg G/A) | | (450/40 mg G/A) | |
| | % w/w | mg/Tablet | % w/w | mg/Tablet | % w/w | mg/Tablet |
| Internal Components | | | | | | |
| Gemcabene Calcium salt | 56.91 | 540.61 | 50.18 | 540.61 | 54.06 | 540.61 |
| Atorvastatin Ca | 4.61 | 43.78 | 4.06 | 43.78 | 4.38 | 43.78 |
| Calcium Carbonate | 0.00 | 0.00 | 12.19 | 131.32 | 0.00 | 0.00 |
| Microcrystalline | 4.00 | 38.00 | 3.50 | 37.71 | 3.80 | 38.01 |
| Cellulose, NF | | | | | | |
| (PH 101) | | | | | | |
| Starch 1500 | 0.00 | 0.00 | 0.00 | 0.00 | 0.00 | 0.00 |
| Croscarmellose Sodium | 3.00 | 28.50 | 3.00 | 32.32 | 2.85 | 28.50 |
| Hydroxypropyl | 5.00 | 47.50 | 5.30 | 57.10 | 4.75 | 47.50 |
| Cellulose EXF | | | | | | |
| External Ingredients | | | | | | |
| Microcrystalline | 22.49 | 213.62 | 17.77 | 191.49 | 20.01 | 200.11 |
| Cellulose, NF (PH 102) | | | | | | |
| Mannitol | 0.00 | 0.00 | 0.00 | 0.00 | 6.00 | 60.00 |
| Croscarmellose Sodium | 3.00 | 28.50 | 3.00 | 32.32 | 3.15 | 31.50 |
| Magnesium Stearate | 1.00 | 9.50 | 1.00 | 10.77 | 1.00 | 10.00 |
| (Nonbovine) | | | | | | |
| To make core Tablets | 100.00 | 950.00 | 100.00 | 1077.42 | 100.00 | 1000.01 |

| | Without CaCO₃ | With CaCO₃ | Fast, Without CaCO₃ |
|---|---|---|---|
| % Atorvastatin | 22 | 22 | 43 |
| Dissolution at 10 Min | | | |
| % Atorvastatin | 49 | 59 | 66 |
| Dissolution at 20 Min | | | |
| Hardness (kP) | 30 kP | 30 kP | 31 kP |
| Disintegration Time (Min) | 17.2 | 13.8 | 10.4 |

TABLE 5
Gemcabene/Atorvastatin (G/A) Formulations 2D and 2E
(% w/w = % of the total tablet weight)

| | Example 2D | | Example 2E | |
|---|---|---|---|---|
| | (450/40 mg G/A) | | (300/10 mg G/A) | |
| | % w/w | mg/Tablet | % w/w | mg/Tablet |
| Internal | | | | |
| Ingredients | | | | |
| Gemcabene | 50.18 | 540.61 | 56.91 | 360.40 |
| Calcium salt | | | | |
| Atorvastatin Ca | 4.06 | 43.78 | 1.73 | 10.94 |
| Calcium | 12.19 | 131.32 | 0.00 | 0.00 |
| Carbonate | | | | |
| Microcrystalline | 3.50 | 37.71 | 4.00 | 25.33 |
| Cellulose, NF | | | | |
| (PH 101) | | | | |
| Starch 1500 | 6.50 | 70.03 | 0.00 | 0.00 |
| Croscarmellose | 3.00 | 32.32 | 3.00 | 19.00 |
| Sodium | | | | |
| Hydroxypropyl | 5.30 | 57.10 | 5.00 | 31.67 |
| Cellulose EXF | | | | |
| External | | | | |
| Ingredients | | | | |
| Microcrystalline | 11.27 | 121.46 | 25.37 | 160.65 |
| Cellulose, NF | | | | |
| (PH 102) | | | | |
| Mannitol | 0.00 | 0.00 | 0.00 | 0.00 |
| Croscarmellose | 3.00 | 32.32 | 3.00 | 19.00 |
| Sodium | | | | |
| Magnesium | 1.00 | 10.77 | 1.00 | 6.33 |
| Stearate | | | | |
| (Nonbovine) | | | | |
| To make core | 100.00 | 950.00 | 100.00 | 1077.42 |
| Tablets | | | | |

| | Slow, With CaCO₃ | (300/10) No CaCO₃ |
|---|---|---|
| % Atorvastatin | 18 | Disintegration Faster |
| Dissolution at | | Than 450/40 |
| 10 Min | | |
| % Atorvastatin | 41 | |
| Dissolution at | | |
| 20 Min | | |
| Hardness (kP) | 29 kP | 17 kP |
| Disintegration | 29.8 | 12.3 |
| Time (Min) | | |

### Diagnosis and Main Criteria for Inclusion:

Healthy subjects of any race and either gender; age 18 to 65 (inclusive), with a body weight of 45 kg or greater and a body mass index (BMI)≤35 kg/m² (weight [kg]/height [meters]²); females required to be of non-reproductive potential (postmenopausal ≥1 year, hysterectomy, or tubal ligation).

### Exclusion Criteria:

Use of any medication not considered acceptable by the clinical investigators during the 14-day period before the start of the study (Day 1). Hormone replacement therapy is acceptable;

Donation of a unit of blood or participation in a study of investigational or marketed drugs during the 30-day period before the start of the study (Day 1);

If female, of childbearing potential or lactating;

Use of St. John's wort during the 7-day period before the start of the study (Day 1);

Consumption of grapefruit juice or food products containing grapefruit during the 7-day period before the start of study (Day 1);

History of significant adverse reaction to any lipid-lowering agent; or

Significant urine concentration of any drug that could interfere with the study.

### Duration of Treatment:

Single dose Gemcabene+atorvastatin tablet formulations 1-5 with a minimum 2-week washout period between treatments.

On Days 1, 15, 29, 50, 64, and 78 subjects were randomized to receive an oral, single dose of one of the five gemcabene+atorvastatin formulations or atorvastatin tablets. Each single dose is administered with 40 mL (8 oz.) of water.

### Results:

Eighteen subjects (12 male, 6 female) entered the study, and were withdrawn from the study on Day 64 due to the early termination of the study. Subjects had a mean (range) age of 52.6 (28-64) years, a mean (range) weight of 88.7 (55.4-111) kg, and a mean (range) BMI of 29.0 (21.6-34.1) kg/m².

Formulation 2A: 8 of 12 subjects reported adverse events. There were no severe adverse events. The most frequently occurring adverse event was infection (3 subjects). All other adverse events were single occurrences. Three subjects reported adverse events that were considered treatment associated: Anorexia, dizziness, and dry mouth (1 subject each). Ten adverse events were considered mild in intensity, and 1 adverse event was considered moderate in intensity.

Formulation 2B: 7 of 12 subjects reported adverse events. The most frequently occurring adverse events were headache and somnolence (2 subjects). All other adverse events were single occurrences. 5 subjects reported adverse events that were considered treatment associated: Somnolence (2 subjects) and diarrhea, asthenia, and dyspepsia (1 subject each). Eight adverse events were considered mild in intensity, 1 adverse event was considered moderate in intensity, and 1 adverse event (headache) was considered severe in intensity. The severe adverse event was not considered treatment associated.

Formulation 2C: 9 of 12 subjects reported adverse events. There were no severe adverse events. The most frequently occurring adverse events were infection (3 subjects) and headache and somnolence (2 subjects). All other adverse events were single occurrences. Four subjects reported adverse events that were considered treatment associated: Headache, asthenia, somnolence, and tachycardia, (1 subject each). Eight adverse events were considered mild in intensity and 4 adverse events were considered moderate in intensity.

Formulation 2D: 8 of 12 subjects reported adverse events. There were no severe adverse events. The most frequently occurring adverse events were headache (3 subjects) and somnolence (2 subjects). All other adverse events were single occurrences. 4 subjects reported adverse events that were considered treatment associated: Somnolence (2 subjects) and headache and dyspepsia (1 subject each). Seven adverse events were considered mild in intensity, and 5 adverse events were considered moderate in intensity.

Formulation 2E: 6 of 12 subjects reported adverse events. There were no severe adverse events. The most frequently occurring adverse event was headache (2 subjects). All other adverse events were single occurrences. Three subjects reported adverse events that were considered treatment associated: Headache (2 subjects) and diarrhea (1 subject). 8 adverse events were considered mild in intensity, and 3 adverse events were considered moderate in intensity.

Atorvastatin 40 mg: 8 of 11 subjects reported adverse events. There were no severe adverse events. The most frequently occurring adverse events were headache (3 subjects) and dizziness and pain (2 subjects). All other adverse events were single occurrences. 2 subjects reported adverse events that were considered treatment associated: Headache and diarrhea (1 subject each). Nine adverse events were considered mild in intensity and 3 adverse events were considered moderate in intensity.

### Conclusions:

Single doses of combination gemcabene/atorvastatin Formulations 4A-4E are safe and well-tolerated by healthy volunteers.

### Example 3: Atorvastatin Calcium Capsule Formulation

Atorvastatin calcium capsules (20 mg) were prepared as neat drug substance in capsules as batch 121-16001. Atorvastatin Calcium Trihydrate drug substance was manually filled into size 0 white opaque gelatin capsules shells, by weight, to give a dosage strength of 20 mg Atorvastatin (see Table 6 below).

TABLE 6
Formulation Composition and Batch Size of the Atorvastatin Capsules

| | | | Amount/ | |
|---|---|---|---|---|
| Item | | Concentration | Capsule | Amount/ |
| No. | Ingredient | % w/w | (mg) | Batch (g) |
| 1 | Atorvastatin Calcium | 18.85 | 21.79 | 1.3074 |
| | Salt, Trihydrate | | | |
| 2 | Size 0 White Opaque | 81.15 | 93.8 | 5.628 |
| | Gelatin Capsules | | | |
| | Total | 100.0 | 115.29 | 6.9354 |

Dissolution Profile:

Dissolution testing was performed using USP Apparatus 2 (paddles) at 75 RPM in 900 mL deionized water with samples collected at 10, 20, 30, 45, and 60 minutes (FIG. 1C).

### Example 4: Gemcabene Calcium Capsule Formulation

Gemcabene calcium capsules (150 mg) were prepared as batch 121-16002. The capsule fill was prepared as a high-shear wet granulation using a lab-scale high-shear granulator (Vector/Freund GMX-LabMini). The ingredients, items 1-5, were added to the granulator bowl and granulated by the addition of 20% w/w (relative to the granulator charge) purified water. Resulting granulation was sieved through a #10 mesh sieve and subsequently dried in a lab-scale fluid-bed dryer (Vector/Freund MFL-01) to a final loss on drying (LOD) value of <3% as determined by a moisture balance. The dried granulation was passed through a #20 mesh sieve and blended in a diffusional blender (PK V-Blender) with magnesium stearate (Ingredient Item 6). The final blended material was encapsulated in size 00 white opaque capsule shells, using a Profil (Torpac) hand encapsulation tray, at 360 mg per capsule to give a final Gemcabene (free di acid) potency of 150 mg. The quantitative composition of gemcabene capsules, 150 mg and Batch Size are provided in Table 7 below.

TABLE 7
Formulation Composition and Batch Size of the Gemcabene Capsules

| Item | | Concentration | Mg/ | Amount/ |
|---|---|---|---|---|
| No. | Ingredient | % w/w | Capsule | Batch (g) |
| 1 | Gemcabene Calcium* | 49.02 | 176.47 | 114.706 |
| 2 | Lactose Monohydrate, | 25.48 | 91.73 | 59.624 |
| | NF (Granulac 70) | | | |
| 3 | Hydroxypropylcellulose, | 2.0 | 7.2 | 4.68 |
| | NF (Klucel EXF) | | | |
| 4 | Microcrystalline Cellulose, | 20.0 | 72.0 | 46.8 |
| | NF (Avicel PH102) | | | |
| 5 | Croscarmellose | 3.0 | 10.8 | 7.02 |
| | Sodium, NF | | | |
| 6 | Water, Purified | — | — | 47.3 |
| 7 | Magnesium stearate | 0.5 | 1.8 | 1.17 |

| | | | |
|---|---|---|---|
| Total | 100.0 | 360.0 | 234.0 |

*Gemcabene calcium salt is equivalent to 150 mg of gemcabene (di acid)

Dissolution Profile:

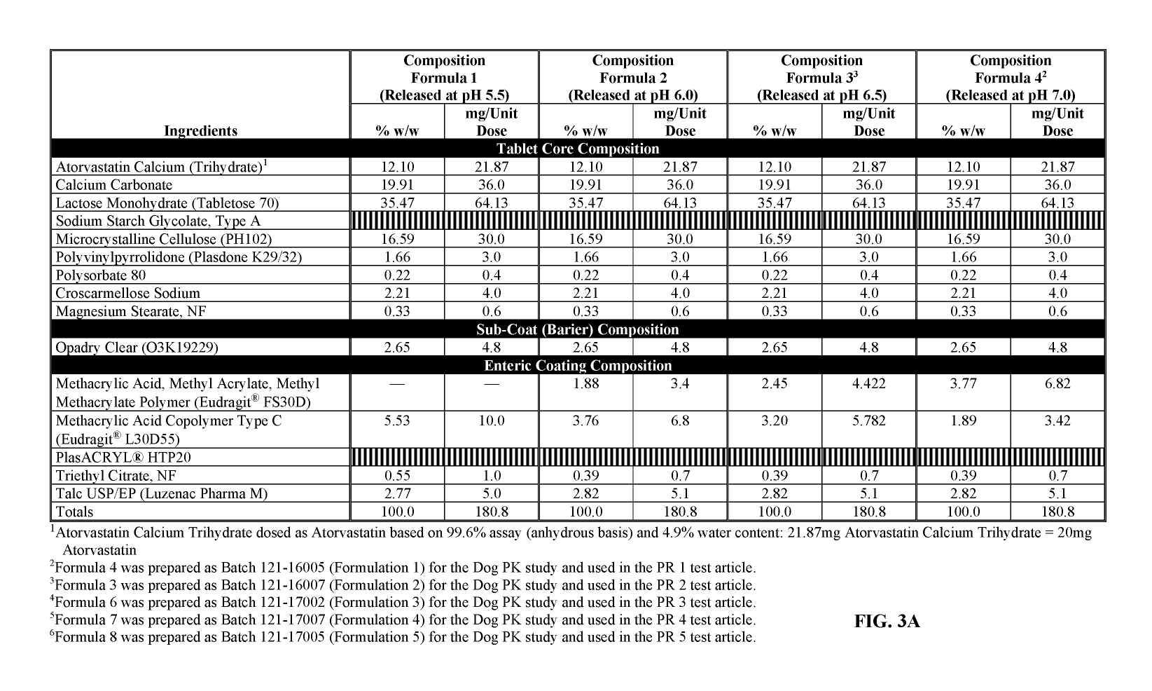

Dissolution testing was performed using USP Apparatus 2 (paddles) at 75 RPM in 900 mL deionized water with samples collected at 10, 20, 30, 45, and 60 minutes (FIG. 2).

### Example 5: Atorvastatin Calcium Formulation 1—Used in PR 1 Test Article in Dog Pharmacokinetic Study

Compositions of various formulations of atorvastatin having released at various pH values are presented in Tables 23 and 24. Tables 23 and 24 are tables providing compositions of atorvastatin calcium and release of the compositions' components at various pH levels. “% w/w” indicates percentage per tablet. The composition of Formulation 1 is described in the “Formula 4” column of Table 23.

Enteric coated Atorvastatin tablets targeting release of the drug substance at a pH of 7.0 were prepared as batch 121-16005.

These tablets were designed such that they could be inserted into a standard size 0 capsule shell for combination administration with gemcabene and optionally additional components, in which gemcabene and optionally additional components are individually and subsequently filled in the capsule. The tablet cores were prepared as a high-shear wet granulation using a lab-scale high-shear granulator (Vector/Freund GMX-LabMini). The granulation was passed through a #10 mesh sieve and subsequently dried in a lab-scale fluid-bed dryer (Vector/Freund MFL-01) to a final loss on drying (LOD) value of <2% as determined by a moisture balance. Dried granules were sieved through a #16 mesh sieve and blended with croscarmellose sodium and magnesium stearate in a diffusional blender (PK V-Blender). The final blend was compressed into tablets using ¼″ (6.35 mm) tablet tooling on a lab-scale rotary tablet press (Dynamic Exim 10 station tablet press). Tablets were then charged into a lab-scale fully perforated coating pan (Vector/Freund LDCS pan coater) and a Hypromellose sub-coating (Opadry Clear, Colorcon) was applied at a 3% weight gain, relative to the tablet core weight, to provide a barrier between the slightly alkaline tablet core and the pH sensitive enteric coating. Table 8 below describes the batch composition.

TABLE 8
Formulation of Core Composition and Batch Size of the Atorvastatin
Tablet Cores

| | | | | |
|---|---|---|---|---|
| Item | | Concentration | Mg/ | Amount/ |
| No. | Ingredient | % w/w | Tablet | Batch (g) |

Intra-Granular Ingredients

| | | | | |
|---|---|---|---|---|
| 1 | Atorvastatin Calcium | 13.67 | 21.87 | 272.375 |
| | (Trihydrate) | | | |
| 2 | Calcium Carbonate | 22.5 | 36.0 | 450.0 |
| 3 | Lactose Monohydrate | 40.08 | 64.13 | 801.625 |
| | (Tabletose 70) | | | |
| 4 | Microcrystalline Cellulose | 18.75 | 30.0 | 375.0 |
| | (PH102) | | | |
| 5 | Polyvinylpyrrolidone | 1.88 | 3.0 | 37.5 |
| | (Plasdone K29/32) | | | |
| 6 | Polysorbate 80 | 0.25 | 0.4 | 5.0 |
| 7 | Water, Purified | — | — | 357.5 |

Extra-Granular Ingredients

| | | | | |
|---|---|---|---|---|
| 8 | Croscarmellose Sodium | 2.5 | 4.0 | 50.0 |
| 9 | Magnesium stearate | 0.38 | 0.6 | 7.5 |

| | | | |
|---|---|---|---|
| Total | 100.0 | 160.0 | 2000.0 |

% w/w is calculated as percentage per core

An enteric coating comprising a combination of (1) Methacrylic acid, Methyl Acrylate and Methacrylate polymer (EUDRAGIT® FS30D) and (2) Methacrylic Acid Copolymer Type C (EUDRAGIT® L30D55) was then applied to the sub-coated tablet cores at a target 10% weight gain, relative to the uncoated tablet core, using a lab-scale fully perforated coating pan (Vector/Freund LDCS pan coater). The enteric coating composition is described in Table 9 and in Tables 23 and 24.

TABLE 9
Enteric Coating of the Atorvastatin Minitablets Formulation 1

| Item | | | | Amount/ |
|---|---|---|---|---|
| No. | Ingredient | % (W/W)¹ | Mg/Tablet | Batch (g) |
| 1 | Atorvastatin Tablets, | 88.5 | 160.0 | 1000.0 |
| | 20 mg | | | |

Sub-Coat Formula

| | | | | |
|---|---|---|---|---|
| 2 | Purified Water, USP | n/a | n/a | 405.0 |
| 3 | Opadry (Clear) | 2.65 | 4.8 | 45.0 |

Enteric-Coat Formula

| | | | | |
|---|---|---|---|---|
| 4 | Eudragit ® FS30D | 3.77 | 6.8² | 333.3 |
| 5 | Eudragit ® L30D55 | 1.89 | 3.4² | 166.7 |
| 6 | Triethyl Citrate, NF | 0.39 | 0.7 | 10.0 |
| 7 | Talc USP/EP (Luzenac | 2.82 | 5.1 | 75.0 |
| | Pharma M) | | | |
| 8 | Purified Water, USP | n/a | n/a | 590.0 |
| | Total | 100.0 | 180.8 | |

Dissolution Profile:

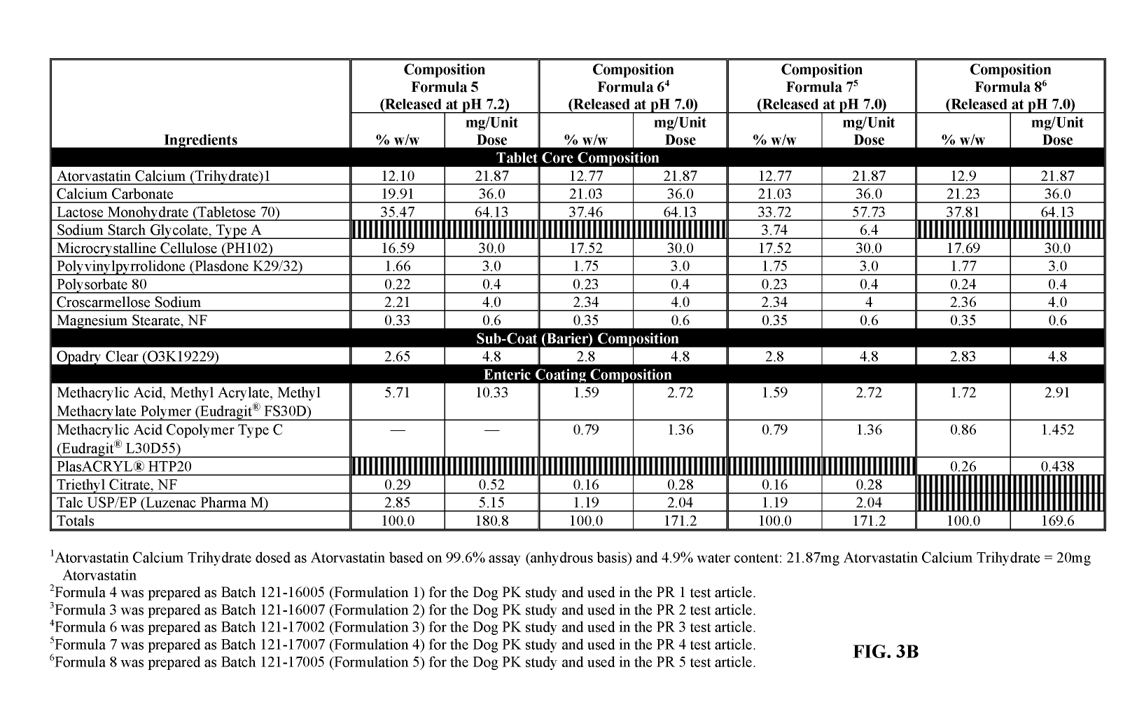

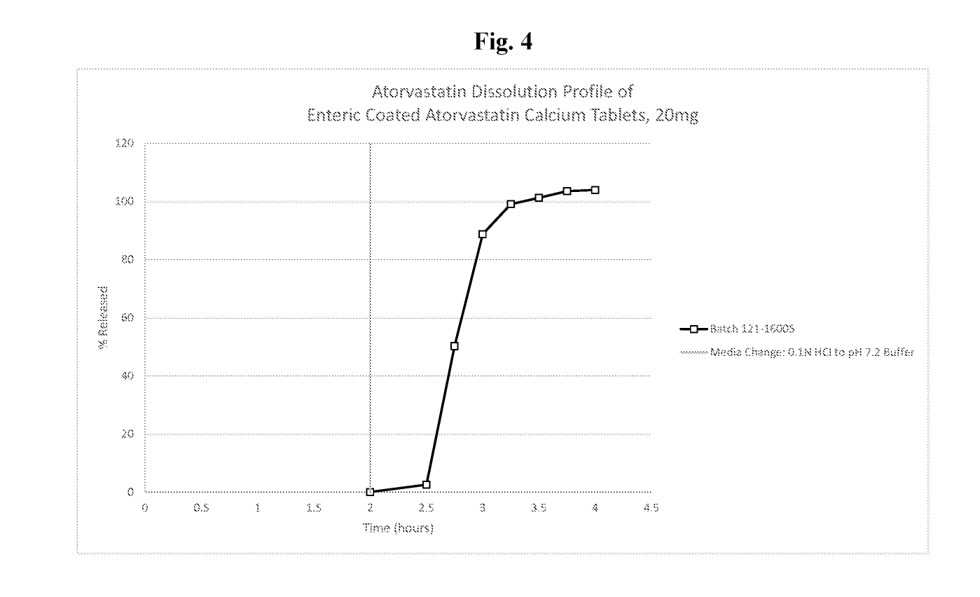

The dissolution testing was performed per USP <711> Delayed Release Dosage Forms Method A using Apparatus 2 (paddles) at 100 RPM (FIG. 4). Stage 1 dissolution medium was 0.1N HCl, and after 2 hours 250 mL of sodium phosphate buffer solution was added to adjust medium's pH to 7.2. Samples collected for analysis after 2 hours in the acid stage and then at 30, 45, 60, 75, 90, 105, and 120 minutes post media change.

### Example 6: Atorvastatin Calcium Formulation 2—Used in PR 2 Test Article in Dog Pharmacokinetic Study

The composition of Formulation 2 is described in the “Formula 3” column of Table 23.

Formulation 2 was prepared as described above for Formulation 1, using the same granulated blend as for Formulation 1. The same tableting parameters and Hypromellose sub-coating used for Formulation 1 were applied to these tablets.

An enteric coating comprising a combination of (1) Methacrylic acid, Methyl Acrylate and Methacrylate polymer (EUDRAGIT® FS30D) and (2) Methacrylic Acid Copolymer Type C (EUDRAGIT® L30D55) was then applied to the sub-coated tablet cores at a target 10% weight gain, relative to the uncoated tablet core, using a lab-scale fully perforated coating pan (Vector/Freund LDCS pan coater). The enteric coating composition is described in Table 10 and in Tables 23 and 24.

TABLE 10
Enteric Coating of the Atorvastatin Minitablets Formulation 2

| Item | | | | Amount/ |
|---|---|---|---|---|
| No. | Ingredient | % (W/W)¹ | Mg/Tablet | Batch (g) |
| 1 | Atorvastatin Tablets, | 88.5 | 160.0 | 1000.0 |
| | 20 mg | | | |

Sub-Coat Formula

| | | | | |
|---|---|---|---|---|
| 2 | Purified Water, USP | n/a | n/a | 405.0 |
| 3 | Opadry (Clear) | 2.65 | 4.8 | 45.0 |

Enteric-Coat Formula

| | | | | |
|---|---|---|---|---|
| 4 | Eudragit ® FS30D | 2.45 | 6.8² | 333.3 |
| 5 | Eudragit ® L30D55 | 3.20 | 3.4² | 166.7 |
| 6 | Triethyl Citrate, NF | 0.39 | 0.7 | 10.0 |
| 7 | Talc USP/EP (Luzenac | 2.82 | 5.1 | 75.0 |
| | Pharma M) | | | |
| 8 | Purified Water, USP | n/a | n/a | 590.0 |
| | Total | 100.0 | 180.8 | |

Dissolution Profile:

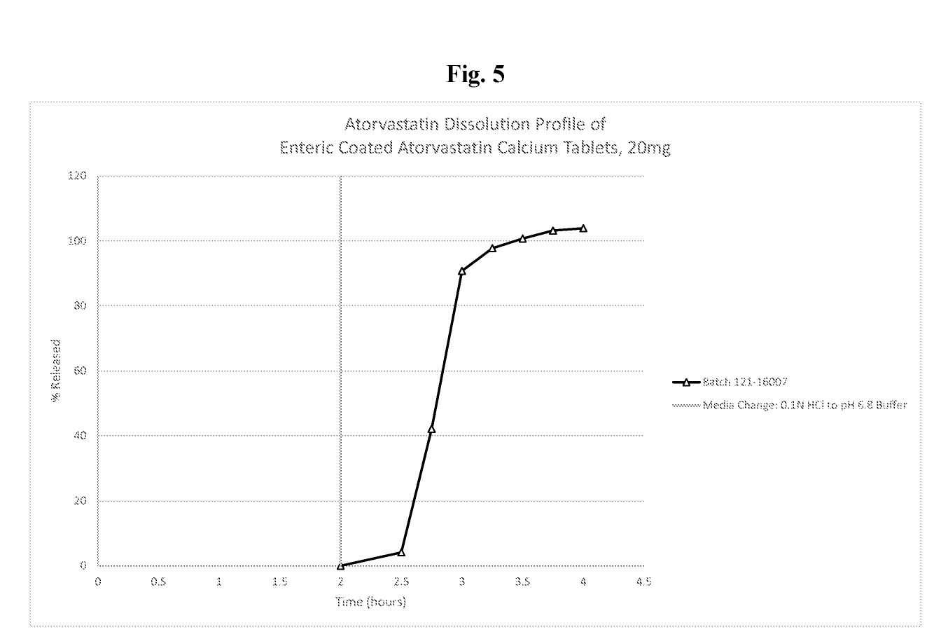

The dissolution testing was performed per USP <711> Delayed Release Dosage Forms Method A using Apparatus 2 (paddles) at 100 RPM. Stage 1 dissolution medium was 0.1N HCl, and after 2 hours 250 mL of sodium phosphate buffer solution was added to adjust medium's pH to 6.8. Samples collected for analysis after 2 hours in the acid stage and then at 30, 45, 60, 75, 90, 105, and 120 minutes post media change (FIG. 5).

### Example 7: Atorvastatin Calcium Formulation 3—Used in PR 3 Test Article in Dog Pharmacokinetic Study

The composition of Formulation 3 is described in the “Formula 6” column of Table 24.

Formulation 3 was prepared as described above for Formulation 1, using the same granulated blend as for Formulation 1. The same tableting parameters and Hypromellose sub-coating used for Formulation 1 were applied to these tablets.

An enteric coating comprising a combination of (1) Methacrylic acid, Methyl Acrylate and Methacrylate polymer (EUDRAGIT® FS30D) and (2) Methacrylic Acid Copolymer Type C (EUDRAGIT® L30D55) was then applied to the sub-coated tablet cores at a target 4% weight gain, relative to the uncoated tablet core, using a lab-scale fully perforated coating pan (Vector/Freund LDCS pan coater). The enteric coating composition is described in Table 11 and in Tables 23 and 24.

TABLE 11
Enteric Coating of the Atorvastatin Minitablets Formulation 3

| Item | | | | Amount/ |
|---|---|---|---|---|
| No. | Ingredient | % (W/W) | Mg/Tablet | Batch (g) |
| 1 | Atorvastatin Tablets, | 93.46 | 160.0 | 1000.0 |
| | 20 mg | | | |

Sub-Coat Formula

| | | | | |
|---|---|---|---|---|
| 2 | Purified Water, USP | n/a | n/a | 450.0 |
| 3 | Opadry (Clear) | 2.80 | 4.8 | 50.0 |

Enteric-Coat Formula

| | | | | |
|---|---|---|---|---|
| 4 | Eudragit ® FS30D | 1.59 | 2.72 | 333.3 |
| 5 | Eudragit ® L30D55 | 0.79 | 1.36 | 166.7 |
| 6 | Triethyl Citrate, NF | 0.16 | 0.28 | 10.0 |
| 7 | Talc USP/EP (Luzenac | 1.19 | 2.04 | 75.0 |
| | Pharma M) | | | |
| 8 | Purified Water, USP | n/a | n/a | 590.0 |
| | Total | 100.0 | 171.2 | |

Dissolution Profile:

The dissolution testing was performed per USP <711> Delayed Release Dosage Forms Method A using Apparatus 2 (paddles) at 100 RPM. Stage 1 dissolution medium was 0.1N HCl, and after 2 hours 250 mL of sodium phosphate buffer solution was added to adjust medium's pH to 7.2. Samples collected for analysis after 2 hours in the acid stage and then at 30, 45, 60, 75, 90, 105, and 120 minutes post media change (FIG. 6).

### Example 8: Atorvastatin Calcium Formulation 4—Used in PR 4 Test Article in Dog Pharmacokinetic Study

The composition of Formulation 4 is described in the “Formula 7” column of Table 24.

Formulation 4 was prepared as described above for Formulation 1, using the same granulated blend as for Formulation 1, with the exception of the inclusion of Sodium Starch Glycolate, Type A in the granulation formulation. The same tableting parameters and Hypromellose sub-coating used for Formulation 1 were applied to these tablets.

An enteric coating comprising a combination of (1) Methacrylic acid, Methyl Acrylate and Methacrylate polymer (EUDRAGIT® FS30D) and (2) Methacrylic Acid Copolymer Type C (EUDRAGIT® L30D55) was then applied to the sub-coated tablet cores at a target 10% weight gain, relative to the uncoated tablet core, using a lab-scale fully perforated coating pan (Vector/Freund LDCS pan coater). The enteric coating composition is described in Table 12 and in Tables 23 and 24.

TABLE 12
Enteric Coating of the Atorvastatin Minitablets Formulation 4

| Item | | | | Amount/ |
|---|---|---|---|---|
| No. | Ingredient | % (W/W) | Mg/Tablet | Batch (g) |
| 1 | Atorvastatin Tablets, | 93.46 | 160.0 | 1000.0 |
| | 20 mg | | | |

Sub-Coat Formula

| | | | | |
|---|---|---|---|---|
| 2 | Purified Water, USP | n/a | n/a | 405.0 |
| 3 | Opadry (Clear) | 2.80 | 4.8 | 45.0 |

Enteric-Coat Formula

| | | | | |
|---|---|---|---|---|
| 4 | Eudragit ® FS30D | 1.59 | 2.72 | 333.3 |
| 5 | Eudragit ® L30D55 | 0.79 | 1.36 | 166.7 |
| 6 | Triethyl Citrate, NF | 0.16 | 0.28 | 10.0 |
| 7 | Talc USP/EP (Luzenac | 1.19 | 2.04 | 75.0 |
| | Pharma M) | | | |
| 8 | Purified Water, USP | n/a | n/a | 590.0 |
| | Total | 100.0 | 171.2 | |

Dissolution Profile:

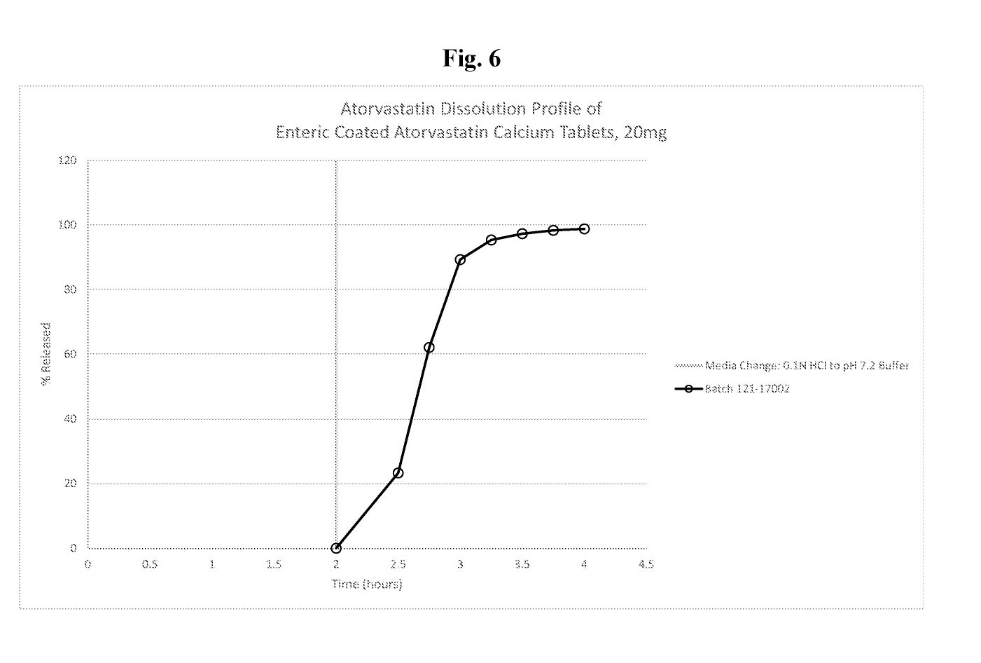

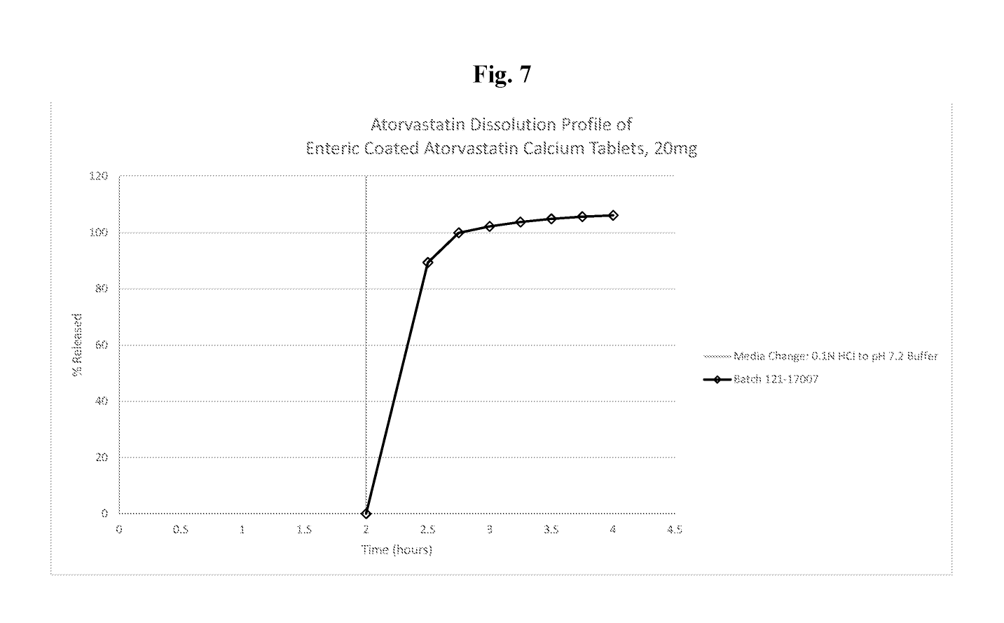

The dissolution testing was performed according to USP <711> Delayed Release Dosage Forms Method A using Apparatus 2 (paddles) at 100 RPM. Stage 1 dissolution medium was 0.1N HCl, and after 2 hours 250 mL of sodium phosphate buffer solution was added to adjust medium's pH to 7.2. Samples collected for analysis after 2 hours in the acid stage and then at 30, 45, 60, 75, 90, 105, and 120 minutes post media change (FIG. 7).

### Example 9: Atorvastatin Calcium Formulation 5—Used in PR 5 Test Article in Dog Pharmacokinetic Study

The composition of Formulation 5 is described in the “Formula 8” column of Table 24.

Formulation 5 was prepared as described above for Formulation 1, using the same granulated blend as for Formulation 1. The same tableting parameters and Hypromellose sub-coating used for Formulation 1 were applied to these tablets.

An enteric coating comprising a combination of (1) Methacrylic acid, Methyl Acrylate and Methacrylate polymer (EUDRAGIT® FS30D) and (2) Methacrylic Acid Copolymer Type C (EUDRAGIT® L30D55) was then applied to the sub-coated tablet cores at a target 3% weight gain, relative to the uncoated tablet core, using a lab-scale fully perforated coating pan (Vector/Freund LDCS pan coater). The enteric coating composition is described in Table 13 and in Tables 23 and 24.

TABLE 13
Enteric Coating of the Atorvastatin Minitablets Formulation 5

| Item | | | | Amount/ |
|---|---|---|---|---|
| No. | Ingredient | % (W/W) | Mg/Tablet | Batch (g) |
| 1 | Atorvastatin Tablets, | 94.34 | 160.0 | 1000.0 |
| | 20 mg | | | |

Sub-Coat Formula

| | | | | |
|---|---|---|---|---|
| 2 | Purified Water, USP | n/a | n/a | 405.0 |
| 3 | Opadry (Clear) | 2.83 | 4.8 | 45.0 |

Enteric-Coat Formula

| | | | | |
|---|---|---|---|---|
| 4 | Eudragit ® FS30D | 1.72 | 291 | 333.3 |
| 5 | Eudragit ® L30D55 | 0.86 | 1.452 | 166.7 |
| 6 | PlasAcryl HTP20 | 0.26 | 0.438 | 75.0 |
| 8 | Purified Water, USP | n/a | n/a | 250.0 |
| | Total | 100.0 | 169.6 | |

Dissolution Profile:

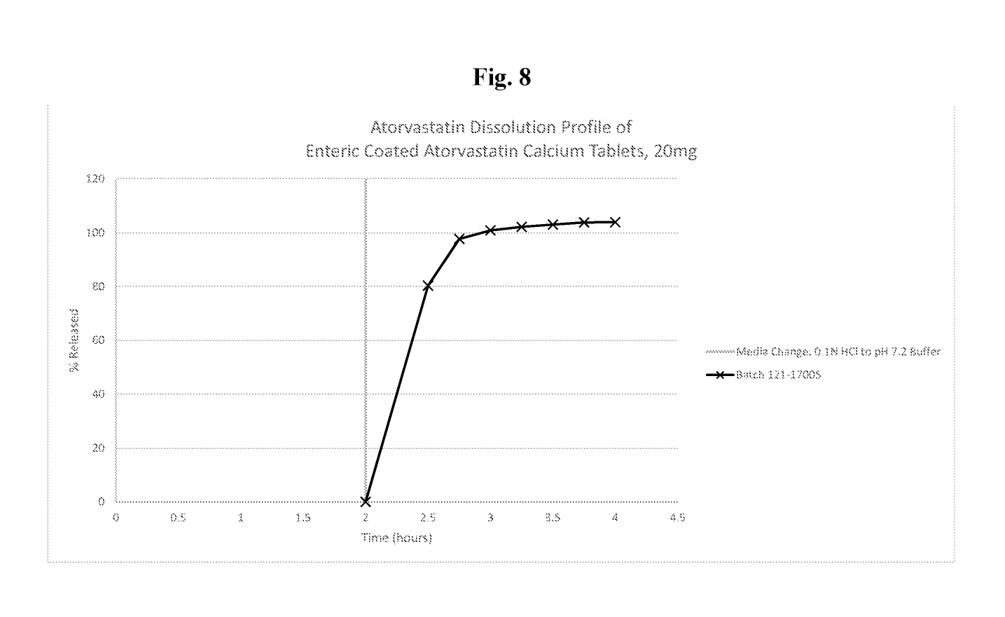

The dissolution testing was performed according to USP <711> Delayed Release Dosage Forms Method A using Apparatus 2 (paddles) at 100 RPM. Stage 1 dissolution medium was 0.1N HCl, and after 2 hours 250 mL of sodium phosphate buffer solution was added to adjust medium's pH to 7.2. Samples collected for analysis after 2 hours in the acid stage and then at 30, 45, 60, 75, 90, 105, and 120 minutes post media change (FIG. 8).

### Example 10: Pharmacokinetic Analysis after Administration to Male Dogs of a Single Oral Dose of (1) Atorvastatin Calcium, (2) Gemcabene Calcium or (3) Both Atorvastatin Calcium and Gemcabene Calcium

Plasma samples were collected in order to determine the pharmacokinetics (PK) of atorvastatin and gemcabene administered in experimental composition formulations in a single oral dose to male dogs. This study was conducted in accordance with the applicable Covance Laboratories Inc., Greenfield, Ind. (USA) standard operating procedures (SOPs), in a non-glp (Good Laboratory Practice Regulations) fashion. All procedures in the protocol were in compliance with the Animal Welfare Act Regulations (9 CFR 3).

The study was performed in 6 month-old to 3 year-old, male, drug naïve, purebred beagle dogs of 7 to 15 kg weight from the Covance stock colony. Animals were identified via individual cage cards, ear tag, tattoo, and implantable microchip identification devices (IMID), as applicable. Animals were housed in stainless steel cages, and were not commingled for at least 24 hours after experimental composition administration, to allow monitoring of any experimental composition-related effects. Also, animals were acclimated in the study room one day prior to treatment administration. Animals were not randomized, and were fed approximately 500 g per day of 2021, 21% Protein Dog Diet (Envigo RMS, Inc.) and/or Purina® Labdiet 5006, unless otherwise specified for dose administration. Greenfield city water was provided ad libitum. Animals were treated in accordance with the Animal Welfare Act, the Guide for the Care and Use of Laboratory Animals, and the Office of Laboratory Animal Welfare.

For Phases 1 through 4, four male purebred beagle dogs from the Covance stock colony were used. At the beginning of Phase 1, the animals were approximately 6 months of age.

For Phases 5 through 7, four male purebred beagle dogs from the Covance stock colony were used. At the beginning of Phase 5, the animals were approximately 13 months of age. In Phase 7, Dog C was not dosed.

For Phase 8, four male purebred beagle dogs from the Covance stock colony were used. At dosing, the animals were approximately 14 months of age.

The experimental compositions are presented in Table 14 below. All animals were fasted overnight through approximately 6 hours postdose. The capsule and tablet doses were administered orally by placing the capsule(s) or tablet(s) in the back of the throat and administering approximately 10 mL water to encourage the dog to swallow.

Table 14 describes the storage temperature and the composition of test articles administered to each dog during each test phase. The experimental design is provided in Table 15. Specifically, the doses were administered as follows:

(i) for Phase 1, individual doses were 20 mg atorvastatin calcium (Example 3)—1 capsule/animal;

(ii) for Phase 2, individual doses were 150 mg gemcabene calcium (Example 4)—1 capsule/animal;

(iii) for Phase 3, individual doses were a tablet-in-capsule formulation combination containing a 20 mg atorvastatin calcium tablet (Example 5) in 150 mg gemcabene calcium (Example 4)—1 tablet-in-capsule/animal;

(iv) for Phase 4, individual doses were a tablet-in-capsule formulation combination containing a 20 mg atorvastatin calcium tablet (Example 6) in 150 mg gemcabene calcium (Example 4)—1 tablet-in-capsule/animal;

(v) for Phase 5, individual doses were 40 mg LIPITOR® (atorvastatin calcium)—1 tablet/animal;

(vi) for Phase 6, individual doses were 20 mg atorvastatin calcium (Example 7)—2 tablets/animal;

(viii) for Phase 7, individual doses were 20 mg atorvastatin calcium (Example 8)—2 tablets/animal;

(viii) for Phase 8, individual doses were 20 mg atorvastatin calcium (Example 9)—2 tablets/animal.

TABLE 14
Test Articles

| Test Articles | Storage | Compositions |
|---|---|---|
| Atorvastatin | 15-30° C. | Atorvastatin Ca Capsule |
| Ca of Example 3 | | (Example 3), 20 mg active |
| Gemcabene | 15-30° C. | Gemcabene Ca Capsule |
| Ca of Example 4 | | (Example 4), 150 mg active |
| PR 1 | 15-30° C. | Atorvastatin Ca Tablet |
| | | (Formulation 1), 20 mg active in |
| | | Gemcabene Ca Capsule |
| | | (Example 4), 150 mg active |
| PR 2 | 15-30° C. | Atorvastatin Ca Tablet |
| | | (Formulation 2), 20 mg active in |
| | | Gemcabene Ca (Example 4) |
| | | Capsule, 150 mg active |
| Atorvastatin Ca | 15-30° C. | LIPITOR ® (atorvastatin |
| (commercial product) | | calcium) tablet 40 mg |
| PR 3 | 15-30° C. | Atorvastatin Ca (Formulation 3) |
| | | Tablet, 20 mg active × 2 (40 mg |
| | | total) |
| PR 4 | 15-30° C. | Atorvastatin Ca (Formulation 4) |
| | | Tablet, 20 mg active × 2 (40 mg |
| | | total) |
| PR 5 | 15-30° C. | Atorvastatin Ca (Formulation 5) |
| | | Tablet, 20 mg active × 2 (40 mg |
| | | total) |

TABLE 15
Experimental Design; Phase and Group Designations and Dose Levels

| | Number | | | Tarset Dose | Target Dose | Target Dose |
|---|---|---|---|---|---|---|
| Phase/ | of Male | | Dose | Level | Concentration | (capsule or |
| Group | Animals | Test Article | Route | (mg/animal) | (mg/formulation) | tablet/animal) |
| 1/1 | 4 | Atorvastatin Ca | PO | 20 | 20 | 1 |
| | | of Example 3 | | | | |
| 2/1 | 4 | Gemcabene Ca | PO | 150 | 150 | 1 |
| | | of Example 4 | | | | |
| 3/1 | 4 | PR 1 | PO | 20 + 150 | 20 + 150 | 1 + 1 |
| 4/1 | 4 | PR 2 | PO | 20 + 150 | 20 + 150 | 1 + 1 |
| 5/1 | 4 | LIPITOR ® | PO | 40 | 40 | 1 |
| | | (atorvastatin | | | | |
| | | calcium) | | | | |
| 6/1 | 4 | PR 3 | PO | 40 | 20 | 2 |
| 7/1 | 3 | PR 4 | PO | 40 | 20 | 2 |
| 8/1 | 4 | PR 5 | PO | 40 | 20 | 2 |

PO Oral.
Notes:
There was a 7-day washout period between Phases 1 and 2, a 12-day washout period between Phases 2 and 3, a 15-day washout period between Phases 3 and 4, an approximate 5.5-month washout period between Phases 4 and 5, a 7-day washout period between Phases 5 and 6, a 13-day washout period between Phases 6 and 7, and a 22-day washout period between Phases 7 and 8.

Animals were observed for mortality and signs of pain and distress at least once daily, and cage-side observations for general health and appearance were done once daily. Body Weights were recorded at the time of animal selection, on the day of dose administration. Detailed observations were performed on all available animals predose and 0.5, 2, 24 and 48 hours postdose for each phase. Upon completion of the in-life portion of the study, animals were returned to the Covance stock colony.

Sample collection.

For each phase, blood (approximately 3 mL) was collected via a jugular vein into tubes containing K2EDTA from each animal predose and at approximately 0.5, 1, 2, 4, 6, 12, 24, 36, 48, 60, 72, 96, 120, 144 and 168 hours postdose. Blood was maintained on wet ice or at approximately 5° C. prior to centrifugation to obtain plasma. Centrifugation began within 1 hour of collection. Resulting samples were harvested within 40 minutes of the start of centrifugation. Plasma samples were identified with the Covance study number, composition lot number, group, animal identification, phase, matrix, and collection time point or interval, and were placed into individually labeled 96-well tubes with barcodes, and maintained on dry ice prior to storage at ≤−60° C. Samples were analyzed for concentrations of Atorvastatin and Gemcabene at Medpace Bioanalytical Laboratories, Ohio (USA). Results were provided to Covance Laboratories Inc. for pharmacokinetic analysis.

### Pharmacokinetic Analysis.

Pharmacokinetic parameters were estimated using Phoenix® WinNonlin® version 6.4 or higher (Certara USA, Inc., Princeton, N.J.). A non-compartmental approach consistent with the oral route of administration will be used for parameter estimation. The individual plasma concentration-time data were used for pharmacokinetic calculations. In addition to parameter estimates for individual animals, descriptive statistics (e.g. mean, standard deviation, coefficient of variation) were reported, as appropriate. All parameters were generated from individual composition and metabolite concentrations in plasma. Samples that are below the lower limit of quantitation were treated as zero for determination of descriptive statistics and pharmacokinetic analysis. Embedded values below the lower limit of quantitation were excluded from pharmacokinetic analysis. Parameters were estimated using nominal dose levels. Parameters were estimated using nominal sampling times; if bioanalytical sample collection deviations are documented, actual sampling times will be used at the affected time points. Pharmacokinetic parameters were calculated and presented in the units provided by the analytical laboratory. Bioanalytical data were used as received from the pharmacokinetic analysis and were presented in tables and figures in the units provided. Descriptive statistics and pharmacokinetic parameters were reported to three significant figures. Pharmacokinetic parameters calculated during the study are presented in Table 16.

Table 16 shows pharmacokinetic parameters calculated from the time course measurements of atorvastatin and each atorvastatin metabolite (atorvastatin lactone, 2-hydroxy atorvastatin, 2-hydroxy atorvastatin lactone, 4-hydroxy atorvastatin, and 4-hydroxy atorvastatin lactone) in the plasma of each dog, following the administration of compositions containing atorvastatin during each test phase.

TABLE 16
Pharmacokinetic Parameters Measured

| Parameter | Description |
|---|---|
| Cₘₐₓ | Maximum observed concentration |
| Tₘₐₓ | Time of maximum observed concentration |
| AUC₀₋ₜ | Area under the curve from time 0 to the time of the last |
| | measurable concentration, calculated using the linear |
| | trapezoidal rule. |
| AUC_{0-inf} | Area under the curve from time 0 to infinity, calculated |
| | as AUC_{0-inf} = AUC₀₋ₜ + Cₜ/λ_{z}, where Cₜ is the |
| | last observed quantifiable concentration and λ_{z} is |
| | the elimination rate constant estimated using log-linear |
| | regression during the terminal elimination phase. The |
| | number of points used in λ_{z} calculation was determined |
| | by visual inspection of the data describing the terminal |
| | phase. At least the last three time points with measurable |
| | values were used in λ_{z} calculation. Cₘₐₓ values were not |
| | included in λ_{z} calculation. |
| AUC_{0-inf} | Percentage of the area under the concentration-time curve |
| % extrap | from hour 0 to infinity determined by extrapolation: |
| t_{1/2} | Elimination half-life, calculated as ln(2)/λ_{z}. |
| Fᵣₑₗ | Relative bioavailability, calculated for atorvastatin and |
| | gemcabene as: [AUC_{0-inf} or AUC₀₋ₜ PR 1 or PR 2]/ |
| | [AUC_{0-inf} or AUC₀₋ₜ Reference] · [Dose |
| | Reference]/[Dose PR 1 or PR 2] and calculated for |
| | atorvastatin as [AUC_{0-inf} or AUC₀₋ₜ PRs 3, 4, and 5]/ |
| | [AUC_{0-inf} or AUC₀₋ₜ Lipitor] [Dose Lipitor]/ |
| | [Dose PRs 3, 4, and 5] |
| AUC | AUC ratio, calculated for atorvastatin metabolites as: |
| Ratio | [AUC_{0-inf} or AUC₀₋ₜ PR 1 or PR 2]/[AUC_{0-inf} or |
| | AUC₀₋ₜ Reference] and calculated for atorvastatin as |
| | [AUC_{0-inf} or AUC₀₋ₜ PRs 3, 4, and 5]/[AUC_{0-inf} |
| | or AUC₀₋ₜ Lipitor] |
| M:P | AUC₀₋ₜ 2-hydroxy atorvastatin, 2-hydroxy atorvastatin |
| | lactone, 4 hydroxy atorvastatin, 4-hydroxy atorvastatin |
| | lactone, and atorvastatin lactone]/[AUC₀₋ₜ |
| | atorvastatin] This parameter was calculated separately |
| | for LIPITOR ® (atorvastatin calcium) and each PR. |

Based on these parameters, the mass weight of atorvastatin and the sum of the two hydroxyl-atorvastatin active metabolites was compared with the mass weight of the three lactone inactive metabolites. Tables 17 and 18 present the % mass (which is identical to % mol/mol, given that molecular weights are essentially similar) of atorvastatin lactones at 24 hrs.

Table 17 summarizes the percent distribution between the atorvastatin non-lactones (atorvastatin parent, 2-hydroxy atorvastatin, 4-hydroxy atorvastatin) and atorvastatin lactones (atorvastatin lactone, 2-hydroxy atorvastatin lactone, 4-hydroxy atorvastatin lactone) and the time of appearance in the blood of any atorvastatin analyte for each dog for each study phase dogs received a formulation containing atorvastatin.

TABLE 17
Percent Atorvastatin Lactone and Non-Lactone Formation
for Dosing Phases in Four Test Canines

| | | | | |
|---|---|---|---|---|
| | | | | Time of release into |
| Phase | Atorvastatin | Non-Lactones | Lactones | blood after dosing (Hr) |

Canine A

| | | | | |
|---|---|---|---|---|
| 1 | 100.0% | 80.3% | 19.7% | 0.5 |
| 3 | 100.0% | 100.0% | 0.0% | 6 |
| 4 | 100.0% | 100.0% | 0.0% | 4 |
| 5 | 100.0% | 88.7% | 11.3% | 0.5 |
| 6 | 100.0% | 82.5% | 17.5% | 2 |
| 7 | 100.0% | 77.3% | 22.7% | 1 |

Canine B

| | | | | |
|---|---|---|---|---|
| 1 | 100.0% | 75.3% | 24.7% | 1 |
| 3 | 100.0% | 76.0% | 24.0% | 4 |
| 4 | 100.0% | 72.1% | 27.9% | 4 |
| 5 | 100.0% | 80.6% | 19.4% | 0.5 |
| 6 | 100.0% | 85.0% | 15.0% | 2 |
| 7 | 100.0% | 77.0% | 23.0% | 1 |

Canine C

| | | | | |
|---|---|---|---|---|
| 1 | 100.0% | 85.2% | 23.0% | 0.5 |
| 3 | 100.0% | 84.8% | 15.2% | 2 |
| 4 | 100.0% | 100.0% | 0.0% | 4 |
| 5 | 100.0% | 82.8% | 17.2% | 0.5 |
| 6 | 100.0% | 78.4% | 21.6% | 1 |

Canine D

| | | | | |
|---|---|---|---|---|
| 1 | 100.0% | 86.8% | 13.2% | 0.5 |
| 3 | 100.0% | 91.4% | 8.6% | 2 |
| 4 | 100.0% | 78.1% | 21.9% | 4 |
| 5 | 100.0% | 88.0% | 12.0% | 0.5 |
| 6 | 100.0% | 87.5% | 12.5% | 2 |
| 7 | 100.0% | 86.5% | 13.5% | 1 |

Table 18 is the sum of all atorvastatin analytes (atorvastatin non-lactones plus atorvastatin lactones), atorvastatin non-lactones (atorvastatin parent, 2-hydroxy atorvastatin, 4-hydroxy atorvastatin) and atorvastatin lactones (atorvastatin lactone, 2-hydroxy atorvastatin lactone, 4-hydroxy atorvastatin lactone) for all time points collected for each phase of the study and for each dog. Each time point was reported as ng/mL and each value in the table represents the sum for all the time points collected for each phase of the study for each dog (data in table 17 are derived from the data in Table 18).

TABLE 18
AUC Readings for Atorvastatin Lactone and Atorvastatin Non-
Lactone Blood Levels in the Four Test Canines (sum of the
mass (ng) of atorvastatin all metabolites, atorvastatin non-
lactones, and atorvastatin lactones for all time points)

| | | | |
|---|---|---|---|
| Canine A | Canine B | Canine C | Canine D |

Phase 1

| | | | | |
|---|---|---|---|---|
| Atorvastatin | 40.158 | 20.844 | 22.566 | 22.531 |
| Non-Lactones | 32.239 | 15.694 | 17.763 | 19.547 |
| Lactones | 7.919 | 5.15 | 4.803 | 2.984 |

Phase 3

| | | | | |
|---|---|---|---|---|
| Atorvastatin | 0.91 | 4.31 | 3.242 | 2.874 |
| Non-Lactones | 0.91 | 3.274 | 2.749 | 2.627 |
| Lactones | 0 | 1.036 | 0.493 | 0.247 |

Phase 4

| | | | | |
|---|---|---|---|---|
| Atorvastatin | 0.503 | 2.713 | 0.183 | 1.268 |
| Non-Lactones | 0.503 | 1.957 | 0.183 | 0.99 |
| Lactones | 0 | 0.756 | 0 | 0.278 |

Phase 5

| | | | | |
|---|---|---|---|---|
| Atorvastatin | 42.557 | 41.798 | 70.132 | 32.239 |
| Non-Lactones | 37.74 | 33.686 | 58.054 | 28.36 |
| Lactones | 4.817 | 8.112 | 12.078 | 3.879 |

Phase 6

| | | | | |
|---|---|---|---|---|
| Atorvastatin | 8.539 | 6.572 | 23.901 | 7.366 |
| Non-Lactones | 7.046 | 5.586 | 18.749 | 6.444 |
| Lactones | 1.493 | 0.986 | 5.152 | 0.922 |

Phase 7

| | | | | |
|---|---|---|---|---|
| Atorvastatin | 65.432 | 19.508 | Not Dosed | 14.299 |
| Non-Lactones | 50.575 | 15.017 | Not Dosed | 12.373 |
| Lactones | 14.857 | 4.491 | Not Dosed | 1.926 |

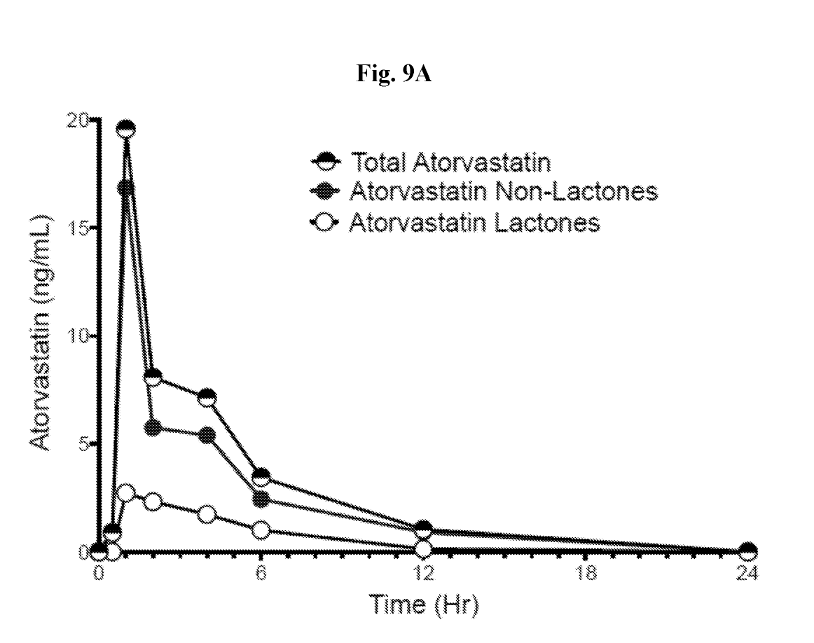

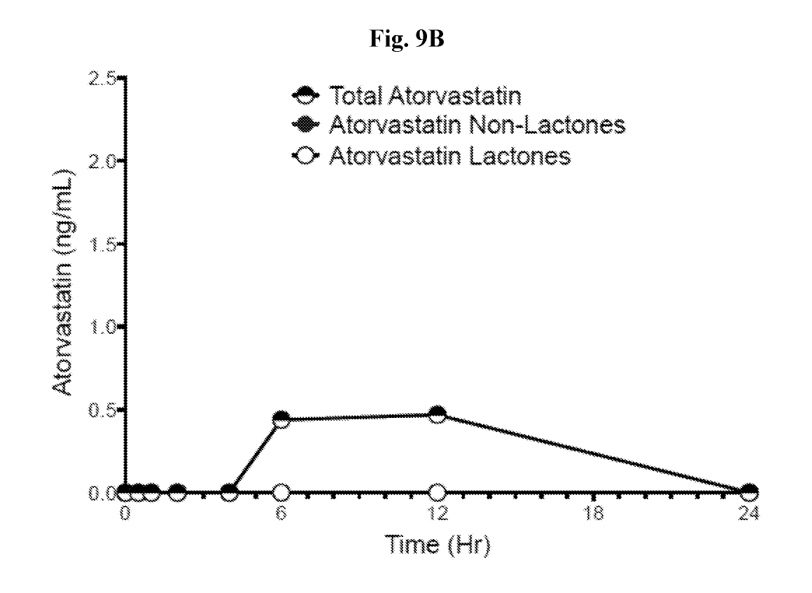

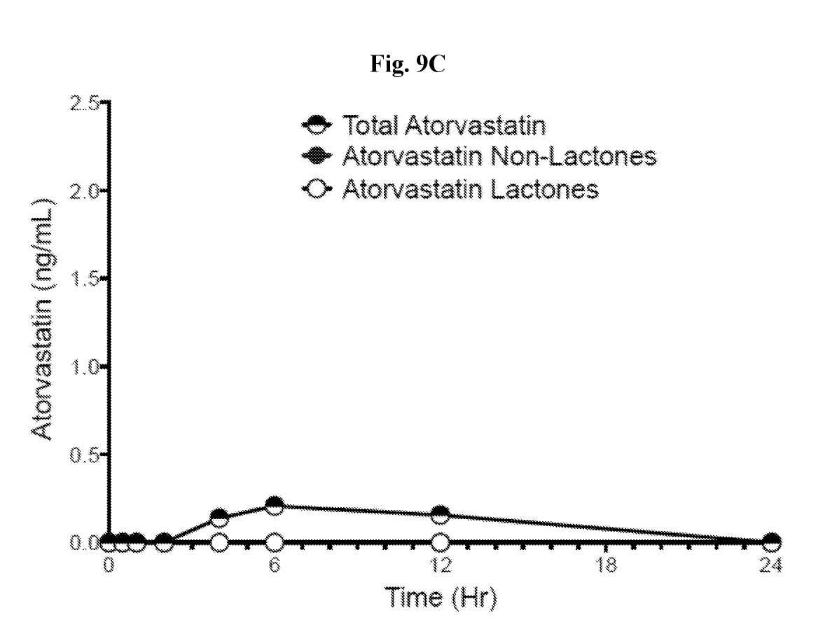

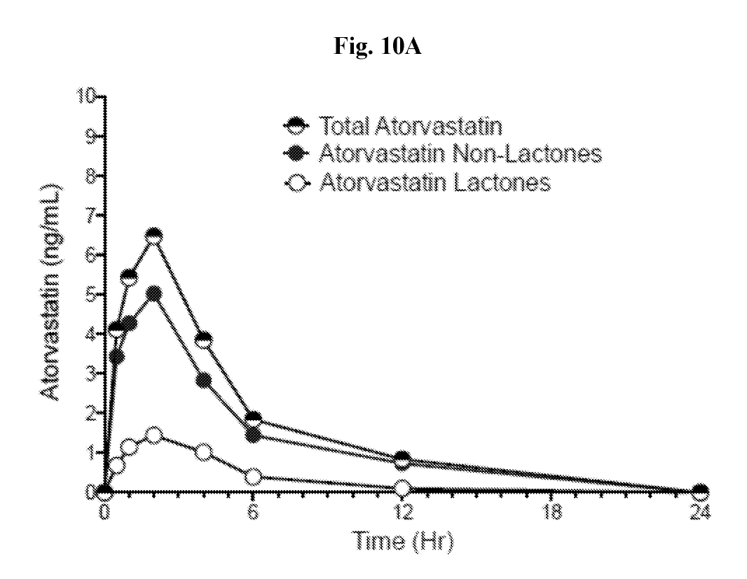

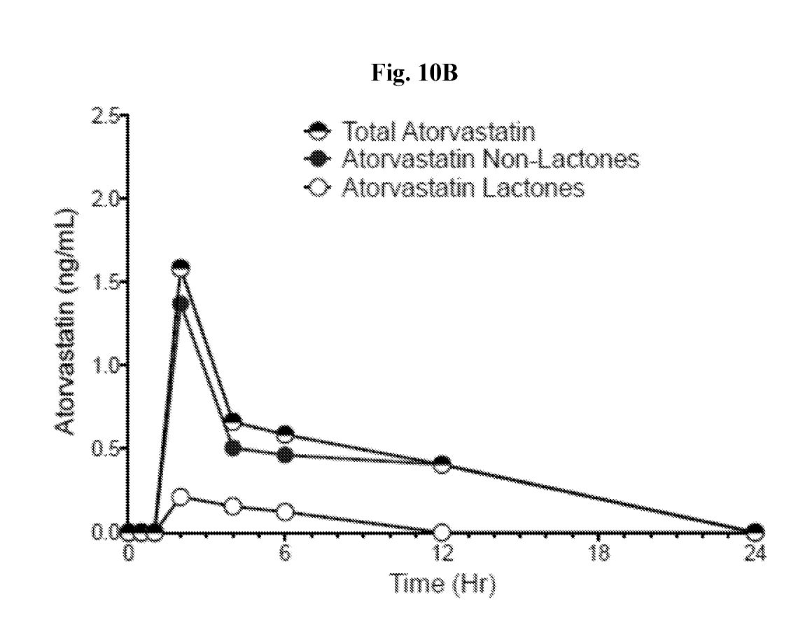

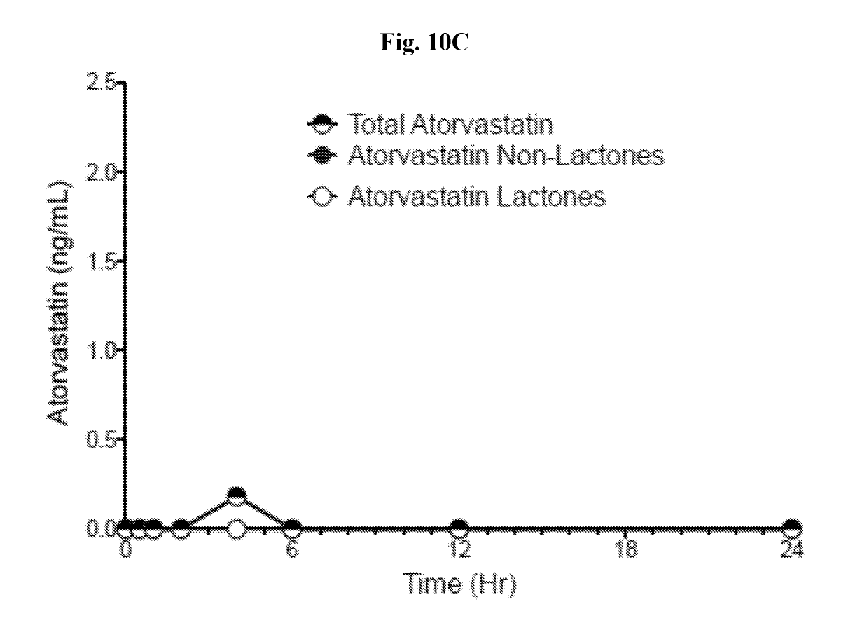

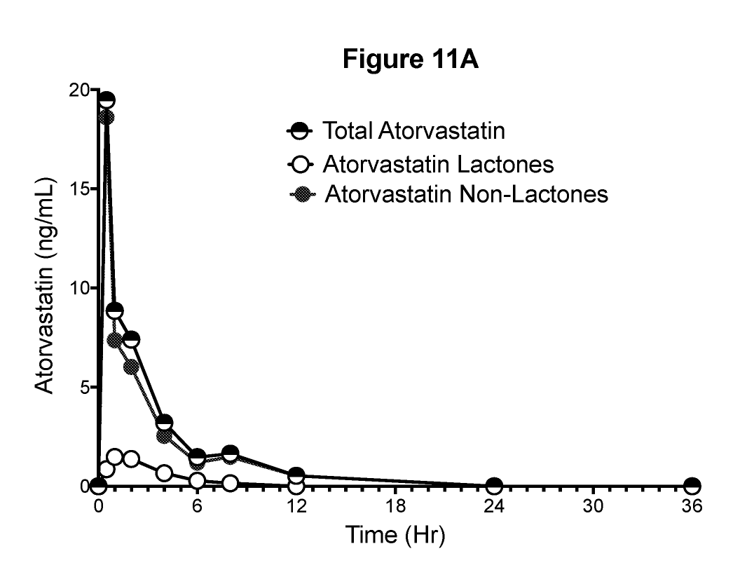

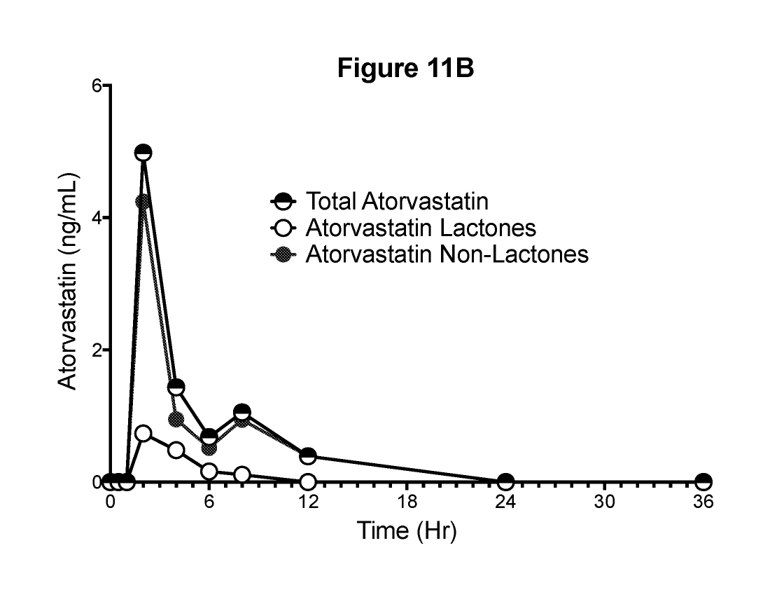

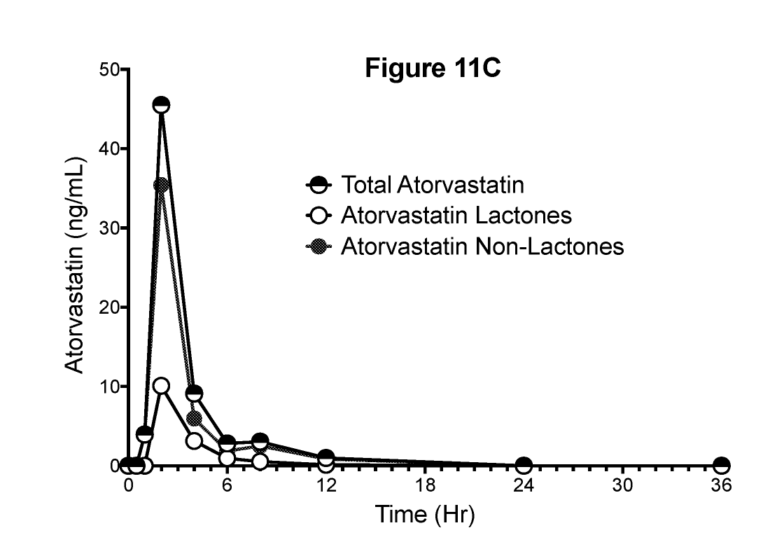

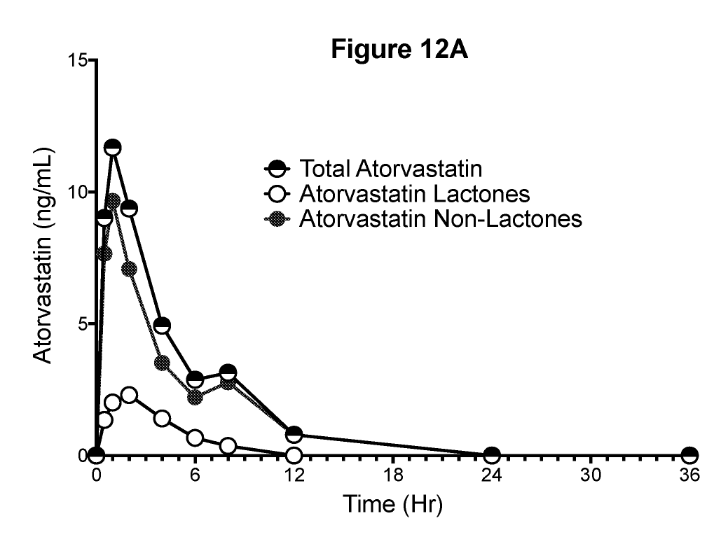

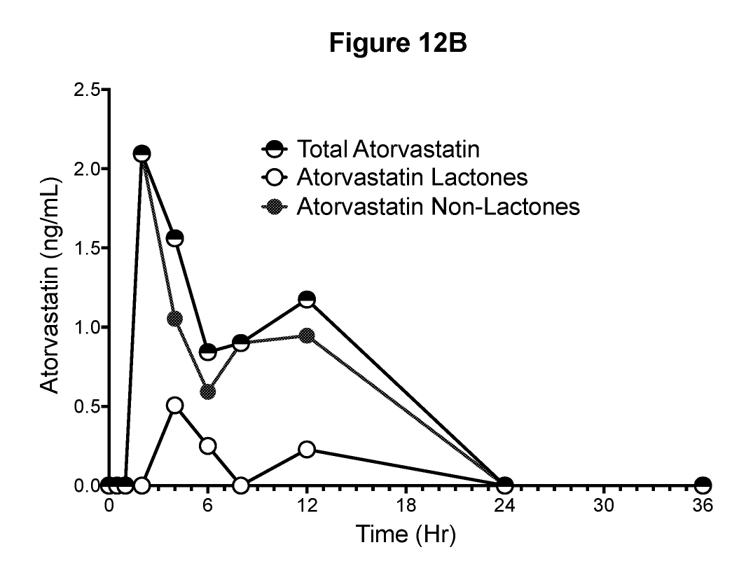

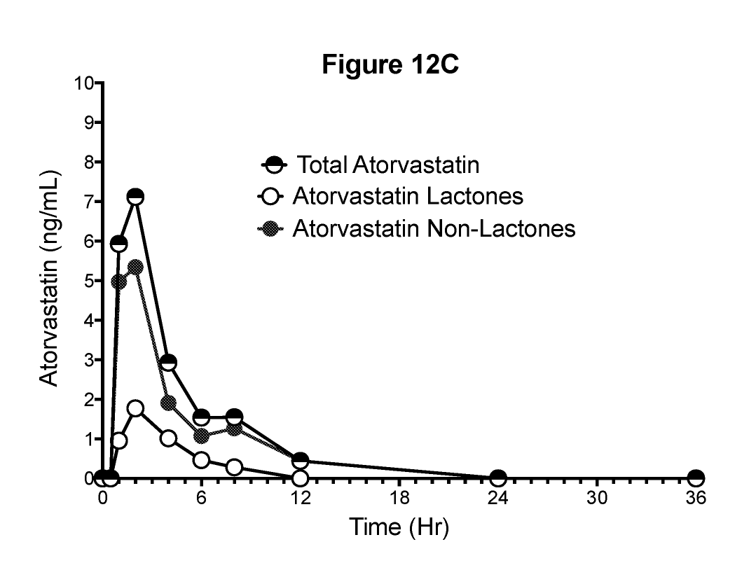

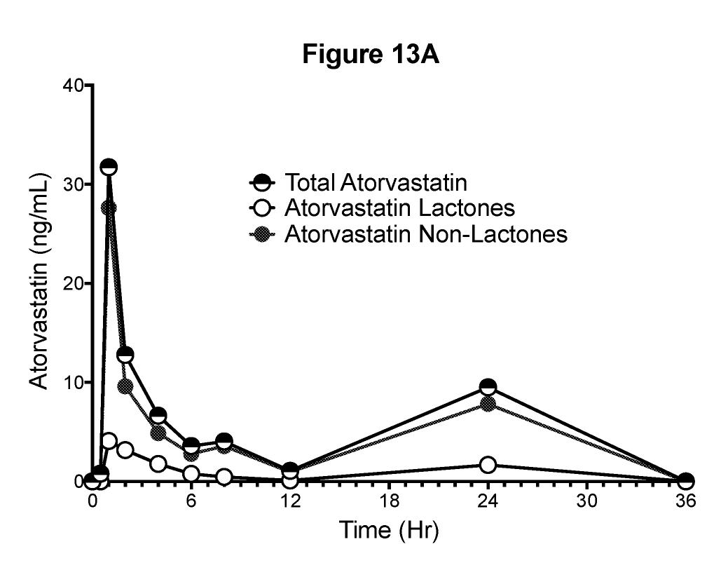

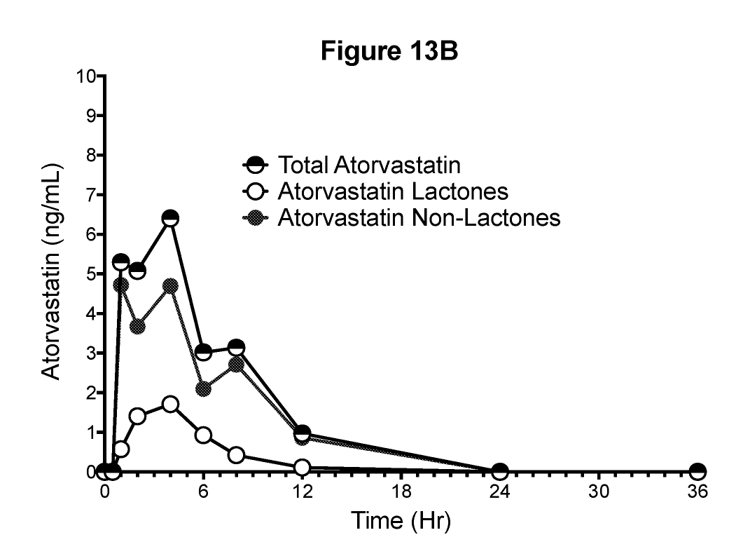

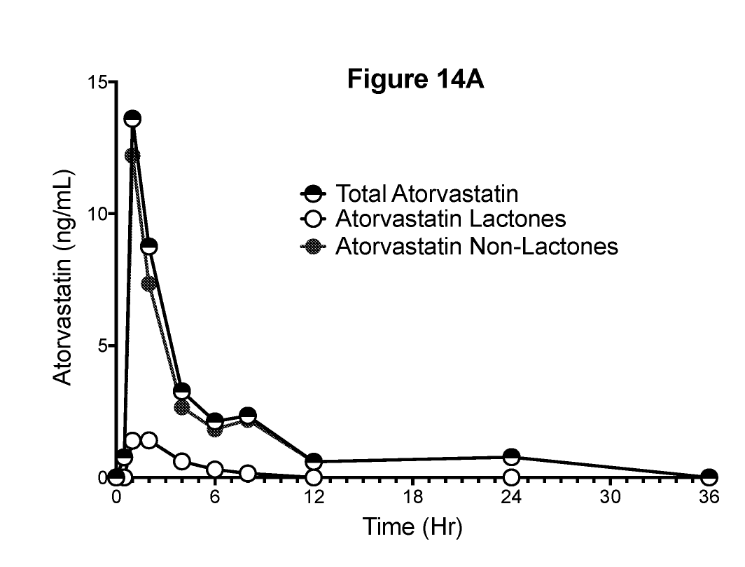

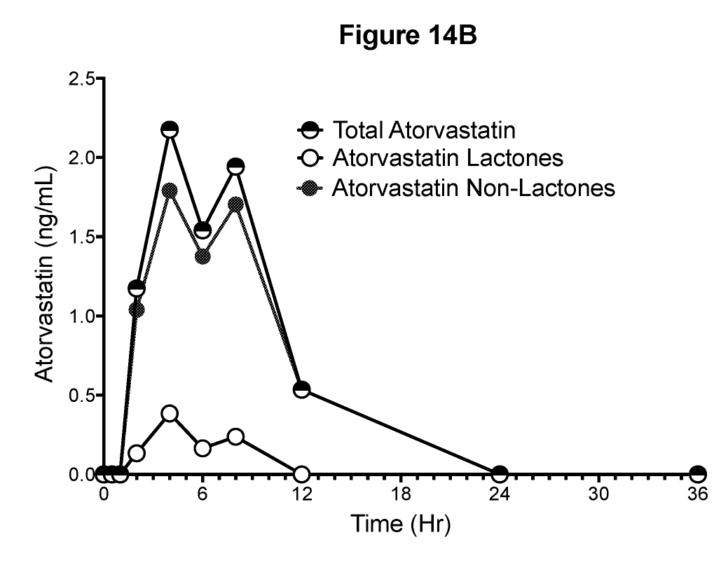

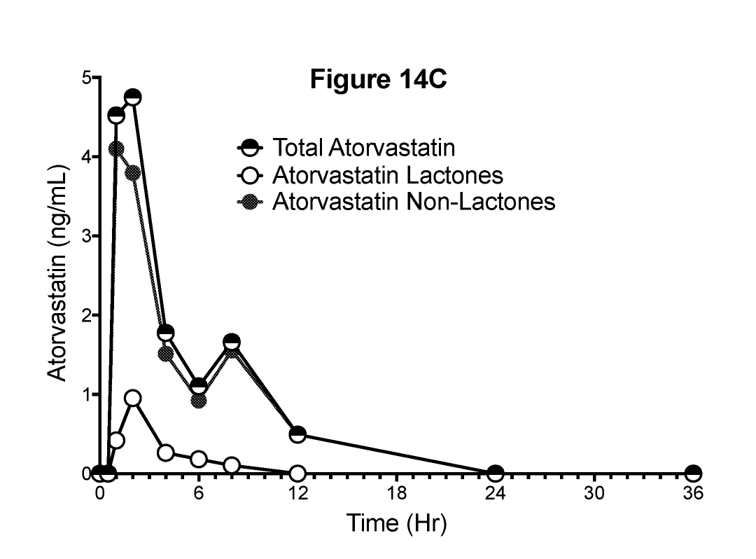

Pharmacokinetic profiles for canine A are provided in FIGS. 9A-9C and 11A-11C. Pharmacokinetic profiles for canine B are provided in FIGS. 12A-12C. Pharmacokinetic profiles for canine C are provided in FIGS. 10A-10C and 13A-13B. Pharmacokinetic profiles for canine D are provided in FIGS. 14A-14C.

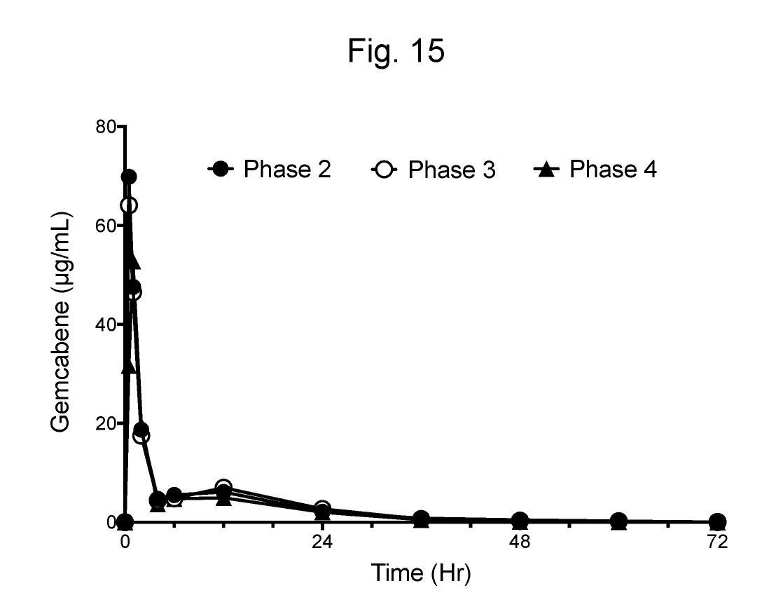

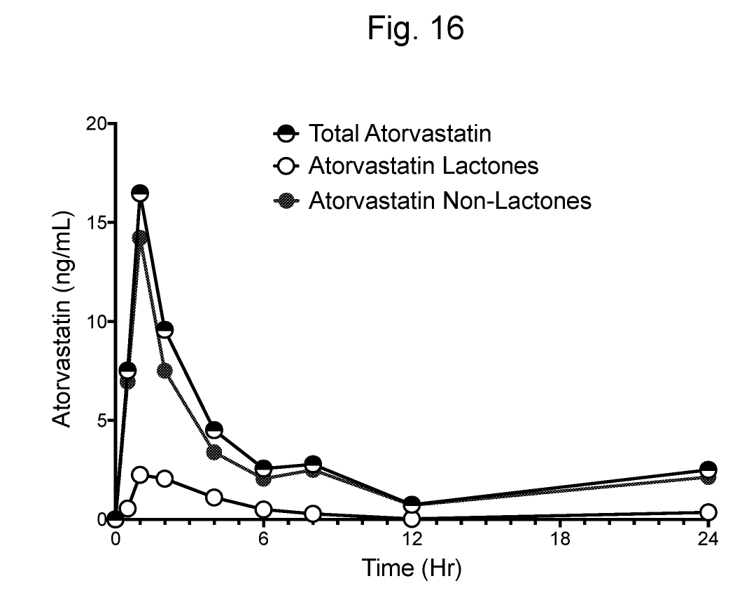

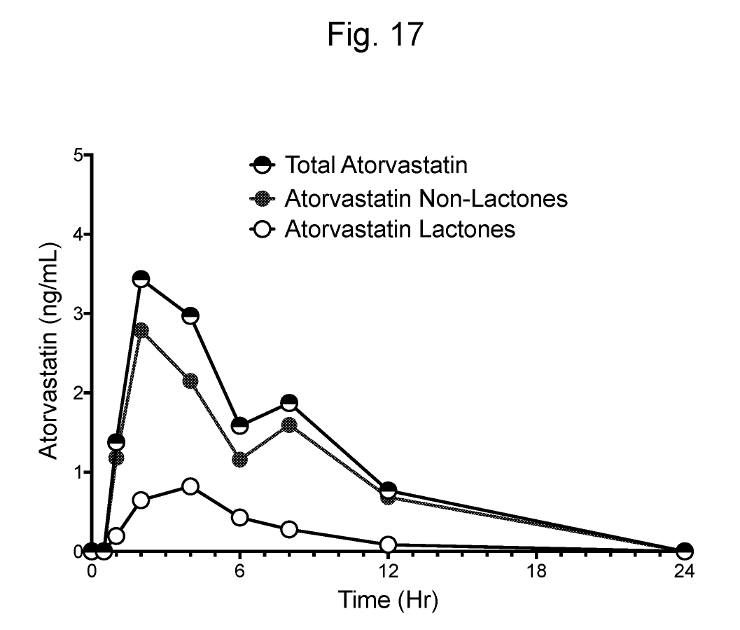

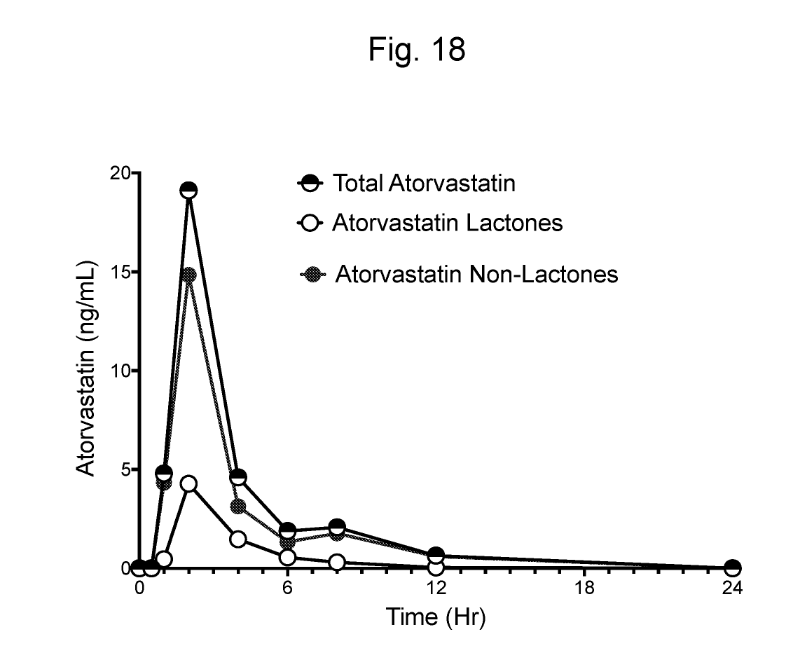

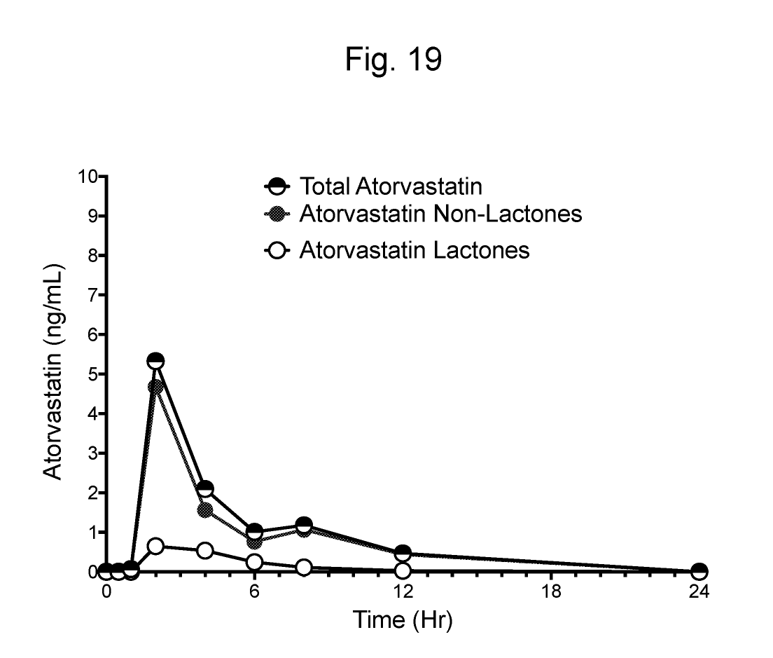

The pharmacokinetic profile of gemcabene when gemcabene calcium is administered in the absence of atorvastatin calcium and administered with atorvastatin calcium is essentially the same (FIG. 15), indicating a lack of drug-drug interaction during absorption.

A summary of the pharmacokinetic parameters calculated from Phases 1-8 is provided in Table 22. All atorvastatin PR test articles showed different metabolic profiles compared with generic atorvastatin calcium immediate release (Phase 1). The brand name Lipitor® showed a different metabolic profile than atorvastatin immediate release, with an increased percentage of lactone metabolites. All PR test articles of the invention showed a higher plasma concentration of total non-lactone metabolites and a lower plasma concentration of total lactone metabolites than Lipitor®. PR 2 (administered in Phase 4) and PR 5 (administered in Phase 8) shows a higher percentage of total non-lactone species (and correspondingly a lower percentage of total lactone species) than that produced by administration of other test articles. PR 5 shows an amount of total non-lactone species significantly lower than atorvastatin immediate release and Lipitor®. Hence, the modulation of the ratio of the pH-specific enteric coating polymer components of the atorvastatin PR test articles has impacted on the metabolic profile, with favorable impact on the total lactone metabolite ratio. Meanwhile, the talc component used in the composition of the enteric coating of PR 2 was replaced in PR 5 by the anti-adherent system PlasACRYL® HTP20, which may be connected to a more desirable decrease in the total non-lactone species in PR 5 compared to PR 2.

### Example 11: Microbeads of Atorvastatin Calcium and Gemcabene Calcium in Encapsulation Gemcabene Calcium Microbeads

Gemcabene calcium particles are coated using a spray-coating technique in a bottom-spray fluidized bed equipment. The coating suspension is prepared by mixing the coating excipients in an acetone/isopropyl alcohol mixture in a stainless steel vessel equipped with a stirring device. The suspension is sprayed at room temperature onto the gemcabene calcium particles in a fluidized bed apparatus working under nitrogen. During the process, the solvents are evaporated by the fluidization stream, allowing the composition to deposit around the particles as a continuous coating membrane, thus forming the gemcabene calcium microbeads.

The gemcabene calcium microbeads are mixed with the capsule filling excipients in order to obtain a free flowing blend. Optionally, a second pharmaceutically active ingredient formulated similarly to gemcabene calcium as microbeads is admixed with the capsule filling excipients into the blend. This blend is achieved in a drum-type blender of appropriate capacity. The resulting blend is used as a component in the fixed dose combination.

Examples of compositions of gemcabene calcium microbeads are indicated in Tables 19a-19c.

TABLE 19a
Illustrative Compositions of Gemcabene Calcium Dose

Composition A1

| | Composition | |
|---|---|---|
| Ingredient | (mg/capsule) | Composition (%) |
| Gemcabene | 151.00 | 94.70 |
| calcium | (gemcabene | |
| | diacid | |
| | equivalent) | |
| Ethylcellulose | 4.36 | 2.73 |
| Castor oil | 0.36 | 0.23 |
| Polyvinyl- | 0.24 | 0.15 |
| pyrolidone | | |
| Tartaric acid | 0.60 | 0.38 |
| Magnesium | 0.32 | 0.20 |
| stearate* | | |
| Anhydrous | 0.86 | 0.53 |
| colloidal | | |
| silica | | |
| Talc | 1.70 | 1.08 |
| Total | 159.44 | 100.00 |

TABLE 19b
Illustrative Compositions of Gemcabene Calcium Dose

Composition A2

| | Composition | |
|---|---|---|
| Ingredient | (mg/capsule) | Composition (%) |
| Gemcabene | 151.00 | 93.70 |
| calcium | (gemcabene | |
| | diacid | |
| | equivalent) | |
| Ethylcellulose | 5.78 | 3.60 |
| Castor oil | 0.24 | 0.15 |
| Polyvinyl- | 0.32 | 0.20 |
| pyrolidone | | |
| Tartaric acid | 0.80 | 0.50 |
| Magnesium | 0.42 | 0.25 |
| stearate* | | |
| Anhydrous | 0.86 | 0.53 |
| colloidal | | |
| silica | | |
| Talc | 1.72 | 1.07 |
| Total | 161.14 | 100.00 |

TABLE 19c
Illustrative Compositions of Gemcabene Calcium Dose

Composition A3

| | Composition | |
|---|---|---|
| Ingredient | (mg/capsule) | Composition (%) |
| Gemcabene | 151.00 | 92.20 |
| calcium | (gemcabene | |
| | diacid | |
| | equivalent) | |
| Ethylcellulose | 7.52 | 4.59 |
| Castor oil | 0.64 | 0.39 |
| Polyvinyl- | 0.42 | 0.26 |
| pyrolidone | | |
| Tartaric acid | 1.04 | 0.63 |
| Magnesium | 0.54 | 0.33 |
| stearate* | | |
| Anhydrous | 0.88 | 0.54 |
| colloidal | | |
| silica | | |
| Talc | 1.74 | 1.06 |
| Total | 163.78 | 100.00 |

### Atorvastatin Calcium Microbeads

Modified-release atorvastatin calcium batches with different lag times between swallowing and release starting point allowed selection of the targeted release sites. The products are obtained by coating atorvastatin calcium microbeads with a composition suitable for safe passage through the stomach after swallowing, then allowing release in different gastrointestinal tract segments. The product behavior (resistance in the stomach combined with release in a further specific location of GIT) is based on an association of three components in the coating composition: two hydrophilic methacrylic polymers, with different pH-dependent solubilities, and one hydrophobic material. It is inferred that the difference in lag times in vivo between the three formulae is determined by the different polymer ratios in the coating composition. Examples of compositions for atorvastatin microbead PRs are indicated in Tables 20a-20c. Different ratios of combinations of film coating agents insure the pH modulation from 6.5 to 7.5. Examples of w/w ratios between the two coating agents are described in Table 21.

The atorvastatin calcium microbeads are prepared as follows: atorvastatin calcium particles are coated using a spray-coating technique in a bottom-spray fluidized bed equipment. The coating solution is prepared by dissolving the coating excipients in hot isopropyl alcohol using a appropriate jacketed vessel equipped with a stirring device. The solution is sprayed at about 75° C. onto the atorvastatin granules, in the fluidized bed apparatus. During the process, the solvent is evaporated by the fluidization air stream, allowing the composition to deposit around the granules as a continuous coating membrane, thus forming microparticles. The atorvastatin calcium microbeads are mixed with the capsule filling excipients in a drum-type blender of appropriate capacity.

TABLE 20a
Examples of Compositions of Atorvastatin Calcium Microbeads

| | Composition B1 | | Composition B2 | |
|---|---|---|---|---|
| | (released in vitro at | | (released in vitro at | |
| | about pH 6) | | about pH 6.5) | |
| | mg/ | | mg/ | |
| Ingredient | capsule | % | capsule | % |
| Atorvastatin | 20.50 | 82.07 | 20.50 | 82.80 |
| Methacrylic Acid | 2.12 | 8.49 | 2.00 | 8.08 |
| Copolymer type C | | | | |
| (Eudragit ® L100- | | | | |
| 55) | | | | |
| Methacrylic Acid | 0.42 | 1.68 | 1.00 | 4.04 |
| Copolymer type B | | | | |
| (Eudragit ® S100) | | | | |
| Hydrogenated | 1.70 | 6.80 | 1.00 | 4.04 |
| Cottonseed Oil | | | | |
| (Lubritab ®) | | | | |
| Magnesium | 0.12 | 0.48 | 0.13 | 0.52 |
| stearate* | | | | |
| Colloidal Silicon | 0.12 | 0.48 | 0.13 | 0.52 |
| Dioxide | | | | |
| Total | 24.98 | 100.00 | 24.76 | 100.00 |

TABLE 20b
Examples of Compositions of Atorvastatin Calcium Microbeads

| | |
|---|---|
| Composition B3 | Composition B4 |
| (released in vitro | (released in vitro |
| at about pH 7) | at about pH 7) |

| | mg/ | | mg/ | |
|---|---|---|---|---|
| Ingredient | capsule | % | capsule | % |
| Atorvastatin | 20.50 | 79.58 | 20.50 | 78.97 |
| Methacrylic Acid | 1.00 | 3.88 | 0.80 | 3.08 |
| Copolymer type C | | | | |
| (Eudragit ® L100- | | | | |
| 55) | | | | |
| Methacrylic Acid | 2.00 | 7.77 | 2.40 | 9.24 |
| Copolymer type B | | | | |
| (Eudragit ® S100) | | | | |
| Hydrogenated | 2.00 | 7.77 | 2.00 | 7.71 |
| Cottonseed Oil | | | | |
| (Lubritab ®) | | | | |
| Magnesium | 0.13 | 0.50 | 0.13 | 0.50 |
| stearate* | | | | |
| Colloidal Silicon | 0.13 | 0.50 | 0.13 | 0.50 |
| Dioxide | | | | |
| Total | 25.76 | 100.00 | 25.96 | 100.00 |

TABLE 20c
Examples of Compositions of Atorvastatin Calcium Microbeads

Composition B5
(released in vitro at pH 7)

| | mg/ | |
|---|---|---|
| Ingredient | capsule | % |
| Atorvastatin | 20.50 | 79.58 |
| Methacrylic Acid | 0.25 | 0.97 |
| Copolymer type C | | |
| (Eudragit ® L100- | | |
| 55) | | |
| Methacrylic Acid | 2.75 | 10.68 |
| Copolymer type B | | |
| (Eudragit ® S100) | | |
| Hydrogenated | 2.00 | 7.77 |
| Cottonseed Oil | | |
| (Lubritab ®) | | |
| Magnesium | 0.13 | 0.50 |
| stearate* | | |
| Colloidal Silicon | 0.13 | 0.50 |
| Dioxide | | |
| Total | 25.76 | 100.00 |

TABLE 21
Examples of Mass Ratios of Enteric Coating Components
for Atorvastatin Calcium Microbeads

| | Dissolution | Atorvastatin Compositions | | | | |
|---|---|---|---|---|---|---|
| Enteric coating | pH | B1 | B2 | B3 | B4 | B5 |
| Methacrylic Acid | 6 | 5 | 2 | 1 | 1 | 1 |
| Copolymer type | | | | | | |
| C (Eudragit ® L100-55) | | | | | | |
| Methacrylic Acid | 7 | 1 | 1 | 2 | 4 | 10 |
| Copolymer type | | | | | | |
| B (Eudragit ® S100) | | | | | | |

### Encapsulation:

Admixtures of gemcabene calcium microbeads and filling excipients and atorvastatin calcium microbeads and filling excipients prepared as described above are admixed, and the admixture is placed into gelatin capsules.

### Examples 12A-12E: Tablet-in-Capsule Pharmaceutical Compositions

Example 12A—using PR 1. An atorvastatin tablet having a core as set forth in Table 8 of Example 5 and a coating as set forth in Table 9 of Example 5 is prepared. The gemcabene composition as set forth in Table 7 of Example 4 is prepared. A tablet-in-capsule oral dosage form containing the tablet and the composition is prepared using an automated encapsulator outfitted with a tablet inserter. Briefly, an empty capsule shell is loaded into a ring. The cap of the capsule shell is separated from the body of the capsule shell. The tablet inserter then inserts the tablet into the capsule shell. A dosator then back fills the capsule shell with a blend containing the gemcabene composition. The capsule cap is then locked into place and the finished capsule is ejected.

Example 12B—using PR 2. An atorvastatin tablet having a core as set forth in Table 8 of Example 5 and a coating as set forth in Table 10 of Example 6 is prepared. The gemcabene composition as set forth in Table 7 of Example 4 is prepared. A tablet-in-capsule oral dosage form containing the tablet and the composition is prepared using an automated encapsulator outfitted with a tablet inserter. Briefly, an empty capsule shell is loaded into a ring. The cap of the capsule shell is separated from the body of the capsule shell. The tablet inserter then inserts the tablet into the capsule shell. A dosator then back fills the capsule shell with a blend containing the gemcabene composition. The capsule cap is then locked into place and the finished capsule is ejected.

Example 12C—using PR 3. An atorvastatin tablet having a core as set forth in Table 8 of Example 5 and a coating as set forth in Table 11 of Example 7 is prepared. The gemcabene composition as set forth in Table 7 of Example 4 is prepared. A tablet-in-capsule oral dosage form containing the tablet and the composition is prepared using an automated encapsulator outfitted with a tablet inserter. Briefly, an empty capsule shell is loaded into a ring. The cap of the capsule shell is separated from the body of the capsule shell. The tablet inserter then inserts the tablet into the capsule shell. A dosator then back fills the capsule shell with a blend containing the gemcabene composition. The capsule cap is then locked into place and the finished capsule is ejected.

Example 12D—using PR 4. An atorvastatin tablet having a core as set forth in Table 8 of Example 5 and a coating as set forth in Table 12 of Example 8 is prepared. The gemcabene composition as set forth in Table 7 of Example 4 is prepared. A tablet-in-capsule oral dosage form containing the tablet and the composition is prepared using an automated encapsulator outfitted with a tablet inserter. Briefly, an empty capsule shell is loaded into a ring. The cap of the capsule shell is separated from the body of the capsule shell. The tablet inserter then inserts the tablet into the capsule shell. A dosator then back fills the capsule shell with a blend containing the gemcabene composition. The capsule cap is then locked into place and the finished capsule is ejected.

Example 12E—using PR 5. An atorvastatin tablet having a core as set forth in Table 8 of Example 5 and a coating as set forth in Table 13 of Example 9 is prepared. The gemcabene composition as set forth in Table 7 of Example 4 is prepared. A tablet-in-capsule oral dosage form containing the tablet and the composition is prepared using an automated encapsulator outfitted with a tablet inserter. Briefly, an empty capsule shell is loaded into a ring. The cap of the capsule shell is separated from the body of the capsule shell. The tablet inserter then inserts the tablet into the capsule shell. A dosator then back fills the capsule shell with a blend containing the gemcabene composition. The capsule cap is then locked into place and the finished capsule is ejected.

All publications and patents referred to in this disclosure are incorporated herein by reference to the same extent as if each individual publication or patent application were specifically and individually indicated to be incorporated by reference. Should the meaning of the terms in any of the patents or publications incorporated by reference conflict with the meaning of the terms bused in this disclosure, the meaning of the terms in this disclosure are intended to be controlling. Furthermore, the foregoing discussion discloses and describes merely exemplary embodiments of the present invention. One skilled in the art will readily recognize from such discussion and from the accompanying drawings and claims, that various changes, modifications and variations can be made therein without departing from the spirit and scope of the invention as defined in the following claims.

TABLE 22
Summary of pharmacokinetic parameters

| | | | | | Time for | AUC₀₋ₜ (ng · hr/mL) | | | |
|---|---|---|---|---|---|---|---|---|---|
| | | | | Atorva- | Atorvastatin to | | | | 2-Hydroxy |
| | | | Gemcabene | statin | First Appear | | Atorva- | 2-Hydroxy | Atorva- |
| | | Dogs | Dose | Dose | in Plasma | Atorva- | statin | Atorva- | statin |
| Phase | Test Article | (n) | (mg/dog) | (mg/dog) | (hr) | statin | Lactone | statin | Lactone |
| 1 | Atorvastatin | 4 | NA | 20 | 0.5 | 10.0 | 1.6 | 20.5 | 6.1 |
| | Calcium | | | | | | | | |
| | (Example 3) | | | | | | | | |
| 2 | Gemcabene | | 150 | NA | NA | NA | NA | NA | NA |
| | Calcium | | | | | | | | |
| | (Example 4) | | | | | | | | |
| 3 | PR 1 (Example 5) | 4 | 150 | 20 | 2 | 3.6 | 1.5 | 3.6 | 0.8 |
| 4 | PR 2 (Example 6) | 4 | 150 | 20 | 4 | 3.3 | 0.0 | 3.4 | 1.6 |
| 5 | LIPITOR ® | 4 | NA | 40 | 0.5 | 15.0 | 4.5 | 18.0 | 12.0 |
| | (Atorvastatin | | | | | | | | |
| | Calcium) | | | | | | | | |
| 6 | PR 3 (Example 7) | 4 | NA | 40 | 1 | 8.9 | 1.5 | 8.1 | 2.6 |
| 7 | PR 4 (Example 8) | 3 | NA | 40 | 1 | 17.6 | 2.8 | 21.2 | 7.5 |
| 8 | PR 5 (Example 9) | 4 | NA | 40 | 1 | 8.7 | 0.0 | 6.3 | 1.8 |

| | AUC₀₋ₜ (ng · hr/mL) | | | | | | | |
|---|---|---|---|---|---|---|---|---|
| | | 4-Hydroxy | AUC₀₋ₜ (ng · hr/mL) | | | Percent | | |
| | 4-Hydroxy | Atorva- | Total | Total | | Total | Total | |
| | Atorva- | statin | Atorva- | Non- | Total | Atorva- | Non- | Total |
| Phase | statin | Lactone | statin | Lactone | Lactone | statin | Lactone | Lactone |
| 1 | 1.8 | 0.9 | 40.8 | 32.3 | 8.5 | 100.0 | 79.2 | 20.8 |
| 2 | NA | NA | NA | NA | NA | NA | NA | NA |
| 3 | 0.0 | 0.0 | 9.4 | 7.2 | 2.2 | 100.0 | 76.5 | 23.5 |
| 4 | 0.0 | 0.0 | 8.3 | 6.7 | 1.6 | 100.0 | 80.3 | 19.7 |
| 5 | 16.0 | 8.5 | 74.0 | 49.0 | 25.0 | 100.0 | 66.2 | 33.8 |
| 6 | 1.4 | 1.1 | 23.6 | 18.4 | 5.2 | 100.0 | 78.0 | 22.0 |
| 7 | 1.8 | 2.6 | 53.5 | 40.6 | 12.9 | 100.0 | 75.8 | 24.2 |
| 8 | 1.0 | 0.0 | 17.8 | 16.0 | 1.8 | 100.0 | 90.1 | 9.9 |

N/A—indicates measurement is not applicable to this group.
0.0—indicates below the level of quantitation

TABLE 23

| | Composition | | Composition | | Composition | | Composition | |
|---|---|---|---|---|---|---|---|---|
| | Formula 1 | | Formula 2 | | Formula 3³ | | Formula 4² | |
| | (Released at pH 5.5) | | (Released at pH 6.0) | | (Released at pH 6.5) | | (Released at pH 7.0) | |
| | | mg/Unit | | mg/Unit | | mg/Unit | | mg/Unit |
| Ingredients | % w/w | Dose | % w/w | Dose | % w/w | Dose | % w/w | Dose |

Tablet Core Composition

| | | | | | | | | |
|---|---|---|---|---|---|---|---|---|
| Atorvastatin Calcium (Trihydrate)¹ | 12.10 | 21.87 | 12.10 | 21.87 | 12.10 | 21.87 | 12.10 | 21.87 |
| Calcium Carbonate | 19.91 | 36.0 | 19.91 | 36.0 | 19.91 | 36.0 | 19.91 | 36.0 |
| Lactose Monohydrate (Tabletose 70) | 35.47 | 64.13 | 35.47 | 64.13 | 35.47 | 64.13 | 35.47 | 64.13 |
| Sodium Starch Glycolate, Type A | — | — | — | — | — | — | — | — |
| Microcrystalline Cellulose (PH102) | 16.59 | 30.0 | 16.59 | 30.0 | 16.59 | 30.0 | 16.59 | 30.0 |
| Polyvinylpyrrolidone (Plasdone K29/32) | 1.66 | 3.0 | 1.66 | 3.0 | 1.66 | 3.0 | 1.66 | 3.0 |
| Polysorbate 80 | 0.22 | 0.4 | 0.22 | 0.4 | 0.22 | 0.4 | 0.22 | 0.4 |
| Croscarmellose Sodium | 2.21 | 4.0 | 2.21 | 4.0 | 2.21 | 4.0 | 2.21 | 4.0 |
| Magnesium Stearate, NF | 0.33 | 0.6 | 0.33 | 0.6 | 0.33 | 0.6 | 0.33 | 0.6 |

Sub-Coat (Barier) Composition

| | | | | | | | | |
|---|---|---|---|---|---|---|---|---|
| Opadry Clear (O3K19229) | 2.65 | 4.8 | 2.65 | 4.8 | 2.65 | 4.8 | 2.65 | 4.8 |

Enteric Coating Composition

| | | | | | | | | |
|---|---|---|---|---|---|---|---|---|
| Methacrylic Acid, Methyl Acrylate, Methyl | — | — | 1.88 | 3.4 | 2.45 | 4.422 | 3.77 | 6.82 |
| Methacrylate Polymer (Eudragit ® FS30D) | | | | | | | | |
| Methacrylic Acid Copolymer Type C | 5.53 | 10.0 | 3.76 | 6.8 | 3.20 | 5.782 | 1.89 | 3.42 |
| (Eudragit ® L30D55) | | | | | | | | |
| PlasACRYL ® HTP20 | — | — | — | — | — | — | — | — |
| Triethyl Citrate, NF | 0.55 | 1.0 | 0.39 | 0.7 | 0.39 | 0.7 | 0.39 | 0.7 |
| Talc USP/EP (Luzenac Pharma M) | 2.77 | 5.0 | 2.82 | 5.1 | 2.82 | 5.1 | 2.82 | 5.1 |
| Totals | 100.0 | 180.8 | 100.0 | 180.8 | 100.0 | 180.8 | 100.0 | 180.8 |

¹Atorvastatin Calcium Trihydrate dosed as Atorvastatin based on 99.6% assay (anhydrous basis) and 4.9% water content: 21.87 mg Atorvastatin Calcium Trihydrate = 20 mg Atorvastatin
²Formula 4 was prepared as Batch 121-16005 (Formulation 1) for the Dog PK study and used in the PR 1 test article.
³Formula 3 was prepared as Batch 121-16007 (Formulation 2) for the Dog PK study and used in the PR 2 test article.
⁴Formula 6 was prepared as Batch 121-17002 (Formulation 3) for the Dog PK study and used in the PR 3 test article.
⁵Formula 7 was prepared as Batch 121-17007 (Formulation 4) for the Dog PK study and used in the PR 4 test article.
⁶Formula 8 was prepared as Batch 121-17005 (Formulation 5) for the Dog PK study and used in the PR 5 test article.

TABLE 24

| | Composition | | Composition | | Composition | | Composition | |
|---|---|---|---|---|---|---|---|---|
| | Formula 5 | | Formula 6⁴ | | Formula 7⁵ | | Formula 8⁶ | |
| | (Released at pH 7.2) | | (Released at pH 7.0) | | (Released at pH 7.0) | | (Released at pH 7.0) | |
| | | mg/Unit | | mg/Unit | | mg/Unit | | mg/Unit |
| Ingredients | % w/w | Dose | % w/w | Dose | % w/w | Dose | % w/w | Dose |

Tablet Core Composition

| | | | | | | | | |
|---|---|---|---|---|---|---|---|---|
| Atorvastatin Calcium (Trihydrate)1 | 12.10 | 21.87 | 12.77 | 21.87 | 12.77 | 21.87 | 12.9 | 21.87 |
| Calcium Carbonate | 19.91 | 36.0 | 21.03 | 36.0 | 21.03 | 36.0 | 21.23 | 36.0 |
| Lactose Monohydrate (Tabletose 70) | 35.47 | 64.13 | 37.46 | 64.13 | 33.72 | 57.73 | 37.81 | 64.13 |
| Sodium Starch Glycolate, Type A | — | — | — | — | 3.74 | 6.4 | — | — |
| Microcrystalline Cellulose (PH102) | 16.59 | 30.0 | 17.52 | 30.0 | 17.52 | 30.0 | 17.69 | 30.0 |
| Polyvinylpyrrolidone (Plasdone K29/32) | 1.66 | 3.0 | 1.75 | 3.0 | 1.75 | 3.0 | 1.77 | 3.0 |
| Polysorbate 80 | 0.22 | 0.4 | 0.23 | 0.4 | 0.23 | 0.4 | 0.24 | 0.4 |
| Croscarmellose Sodium | 2.21 | 4.0 | 2.34 | 4.0 | 2.34 | 4 | 2.36 | 4.0 |
| Magnesium Stearate, NF | 0.33 | 0.6 | 0.35 | 0.6 | 0.35 | 0.6 | 0.35 | 0.6 |

Sub-Coat (Barier) Composition

| | | | | | | | | |
|---|---|---|---|---|---|---|---|---|
| Opadry Clear (O3K19229) | 2.65 | 4.8 | 2.8 | 4.8 | 2.8 | 4.8 | 2.83 | 4.8 |

Enteric Coating Composition

| | | | | | | | | |
|---|---|---|---|---|---|---|---|---|
| Methacrylic Acid, Methyl Acrylate, Methyl | 5.71 | 10.33 | 1.59 | 2.72 | 1.59 | 2.72 | 1.72 | 2.91 |
| Methacrylate Polymer (Eudragit ® FS30D) | | | | | | | | |
| Methacrylic Acid Copolymer Type C | — | — | 0.79 | 1.36 | 0.79 | 1.36 | 0.86 | 1.452 |
| (Eudragit ® L30D55) | | | | | | | | |
| PlasACRYL ® HTP20 | — | — | — | — | — | — | 0.26 | 0.438 |
| Triethyl Citrate, NF | 0.29 | 0.52 | 0.16 | 0.28 | 0.16 | 0.28 | — | — |
| Talc USP/EP (Luzenac Pharma M) | 2.85 | 5.15 | 1.19 | 2.04 | 1.19 | 2.04 | — | — |
| Totals | 100.0 | 180.8 | 100.0 | 171.2 | 100.0 | 171.2 | 100.0 | 169.6 |

1Atorvastatin Calcium Trihydrate dosed as Atorvastatin based on 99.6% assay (anhydrous basis) and 4.9% water content: 21.87 mg Atorvastatin Calcium Trihydrate = 20 mg Atorvastatin
²Formula 4 was prepared as Batch 121-16005 (Formulation 1) for the Dog PK study and used in the PR 1 test article.
³Formula 3 was prepared as Batch 121-16007 (Formulation 2) for the Dog PK study and used in the PR 2 test article.
⁴Formula 6 was prepared as Batch 121-17002 (Formulation 3) for the Dog PK study and used in the PR 3 test article.
⁵Formula 7 was prepared as Batch 121-17007 (Formulation 4) for the Dog PK study and used in the PR 4 test article.
⁶Formula 8 was prepared as Batch 121-17005 (Formulation 5) for the Dog PK study and used in the PR 5 test article.

1. A tablet comprising:
a core, the core comprising a statin or a pharmaceutically acceptable salt thereof; and
an outer coating, the outer coating comprising a first copolymer and a second copolymer,
the first copolymer comprising methyl acrylate, methyl methacrylate and methacrylic acid repeat units in a ratio of (about 7):(about 3):(about 1), and
the second copolymer comprising methacrylic acid and ethyl acrylate repeat units in a ratio of (about 1):(about 1),
wherein the core has an outer surface and wherein the outer coating is disposed over the entire outer surface; and
wherein the total amount of the first copolymer and the second copolymer ranges from about 2% to about 3% w/w of the tablet.

2.-5. (canceled)

6. The tablet of claim 1, wherein the total amount of the first copolymer and the second copolymer ranges from about 2.2% to about 2.8% w/w of the tablet.

7.-9. (canceled)

10. The tablet of claim 1, wherein the ratio of the first copolymer to the second copolymer is about 2:1 by weight.

11. The tablet of claim 1, wherein the amount of the first copolymer ranges from about 40 wt % to about 95 wt % of the outer coating.

12. The tablet of claim 1, wherein the amount of the second copolymer ranges from about 50 wt % to about 95 wt % of the outer coating.

13. The tablet of claim 1, wherein the total amount of the first copolymer and the second copolymer ranges from about 60 wt % to about 99.9 wt % of the outer coating.

14. The tablet of claim 1, wherein the first copolymer has a weight average molar mass of about 280,000 g/mol.

15. The tablet of claim 1, wherein the second copolymer has weight average molar mass of about 320,000 g/mol.

16. The tablet of claim 1, further comprising a subcoating, wherein the subcoating is disposed over the entire outer surface and the outer coating is disposed over the entire subcoating, the subcoating comprising hydroxypropyl methylcellulose (HPMC), hydroxypropylcellulose, polyvinyl alcohol, povidone, copovidone, methylcellulose, hydroxyethyl cellulose, starch, modified starches, sodium carboxymethylcellulose, guar or a combination thereof.

17. The tablet of claim 1, wherein the statin is atorvastatin, simvastatin, pravastatin, rosuvastatin, fluvastatin, lovastatin, dalvastatin, dihydrocompactin, cerivastatin or pitavastatin.

18. The tablet of claim 1, wherein the statin is atorvastatin.

19. The tablet of claim 1, wherein the pharmaceutically acceptable salt of the statin is atorvastatin calcium.

20. The tablet of claim 1, wherein the amount of the statin or pharmaceutically acceptable salt thereof ranges from about 5% to about 95% w/w of the core.

21. The tablet of claim 1, wherein the amount of the statin or pharmaceutically acceptable salt thereof ranges from about 10% to about 50% w/w of the core.

22. The tablet of claim 1, wherein the amount of the statin or pharmaceutically acceptable salt thereof ranges from about 10% to about 15% w/w of the core.

23. The tablet of claim 1, wherein the outer coating does not comprise a statin or a pharmaceutically acceptable salt thereof.

24. The tablet of claim 16, wherein the subcoating does not comprise a statin or a pharmaceutically acceptable salt thereof.

25. The tablet of claim 1, wherein when the tablet is subjected to dissolution testing according to USP <711> Delayed Release Dosage Forms Method A using Apparatus 2 (Paddle Apparatus) at 100 RPM, no more than 5% of the statin is detected in an acidic dissolution medium 2 hours after operation of Apparatus 2, wherein the acidic dissolution medium is 0.1N HCl.

26. The tablet of claim 25, wherein no more than 2% of the statin is detected in the acidic dissolution medium 2 hours after operation of the Apparatus 2.

27. The tablet of claim 25, wherein 0% of the statin is detected in the acidic dissolution medium 2 hours after operation of Apparatus 2.

28.-30. (canceled)

31. The tablet of claim 1, wherein the core comprises about 0.1 mg to about 80 mg of the statin or a pharmaceutically acceptable salt thereof.

32. The tablet of claim 1, wherein the tablet is a microtablet having a diameter ranging from about 1 mm to about 5 mm.

33. The tablet of claim 1, further comprising another pharmaceutically active agent.

34. The tablet of claim 33, wherein the outer coating does not comprise the other pharmaceutically acceptable active agent.

35. The tablet of claim 33, wherein the subcoating does not comprise the other pharmaceutically acceptable active agent.

36. The tablet of claim 33, wherein the other pharmaceutically active agent is ezetimibe, gemcabene or a pharmaceutically acceptable salt thereof.

37. The tablet of claim 33, wherein the other pharmaceutically active agent is gemcabene calcium.

38. The tablet of claim 37, further comprising ezetimibe or a pharmaceutically acceptable salt thereof.

39. The tablet of claim 33, wherein the core is a first core and the tablet further comprises a second core, wherein the second core comprises the other pharmaceutically active agent.

40. The tablet of claim 39, wherein the second core is not coated with the outer coating.

41. An oral dosage form comprising the tablet of claim 1 and a composition comprising another pharmaceutically active agent.

42. The oral dosage form of claim 41, wherein the other pharmaceutically active agent is ezetimibe, gemcabene or a pharmaceutically acceptable salt thereof.

43. The oral dosage form of claim 41, wherein the other pharmaceutically active agent is gemcabene calcium.

44. The oral dosage form of claim 43, further comprising ezetimibe or a pharmaceutically acceptable salt thereof.

45. The oral dosage form of claim 41, wherein:
the tablet is a first tablet,
the oral dosage form comprises a second tablet and
the second tablet comprises the other pharmaceutically active agent.

46. The oral dosage form of claim 41, further comprising a separation layer between the tablet and the composition, wherein the separation layer comprises hydroxypropyl methylcellulose (HPMC), hydroxypropylcellulose, polyvinyl alcohol, povidone, copovidone, methylcellulose, hydroxyethyl cellulose, starch, modified starch, sodium carboxymethylcellulose, guar or a combination thereof.

47. The tablet of claim 1, wherein the tablet when administered to a mammal provides, at a time point after the tablet's administration, a lower plasma concentration of total statin lactones than the mammal's plasma concentration of total statin lactones at the time point after administration of a tablet comprising a statin or a pharmaceutically acceptable salt thereof, but not comprising the outer coating.

48. The tablet of claim 18, wherein the tablet when administered to a mammal provides, at a time point after the tablet's administration, a lower plasma concentration of atorvastatin lactone, 2-hydroxyatorvastatin lactone or 4-hydroxyatorvastatin lactone than the subject's plasma concentration of the atorvastatin lactone, the 2-hydroxyatorvastatin lactone or the 4-hydroxyatorvastatin lactone at the time point after administration of a tablet comprising atorvastatin or a pharmaceutically acceptable salt thereof, but not comprising the outer coating.

49.-50. (canceled)

51. A capsule containing a tablet of claim 1.

52. The capsule of claim 51, further containing another pharmaceutically active agent.

53. The capsule of claim 52, wherein the pharmaceutically active agent is ezetimibe, gemcabene, or a pharmaceutically acceptable salt thereof.

54. The capsule of claim 52, wherein the other pharmaceutically active agent is gemcabene or a pharmaceutically acceptable salt thereof.

55. The capsule of claim 52, wherein the other pharmaceutically active agent is gemcabene calcium.

56. The capsule of claim 54, wherein the gemcabene or pharmaceutically acceptable salt thereof is present in an amount ranging from about 50 mg to about 900 mg per capsule.

57. The capsule of claim 54, further containing ezetimibe or a pharmaceutically acceptable salt thereof.

58.-61. (canceled)
